SEQUENCE LISTING (77 sequences)

SEQ ID NO: 1 - protein, 799 aa, Mus sp.
  MISC_FEATURE: FGFR4
MWLLLALLSIFQGTPALSLEASEEMEQEPCLAPILEQQEQVLTVALGQPVRLCCGRTERGRHWYKEGSRLASAGRVRGWRGRLEIASFLPEDAGRYLCLARGSMTVVHNLTLLMDDSLTSISNDEDPKTLSSSSSGHVYPQQAPYWTHPQRMEKKLHAVPAGNTVKFRCPAAGNPMPTIHWLKDGQAFHGENRIGGIRLRHQHWSLVMESVVPSDRGTYTCLVENSLGSIRYSYLLDVLERSPHRPILQAGLPANTTAVVGSDVELLCKVYSDAQPHIQWLKHVVINGSSFGADGFPYVQVLKTTDINSSEVEVLYLRNVSAEDAGEYTCLAGNSIGLSYQSAWLTVLPEEDLTWTTATPEARYTDIILYVSGSLVLLVLLLLAGVYHRQVIRGHYSRQPVTIQKLSRFPLARQFSLESRSSGKSSLSLVRGVRLSSSGPPLLTGLVNLDLPLDPLWEFPRDRLVLGKPLGEGCFGQVVRAEAFGMDPSRPDQTSTVAVKMLKDNASDKDLADLVSEMEVMKLIGRHKNIINLLGVCTQEGPLYVIVECAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAAPSQRPTFKQLVEALDKVLLAVSEEYLDLRLTFGPFSPSNGDASSTCSSSDSVFSHDPLPLEPSPFPFSDSQTT

SEQ ID NO: 2 - protein, 802 aa, Homo sapiens
  MISC_FEATURE: FGFR4 human isoform 1
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 3 - protein, 802 aa, Homo sapiens; identical to SEQ ID NO: 2
  MISC_FEATURE: FGFR4 human isoform 2

SEQ ID NO: 4 - protein, 800 aa, Rattus norvegicus
  MISC_FEATURE: FGFR4
MWLLLALLSIFQETPAFSLEASEEMEQEPCPAPISEQQEQVLTVALGQPVRLCCGRTERGRHWYKEGSRLASAGRVRGWRGRLEIASFLPEDAGRYLCLARGSMTVVHNLTLIMDDSLPSINNEDPKTLSSSSSGHSYLQQAPYWTHPQRMEKKLHAVPAGNTVKFRCPAAGNPMPTIHWLKNGQAFHGENRIGGIRLRHQHWSLVMESVVPSDRGTYTCLVENSLGSIRYSYLLDVLERSPHRPILQAGLPANTTAVVGSNVELLCKVYSDAQPHIQWLKHIVINGSSFGADGFPYVQVLKTTDINSSEVEVLYLRNVSAEDAGEYTCLAGNSIGLSYQSAWLTVLPAEEEDLAWTTATSEARYTDIILYVSGSLALVLLLLLAGVYHRQAIHGHHSRQPVTVQKLSRFPLARQFSLESRSSGKSSLSLVRGVRLSSSGPPLLTGLVSLDLPLDPLWEFPRDRLVLGKPLGEGCFGQVVRAEALGMDSSRPDQTSTVAVKMLKDNASDKDLADLISEMEMMKLIGRHKNIINLLGVCTQEGPLYVIVEYAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAAPSQRPTFKQLVEALDKVLLAVSEEYLDLRLTFGPYSPNNGDASSTCSSSDSVFSHDPLPLEPSPFPFPEAQTT

SEQ ID NO: 5 - protein, 799 aa, Mus sp.
  MISC_FEATURE: FGFR4 G to R substitution
MWLLLALLSIFQGTPALSLEASEEMEQEPCLAPILEQQEQVLTVALGQPVRLCCGRTERGRHWYKEGSRLASAGRVRGWRGRLEIASFLPEDAGRYLCLARGSMTVVHNLTLLMDDSLTSISNDEDPKTLSSSSSGHVYPQQAPYWTHPQRMEKKLHAVPAGNTVKFRCPAAGNPMPTIHWLKDGQAFHGENRIGGIRLRHQHWSLVMESVVPSDRGTYTCLVENSLGSIRYSYLLDVLERSPHRPILQAGLPANTTAVVGSDVELLCKVYSDAQPHIQWLKHVVINGSSFGADGFPYVQVLKTTDINSSEVEVLYLRNVSAEDAGEYTCLAGNSIGLSYQSAWLTVLPEEDLTWTTATPEARYTDIILYVSGSLVLLVLLLLARVYHRQVIRGHYSRQPVTIQKLSRFPLARQFSLESRSSGKSSLSLVRGVRLSSSGPPLLTGLVNLDLPLDPLWEFPRDRLVLGKPLGEGCFGQVVRAEAFGMDPSRPDQTSTVAVKMLKDNASDKDLADLVSEMEVMKLIGRHKNIINLLGVCTQEGPLYVIVECAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAAPSQRPTFKQLVEALDKVLLAVSEEYLDLRLTFGPFSPSNGDASSTCSSSDSVFSHDPLPLEPSPFPFSDSQTT

SEQ ID NO: 6 - protein, 800 aa, Rattus norvegicus
  MISC_FEATURE: FGFR4 G to R substitution
MWLLLALLSIFQETPAFSLEASEEMEQEPCPAPISEQQEQVLTVALGQPVRLCCGRTERGRHWYKEGSRLASAGRVRGWRGRLEIASFLPEDAGRYLCLARGSMTVVHNLTLIMDDSLPSINNEDPKTLSSSSSGHSYLQQAPYWTHPQRMEKKLHAVPAGNTVKFRCPAAGNPMPTIHWLKNGQAFHGENRIGGIRLRHQHWSLVMESVVPSDRGTYTCLVENSLGSIRYSYLLDVLERSPHRPILQAGLPANTTAVVGSNVELLCKVYSDAQPHIQWLKHIVINGSSFGADGFPYVQVLKTTDINSSEVEVLYLRNVSAEDAGEYTCLAGNSIGLSYQSAWLTVLPAEEEDLAWTTATSEARYTDIILYVSGSLALVLLLLLARVYHRQAIHGHHSRQPVTVQKLSRFPLARQFSLESRSSGKSSLSLVRGVRLSSSGPPLLTGLVSLDLPLDPLWEFPRDRLVLGKPLGEGCFGQVVRAEALGMDSSRPDQTSTVAVKMLKDNASDKDLADLISEMEMMKLIGRHKNIINLLGVCTQEGPLYVIVEYAAKGNLREFLRARRPPGPDLSPDGPRSSEGPLSFPALVSCAYQVARGMQYLESRKCIHRDLAARNVLVTEDDVMKIADFGLARGVHHIDYYKKTSNGRLPVKWMAPEALFDRVYTHQSDVWSFGILLWEIFTLGGSPYPGIPVEELFSLLREGHRMERPPNCPSELYGLMRECWHAAPSQRPTFKQLVEALDKVLLAVSEEYLDLRLTFGPYSPNNGDASSTCSSSDSVFSHDPLPLEPSPFPFPEAQTT

SEQ ID NO: 7 - DNA, 30 bp, Artificial Sequence
  neoR-specific primer
aggatctcctgtcatctcaccttcctcctg

SEQ ID NO: 8 - DNA, 30 bp, Artificial Sequence
  neoR-specific primer
aagaactcgtcaagaaggcgatagaaggcg

SEQ ID NO: 9 - DNA, 18 bp, Artificial Sequence
  Cre-specific primer
aacatgcttcatcgtcgg

SEQ ID NO: 10 - DNA, 20 bp, Artificial Sequence
  Cre-specific primer
ttaggatcatcagctacacc

SEQ ID NO: 11 - DNA, 21 bp, Artificial Sequence
  FGFR4-primer forward
cgtggacaacagcaacccctg

SEQ ID NO: 12 - DNA, 22 bp, Artificial Sequence
  FGFR4-primer reverse
gctggcgagagtagtggccacg

SEQ ID NO: 13 - DNA, 21 bp, Artificial Sequence
  TGF-alpha primer forward
tgtcaggctctggagaacagc

SEQ ID NO: 14 - DNA, 21 bp, Artificial Sequence
  TGF-alpha primer reverse
cacagcgaacacccacgtacc

SEQ ID NO: 15 - DNA, 17 bp, Artificial Sequence
  PymT primer forward
tcgccgcctaagactgc

SEQ ID NO: 16 - DNA, 18 bp, Artificial Sequence
  PymT primer reverse
ccgccctgggaatgatag

SEQ ID NO: 17 - DNA, 20 bp, Artificial Sequence
  HPRT primer fw
ataagccagactttgttgga

SEQ ID NO: 18 - DNA, 22 bp, Artificial Sequence
  HPRT primer rev
ccacttgaactctcatcttagg

SEQ ID NO: 19 - DNA, 21 bp, Artificial Sequence
  GAPDH primer fw
ccaatatgattccacccatgg

SEQ ID NO: 20 - DNA, 21 bp, Artificial Sequence
  GAPDH primer rev
ccttctccatggtggtgaaga

SEQ ID NO: 21 - DNA, 23 bp, Artificial Sequence
  FGFR4 primer for Light Cycler(R) fw
gcttatggatgactccttaccct

SEQ ID NO: 22 - DNA, 21 bp, Artificial Sequence
  FGFR4 primer for Light Cyler (R) rev
aatgcctccaatacgattctc

SEQ ID NO: 23 - DNA, 18 bp, Artificial Sequence
  E-Cadherin primer fw
gctggaccgagagagtta

SEQ ID NO: 24 - DNA, 19 bp, Artificial Sequence
  E-Cadherin primer rev
tcgttctccactctcacat

SEQ ID NO: 25 - DNA, 22 bp, Artificial Sequence
  N-Cadherin primer fw
ccacagacatggaaggcaatcc

SEQ ID NO: 26 - DNA, 23 bp, Artificial Sequence
  N-Cadherin primer rev
cactgattctgtatgccgcattc

SEQ ID NO: 27 - DNA, 19 bp, Artificial Sequence
  MMP14 primer fw
cgttcgctgctggacaagg

SEQ ID NO: 28 - DNA, 21 bp, Artificial Sequence
  MMP14 primer rev
gactgagaagggaggctggag

SEQ ID NO: 29 - DNA, 21 bp, Artificial Sequence
  MMP13 primer fw
tccctggaattggcaacaaag

SEQ ID NO: 30 - DNA, 24 bp, Artificial Sequence
  MMP13 primer rev
ggaatttgttggcatgactctcac

SEQ ID NO: 31 - DNA, 19 bp, Artificial Sequence
  MMP9 primer fw
ccctggaactcacacgaca

SEQ ID NO: 32 - DNA, 20 bp, Artificial Sequence
  MMP9 primer rev
ggaaactcacacgccagaag

SEQ ID NO: 33 - DNA, 24 bp, Artificial Sequence
  CD44 primer fw
ttgaatgtaacctgccgctacgca

SEQ ID NO: 34 - DNA, 23 bp, Artificial Sequence
  CD44 primer rev
tcggatccatgagtcacagtgcg

SEQ ID NO: 35 - DNA, 20 bp, Artificial Sequence
  flk-1 primer fw
tcgtgcgtgacatcaaagag

SEQ ID NO: 36 - DNA, 20 bp, Artificial Sequence
  flk-1 primer rev
tggacagtgaggccaggatg

SEQ ID NO: 37 - DNA, 21 bp, Artificial Sequence
  CDK4 primer fw
tggctgccactcgatatgaac

SEQ ID NO: 38 - DNA, 21 bp, Artificial Sequence
  CDK4 primer rev
cctcaggtcctggtctatatg

SEQ ID NO: 39 - DNA, 20 bp, Artificial Sequence
  Rb primer fw
catctaatggacttccagag

SEQ ID NO: 40 - DNA, 20 bp, Artificial Sequence
  Rb primer rev
cataacagtcctaactggag

SEQ ID NO: 41 - DNA, 21 bp, Artificial Sequence
  p21 primer fw
cgttttcggccctgagatgtt

SEQ ID NO: 42 - DNA, 22 bp, Artificial Sequence
  p21 primer rev
acccgggtccttcttgtgtttc

SEQ ID NO: 43 - DNA, 24 bp, Artificial Sequence
  p53 primer fw
aaccgccgacctatccttaccatc

SEQ ID NO: 44 - DNA, 24 bp, Artificial Sequence
  p53 primer rev
aggccccactttcttgaccattgt

SEQ ID NO: 45 - DNA, 21 bp, Artificial Sequence
  cyclin D1 primer fw
tcccgctggccatgaactacc

SEQ ID NO: 46 - DNA, 21 bp, Artificial Sequence
  cyclin D1 primer rev
ggcgcaggcttgactccagaa

SEQ ID NO: 47 - DNA, 18 bp, Artificial Sequence
  CDK1 primer fw
ccatgaactgcccaggag

SEQ ID NO: 48 - DNA, 22 bp, Artificial Sequence
  CDK1 primer rev
cggtgtggtgtataagggtaga

SEQ ID NO: 49 - DNA, 20 bp, Artificial Sequence
  CDK2 primer fw
cgataacaagctccgtccat

SEQ ID NO: 50 - DNA, 20 bp, Artificial Sequence
  CDK2 primer rev
agaagtggctgcatcacaag

SEQ ID NO: 51 - protein, 21 aa, Artificial Sequence
  blocking peptide
MRLLLALLGVLLSVPGPPVLS

SEQ ID NO: 52 - protein, 21 aa, Artificial Sequence
  blocking peptide
LEASEEVELEPCLAPSLEQQE

SEQ ID NO: 53 - protein, 12 aa, Artificial Sequence
  blocking peptide
QELTVALGQPVR

SEQ ID NO: 54 - protein, 14 aa, Artificial Sequence
  blocking peptide
LCCGRAERGGHWYK

SEQ ID NO: 55 - protein, 17 aa, Artificial Sequence
  blocking peptide
EGSRLAPAGRVRGWRGR

SEQ ID NO: 56 - protein, 19 aa, Artificial Sequence
  blocking peptide
LEIASFLPEDAGRYLCLAR

SEQ ID NO: 57 - protein, 19 aa, Artificial Sequence
  blocking peptide
GSMIVLQNLTLITGDSLTS

SEQ ID NO: 58 - protein, 16 aa, Artificial Sequence
  blocking peptide
SNDDEDPKSHRDPSNR

SEQ ID NO: 59 - protein, 15 aa, Artificial Sequence
  blocking peptide
HSYPQQAPYWTHPQR

SEQ ID NO: 60 - protein, 15 aa, Artificial Sequence
  blocking peptide
MEKKLHAVPAGNTVK

SEQ ID NO: 61 - protein, 14 aa, Artificial Sequence
  blocking peptide
FRCPAAGNPTPTIR

SEQ ID NO: 62 - protein, 13 aa, Artificial Sequence
  blocking peptide
WLKDGQAFHGENR

SEQ ID NO: 63 - protein, 7 aa, Artificial Sequence
  blocking peptide
IGGIRLR

SEQ ID NO: 64 - protein, 16 aa, Artificial Sequence
  blocking peptide
HQHWSLVMESVVPSDR

SEQ ID NO: 65 - protein, 15 aa, Artificial Sequence
  blocking peptide
GTYTCLVENAVGSIR

SEQ ID NO: 66 - protein, 14 aa, Artificial Sequence
  blocking peptide
YNYLLDVLERSPHR

SEQ ID NO: 67 - protein, 20 aa, Artificial Sequence
  blocking peptide
PILQAGLPANTTAVVGSDVE

SEQ ID NO: 68 - protein, 17 aa, Artificial Sequence
  blocking peptide
LLCKVYSDAQPHIQWLK

SEQ ID NO: 69 - protein, 21 aa, Artificial Sequence
  blocking peptide
HIVINGSSFGADGFPYVQVLK

SEQ ID NO: 70 - protein, 20 aa, Artificial Sequence
  blocking peptide
TADINSSEVEVLYLRNVSAE

SEQ ID NO: 71 - protein, 20 aa, Artificial Sequence
  blocking peptide
DAGEYTCLAGNSIGLSYQSA

SEQ ID NO: 72 - protein, 20 aa, Artificial Sequence
  blocking peptide
WLTVLPEEDPTWTAAAPEAR

SEQ ID NO: 73 - protein, 25 aa, Artificial Sequence
  blocking peptide
YTDIILYASGSLALAVLLLLAGLYR

SEQ ID NO: 74 - DNA, 3772 bp, Mus musculus
  mRNA (1)..(3772): transforming growth factor alpha (TGF alpha)
gcgcctttttcccccgcgcacaccgcggcggcgcgcggctactcgccaaccgcagggagcgcggtggctgcagcaccctgcgctcggaagatggtccccgcgaccggacagctcgctctgctagcgctgggtatcctgttagctgtgtgccaggctctggagaacagcacatcccccctgagtgactcacccgtggcggctgcagtggtgtctcacttcaacaagtgcccagattcccacactcagtactgcttccatggaacctgccggtttttggtgcaggaagagaagccagcatgtgtctgccactctgggtacgtgggtgttcgctgtgagcatgcagacctcctggctgtggtggctgccagccagaagaagcaagccatcactgccctggtggtggtctccattgtggccctggctgtcctcattatcacctgtgtgctgatccactgctgtcagctccgcaaacactgtgagtggtgccgtgccctcgtctgcagacatgagaagcccagcgccctcctgaagggaaggactgcttgctgccactctgagacagtggtctgaagatcccagaggaggaatttggccaggtggcctatgacagcccaaccaagaaaaggcatcttgggacaacacccctggcagtgcccaggcccatgggacatgctgggagaccttccccctcagtgcacaactgcctgggcaggcttcttccttgagagtcttcaaaactgtgtgataaagctgcctgctgggctcgctcagtacacccagagaagaggccagtggaccacattttaaagacaagttgaacacgaacctcaaagggttggccttcttgctaacccacaccgagaatgagctggggctgctgtcccctgccagccatgacttccagactgtttcttctctatgggccatctacccccccgtcccgagactccatggttggtgtacaaaatggacaaggggaaacgtctattgttctttgaagacaccatggcgtcccatgcgccctgacatctcctcaggctgtcgtcaggatgcgtgtcttatttattagatggataacatggttttatttgtaatctctttatgtcaatgtcaggcgtccatgctagcgatgcatgagtggcgggccgtgaagttacaccaaatagaaatgtccagccactttgataaagtaaggctgcagggaggggagcctccggggggccgtagagacagaattcagatgtgcatcctgctgggaaccaacctgctatcccaaggctgtgccccctcaggcaagaaagtccctggatacatgctgtagcaaacttagcaaggccaatggcagagagtgtcccccttgtggggacaagaggacaaaagagcctgtgtgggcacctaggaaggatgcacaccccaacccttgattaagtggattagcgtcctcgtgagcccagctccagcatcagcaatgagctgtgagccaaatccatgaaggggaccagccctttattggcttttgcttatttttagaaggagaaatttgcctttcctatttattttcaagcattaagaaaaatagcccactgttttgttttgtttcccctttaataatgttggcattattaacaaaatcgctaccaagcctctccacccctctctggggaaggaaatgtgttccctgagcacccgaagatgctctgacccattgagcccacccttgttcctgggctgcttgggactgtcaggagacagaatacattcagttgcatattaaagagaccattctccaactcagaagtgaaaagtctgccccctcggccctccagggcctctgaagtaaggcatgagggaaggggtactgggcataccaagttggtttgaagcttctgtcagaagctgcctgttcctcattcttctcctttatgaatttttctcctctcatatgatctaggcattgatctgcccaggtcttaaccctttgttcacaaggcctgaagccctgaaagaagaacctgaaactgttcagtgctgtcctctaactgcttagctgacacagctccaggcagaagttgtcattcttgctattattttggtttgatgctttatcttgttttgtgagacaaggtcttgctgtgtagcccaggttgacctttaagttaagatcctgcttcagcaacctggaagctgggcttacagatgtgcaccaccacgcccagcactttttgattactagagaaaataggttaagtttgctttccttgggtcatacaagtgggtcaaggccaagtgtcacctgctgctcagacaccaatcaacctcactggacttcagccctctattggcccccagtgctctcagtagaccccgcctgaagtactggctcactggagggcagggagcaacacaaatggaggagatgtggggcttgttttagtttagttcacactgttagcttctgatgacaggtcttttctggtctgctggaggctcctgggtgtctgacttcttttaaagagtttgaactgatatcaaaatatcattgggaacttcctcacttaatatcccatcctgaagatgttgtcatccaagactaagcagaggtggtaaacgcttagcacgagcactcgatggtgaaggcagaggatctcgggaggacctcagctttagtgtccaaacggtcctgttctcaggtccagccagtcgcagcagccagtaccatcccagtgcctgtacacagatgcagctggctcaaacacctcccagaagtgccttcccatcagtctaccctttgcagagtcagaggctgagactaggttgagacaagtccggttggacatcgtccctgccccgacgtatgacagccctgctgcgttggtgactttgattttggaagtcagtcagacaaacgctgtgtaccttctcctgtgccatgtcacagcacagatgggcctggttagctgtaccagcgccctcctgtcagttcacactggccctctcttgatatttagtacagctacttaatcaggagcactcttagcagctaggcaggtcagagggcagtacagttgattcagtgactcaggggactagggccattcccattaaatgagctgaaattaattctagagacattgagacagctcagttagcaatggcacttaccaaaatcccagataacctgaattcaatcccctgggatcccatctggtggaaggagagaaccggcttgcccaggatgtcttctgatttctacgtacacgcacacgcacacacacgcacgcgcacacacacgcacacacacgcacatatgagtacacacaaagtctaagtttttaaaataaaaattctagaaagacctctacacctctagctataactttaaacacaccatgggacatgttttctccacggaaaagcacaattggagacccagtctgaaatgaaagatagattccaggtggacatacacagaaccaaaccatgagttagtttgtcatgtgccccagaaaaaggtgggaacaataatcccaacatccctttcctctccccccccaaaaaagggggggaggaggcatggttggtacccacccacctgcactggtgagggaagtgactatgtccctgagtcctagatggtgacgttggtcccttcaagtccccaggccaggtttcctagtgcctatgaggcactggatagaccagcaacactctgacactgcctacaggccccagtcctgtaatgagtaataaacttgtctctctcccaaggcca

SEQ ID NO: 75 - protein, 159 aa, Mus musculus
  MISC_FEATURE (1)..(159): TGF alpha
MVPATGQLALLALGILLAVCQALENSTSPLSDSPVAAAVVSHFNKCPDSHTQYCFHGTCRFLVQEEKPACVCHSGYVGVRCEHADLLAVVAASQKKQAITALVVVSIVALAVLIITCVLIHCCQLRKHCEWCRALVCRHEKPSALLKGRTACCHSETVV

SEQ ID NO: 76 - DNA, 5983 bp, Mus musculus
  mRNA (1)..(5983): epidermal growth factor receptor (EGFR), transcript variant 1
ctcccccagtcccgacccgagctaactagacgtctgggcagccccagcgcaacgcgcagcagcctccctcctcttcttcccgcactgtgcgctcctcctgggctagggcgtctggatcgagtcccggaggctaccgcctcccagacagacgacaggtcacctggacgcgagcctgtgtccgggtctcgtcgttgccggcgcagtcactgggcacaaccgtgggactccgtctgtctcggattaatcccggagagccagagccaacctctcccggtcagagatgcgaccctcagggaccgcgagaaccacactgctggtgttgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcaggggaccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggccaaagataccatctattgccactgggattgtgggtggcctcctcttcatagtggtggtggcccttgggattggcctattcatgcgaagacgtcacattgttcgaaagcgtacactacgccgcctgcttcaagagagagagctcgtggaacctctcacacccagcggagaagctccaaaccaagcccacttgaggatattaaaggaaacagaattcaaaaagatcaaagttctgggttcgggagcatttggcacagtgtataagggtctctggatcccagaaggtgagaaagtaaaaatcccggtggccatcaaggagttaagagaagccacatctccaaaagccaacaaagaaatccttgacgaagcctatgtgatggctagtgtggacaaccctcatgtatgccgcctcctgggcatctgtctgacctccactgtccagctcattacacagctcatgccctacggttgcctcctggactacgtccgagaacacaaggacaacattggctcccagtacctcctcaactggtgtgtgcagattgcaaagggcatgaactacctggaagatcggcgtttggtgcaccgtgacttggcagccaggaatgtactggtgaagacaccacagcatgtcaagatcacagattttgggctggccaaactgcttggtgctgaagagaaagaatatcatgccgaggggggcaaagtgcctatcaagtggatggctttggaatcaattttacaccgaatttatacacaccaaagtgatgtctggagctatggtgtcactgtgtgggaactgatgacctttgggtccaagccttatgatggaatcccagcaagtgacatctcatccatcctagagaaaggagagcgccttccacagccacctatctgcaccatcgatgtctacatgatcatggtcaagtgctggatgatagatgctgatagccgcccaaagttccgagagttgattcttgaattctccaaaatggcccgagacccacagcgctaccttgttatccagggggatgaaagaatgcatttgccaagccctacagactccaacttttaccgagccctgatggatgaagaggacatggaggatgtagttgatgctgatgagtatcttatcccacagcaaggcttcttcaacagcccgtccacgtcgaggactcccctcttgagttctctgagtgcaactagcaacaattccactgtggcttgcattaatagaaatgggagctgccgtgtcaaagaagacgccttcttgcagcggtacagctccgaccccacaggtgctgtaacagaggacaacatagatgacgcattcctccctgtacctgaatatgtaaaccaatctgttcccaagaggccagcaggctctgtgcagaaccctgtctatcacaatcagcccctgcatccagctcctggaagagacctgcattatcaaaatccccacagcaatgcagtgggcaaccctgagtatctcaacactgcccagcctacctgtctcagtagtgggtttaacagccctgcactctggatccagaaaggcagtcaccaaatgagcctagacaaccctgactaccagcaggacttcttccccaaggaaaccaagccaaatggcatatttaagggccccacagctgaaaatgcagagtacctacgggtggcacctccaagcagtgagtttattggagcatgacaagaaggggcatcataccagctataaaatgtctggactttctagaatcccaggaccaactatggcagcacctccacttctggtagccatgcccacgctgtgtcaaatgtcactcagactggctttaaagcataactctgatgggctttgtcactgagccaagaagtgggcctctctcctgatgcactttgggaagttgaaggtacatcaattgatcttcgaactgtgaagattccacaaaaaaggtatccatcgagaacattgtccattggaacagaagtttgcctcatggtgaggtacatatgggaaaaaaacagacatatggagcttatatttagggaactttgggattcttgtctttattgatttgattgatgcactcttgtagtctggtacacagagttgcctggagccaactgaccagacagttggttccaccagctctgcatcaagacacttccgtggcaagacaactaaatgtataagaagtccatggatgccctgagcaggccacacttgtacagcattaaaccatggcagatacaataggataagccactttgttacttactggggctgggagaagaggaatgacggggtagaattttccctcagacgtactttttatataaatatgtccctggcacctaacacgcgctagtttaccagtgttttctattagacttccttctatgttttctgtttcattgttttgagttgtaaatatgtgttcctgtcttcatttcatgaagtaaacaaacaaacaaaaaacccagtattaagtattatcaaagaacaaccatgattccacattcgaacccattcaaaccatcagtattgtgaccaaaagcctttaactaagaaggagtaaccatgcaaaaatccatagaggaatttaacccaaaattttagtctcagcattgtgtctgctgaggtgtgtatatgagactacgaaagtgaactactcttcaaatccactttgccttcactcctctataccctaaatctagtgtaaaccacacatggaggataacttttttttttaattttaaaagtgtttattagatatgtttttcttcctggtaaactgcagccaaacatcagttaagagccatttttgataaacactatcacaatgatctcgggatccatcctttccgatttaccaagtgatggatagacgtgaactcataaacactacccataagacaaaacaatgagtgccagacaagacatcagccaggcaccagagcacagagcaggactgggcaatctgttggagatatctagaaagttcacaaaggaaacaagattgtccactaccttgtgagatctagcagtcataaataccagggaaatggaaagtgtgtttccttacagcaccaggtcttcgatcttcctaatgctgtgaccctttaatacagtttgccatgttgtggtgacccccaaccataaaattatttttgttgctacttcataactgtaaatttgctactcttacagaccacaatgtaaatatctgatatgctatctgatatgcaggctatctgacagaggtcgcaacccgcaggttgagagccactgccttcaaggctttaatcaagagagtagtgagctgagggctttactggtaagtcaggggcaagtccaactcaatcatcctcacatactggctgctccctcaggcctgagaatgaggcttgcagcatcctctggtttcctaaccgttatccatccctgactctcatctctgaaaatagatgtcatccatgaaattaaggagtgagaatattaagcagcatttatagagctcaaaattccatgtcatcaccaggaagtgccatgttgatcacagagaacacagaggagacatatagacagggttttgctcaaaattgggatatagaatgagcctgtcaggtacctatcaggagcggtaatccgtgagagagaaccgttgcaagccactctaactgtagcaatgaaaccctagtatttttgtactttgaaatactttcttataacaaaataaagtagcaaaaaaactgttcaaaaaaaaaaaaaaaaaaa

SEQ ID NO: 77 - protein, 1210 aa, Mus musculus
  MISC_FEATURE (1)..(1210): EGFR isoform 1
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGPKIPSIATGIVGGLLFIVVVALGIGLFMRRRHIVRKRTLRRLLQERELVEPLTPSGEAPNQAHLRILKETEFKKIKVLGSGAFGTVYKGLWIPEGEKVKIPVAIKELREATSPKANKEILDEAYVMASVDNPHVCRLLGICLTSTVQLITQLMPYGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASDISSILEKGERLPQPPICTIDVYMIMVKCWMIDADSRPKFRELILEFSKMARDPQRYLVIQGDERMHLPSPTDSNFYRALMDEEDMEDVVDADEYLIPQQGFFNSPSTSRTPLLSSLSATSNNSTVACINRNGSCRVKEDAFLQRYSSDPTGAVTEDNIDDAFLPVPEYVNQSVPKRPAGSVQNPVYHNQPLHPAPGRDLHYQNPHSNAVGNPEYLNTAQPTCLSSGFNSPALWIQKGSHQMSLDNPDYQQDFFPKETKPNGIFKGPTAENAEYLRVAPPSSEFIGA

(19) United States
(12) Patent Application Publication
(10) Pub. No.: US 2011/0179505 A1
(43) Pub. Date: Jul. 21, 2011

(54) RODENT CANCER MODEL FOR HUMAN FGFR4 ARG388 POLYMORPHISM

(75) Inventors: Axel Ullrich, Munich (DE); Thomas Mayer, Munich (DE); Sylvia Streit, Martinsried (DE); Nina Seitzer, Munich (DE)

(73) Assignee: MAX-PLANCK-GESELLSCHAGFT zur FOERDERUNG der WISSENSCHAFTEN e.V., Munich (DE)

(21) Appl. No.: 13/121,202

(22) PCT Filed: Sep. 29, 2009
(86) PCT No.: PCT/EP2009/062648

§ 371 Date: Mar. 28, 2011

(30) Foreign Application Priority Data
Sep. 29, 2008 (EP) 08017135.8

(51) Int. Cl. A01K 67/00; A01K 67/027; C12N 5/10; A61K 38/22; C07H 21/00; C12N 15/85; G01N 33/574; C07H 21/02; C07K 16/18

(52) U.S. Cl. 800/10; 800/14; 800/18; 435/352; 435/353; 435/354; 800/9; 530/399; 536/23.1; 435/320.1; 435/7.23; 536/24.5; 530/389.2

(57) ABSTRACT

The present invention provides a rodent animal for studying the molecular mechanisms and physiological processes associated with uncontrolled cell growth, e.g. cancer, and with a modified FGFR4.

### STATE OF THE ART

The fibroblast growth factor receptor (FGFR) signaling system is composed of four receptors (FGFR1-4) and more than 20 ligands and has been implicated in the regulation of various physiological processes including angiogenesis, mitogenesis, differentiation and development (1,2).

In human cancer, the FGFRs are implicated either by overexpression like, pancreatic- or prostate carcinoma (3-5), or by activating mutations leading to abnormal fusion proteins or nucleotide substitutions (6,7). In recent years, it has become clear that beside somatic mutations, germline mutations like single nucleotide polymorphisms (SNP) have an increasingly recognized significance for diseases like cancer but also in the determination of the response to therapeutic agents (8,10).

In the human FGFR4, a polymorphic nucleotide change in codon 388 substitutes Glycine (Gly) to Arginine (Arg) in the transmembrane region of the receptor, a hot spot in receptor tyrosine kinases (RTKs) for disease-relevant sequence variations (11). This single substitution in the FGFR4 was shown to be implicated in progression and poor prognosis of various types of human cancer. Here, Bange and colleagues could associate the FGFR4 Arg388 allele with tumor progression in breast and colon cancer patients (11). Similarly, soft tissue sarcoma patients, who carried the FGFR4 Arg388 allele had a poor clinical outcome (10). In melanoma, the Arg-allele is associated with increased tumor thickness, while in head and neck squamous cell carcinoma the Glycine-Arginine substitution results in reduced overall patient survival and advanced tumor stage. Furthermore, a recent study on prostate cancer patients strongly associated the FGFR4 Arg-allele not only with tumor progression but also with prostate cancer initiation. Breast cancer studies correlate the Arg-allele not only with accelerated disease progression but also with higher resistance to adjuvant systemic or chemotherapies in primary breast cancer leading to a significantly shorter disease-free and overall survival (11,16).

The main conclusion of these studies was that the presence of one or two Arg388 alleles in the genome of an individual does not initiate cancer development but predisposes the carrier to a more aggressive form if she or he is affected by the disease. Unfortunately, due to the highly complex and heterogeneous genetic background of the studies was at times marginal and because of difference in patient stratification and statistical evaluation, led to controversies (17,18).

The consequences of genetic modifications of the FGFR4 are described in humans suffering from different cancers (see above), however the molecular and biochemical mechanisms and the physiological processes behind them are not understood. Since an impact of the FGFR4 Arg388 allele on tumor progression is shown in correlative clinical studies, understanding the molecular and biochemical alterations underlying such FGFR4 modifications is fundamental for the prognosis on disease, the development of therapeutic strategies, and further cancer research.

Thus, there is a need for an animal as a model to study the molecular and biochemical effects of the FGFR4 modifications, particularly SNPs, leading to amino acid substitutions, in order to develop novel therapeutic strategies, to identify diagnostic markers and agents useful in disease treatment, and to gain more insight in the onset and progression of cancer but also of further diseases associated with FGFR4 modifications.

### SUMMARY OF THE INVENTION

In order to satisfy this need, the present invention provides a rodent animal comprising an endogenous gene encoding a modified FGFR4 protein, wherein the modification is an amino acid substitution in the wild-type FGFR4 of said rodent at the amino acid position corresponding to amino acid position 385 of SEQ ID NO: 1.

In one embodiment, the invention provides a rodent which is a mouse or a rat comprising an endogenous gene encoding a modified FGFR4 protein, wherein the modification is an amino acid substitution in the wild-type FGFR4 of said mouse or rat wherein in case of said mouse the amino acid substitution is at the amino acid position 385 of SEQ ID NO: 1 or wherein in case of said rat the amino acid substitution is at the amino acid position 386 of SEQ ID NO: 4.

In one embodiment, the invention provides a rodent which is a mouse comprising an endogenous gene encoding a modified FGFR4 protein, wherein the modification is an amino acid substitution in the wild-type FGFR4 of said mouse wherein the amino acid substitution is at the amino acid position 385 of SEQ ID NO: 1.

In an further aspect the invention relates to a rodent animal comprising an endogenous gene encoding a modified FGFR4 protein, wherein the modification is at least one amino acid substitution at any amino acid position compared to the wild-type FGFR4 protein, preferably an FGFR4 protein according to SEQ ID NO: 1, which modification, if present in at least some or all or essentially all cells of said animal in a heterozygous or homozygous manner, results in a phenotype associated with an alteration in tumor progression and/or formation, e.g. an increased rate of tumor growth and/or metastasis formation compared to a wild-type animal. In a preferred embodiment, said animal additionally expresses in the genome of at least some of its cells a transgene encoding a TGF-α protein.

The invention further relates to modified FGFR4 polypeptide and nucleic acid molecules encoding such modified FGFR4 proteins, as well as primary cells and cell lines derived from an non-human animal as described herein, e.g., a rodent.

The invention further pertains to the use of the animals, primary cells, or cell lines described herein as a model for:
(a) studying the molecular mechanisms of, or physiological processes associated with uncontrolled cell growth, such as cancer and/or metastasis formation, preferably in breast cancer, lung cancer, colorectal cancer, hepatocellular cancer, prostate cancer, melanoma, and/or pancreatic cancer;
(b) identification and/or testing of an agent useful in the prevention, amelioration or treatment of uncontrolled cell growth, such as cancer and/or metastasis formation, preferably in breast cancer, lung cancer, colorectal cancer, hepatocellular cancer, prostate cancer, melanoma, and/or pancreatic cancer;
(c) identification of a protein and/or nucleic diagnostic marker for uncontrolled cell growth, such as cancer and/or metastasis formation, preferably in breast cancer, lung cancer, colorectal cancer, hepatocellular cancer, prostate cancer, melanoma, and/or pancreatic cancer; and/or
(d) studying the molecular mechanisms of, or physiological processes or medical conditions associated with undesirable activity, expression, or production of said modified FGFR4.

### BRIEF DESCRIPTION OF THE FIGURES

FIG. 1: Generation of the FGFR4 Arg385 KI Mouse
(A) FGFR4 wt locus spanning exon 2 to 12 of murine FGFR4 genomic sequence; FGFR4 Arg385 KI gene-targeting construct, exon 8 contains the SNP established via specific mutagenesis, selection-cassette flanked by loxP-sites for Cre-deletion is introduced between exon 10 and 11; Neo: Neomycin-resistance; TK: thymidin-kinase-cassette
(B) 1) Southern Blot analysis of ES-cell clones after gene targeting; positive clones show a an additional 10 kb detected by 5′ external probe 2) genotyping of ES-cell clones via PCR-restriction fragment length polymorphism (RFLP), positive clones contain an additional fragment of 96 by
(C) Segregation analysis of FGFR4 Arg385 KI mice; FGFR4 Arg385 KI is inherited with a mendelian ratio in back (1:1)—and intercrosses (1:2:1)
(D) 1) Genotyping of FGFR4 Arg385 KI mice; Amplification product was cut by Mval restriction-enzyme to obtain specific banding to distinguish the FGFR4 alleles 2) Conformation of the TGFα transgen; 3) crossing scheme of FGFR4 Arg385 KI mice and oncomice transgenic for TGFα; Transgen was only inherited by males to ensure normal lactating of the females.

FIG. 2: Characterisation of the FGFR4 Arg385 KI Mouse
(A) mRNA expression levels in different tissues of adult FGFR4 Arg385 KI mice quantified by LightCycler® analysis; Expression levels are normalised on HPRT-housekeeping gene and plotted as absolute values; FGFR4 is expressed in various tissues; no differences detectable between the different FGFR4 alleles
(B) Protein-expression levels in different tissues of adult FGFR4 Arg385 KI mice analysed by immunoprecipitation and Western Blotting of FGFR4; Actin served as a loading control; no differences detectable between the different FGFR4 alleles
(C) Table of FGFR4 expression pattern in different tissues of adult FGFR4 Arg385 KI mice; FGFR4, immunohistochemically analysed, is expressed in various tissues; no differences detectable between the different FGFR4 alleles
(D) Lung and mammary gland tissue of adult FGFR4 Gly/Gly, Gly/Arg or Arg/Arg385 KI mice immunohistochemically analysed for FGFR4 expression; no differences detectable between the different FGFR4 alleles

FIG. 3: FGFR4 Arg385 enhances Mass and Size of occurring Tumors in the WAP-TGFα/EGFR Mouse Mammary Tumor Model

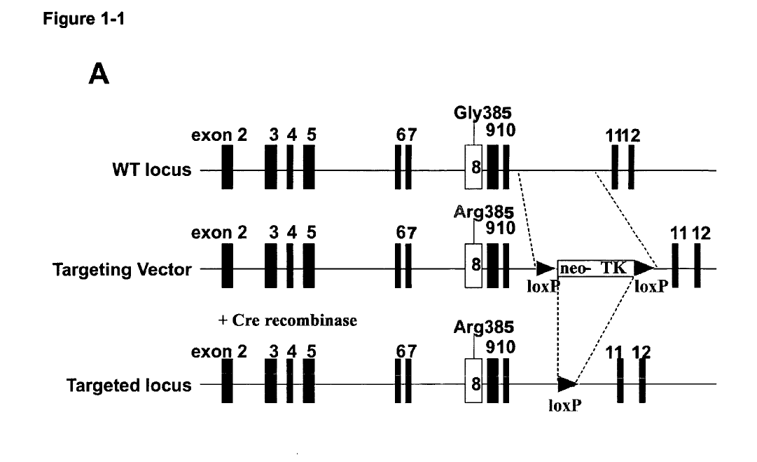

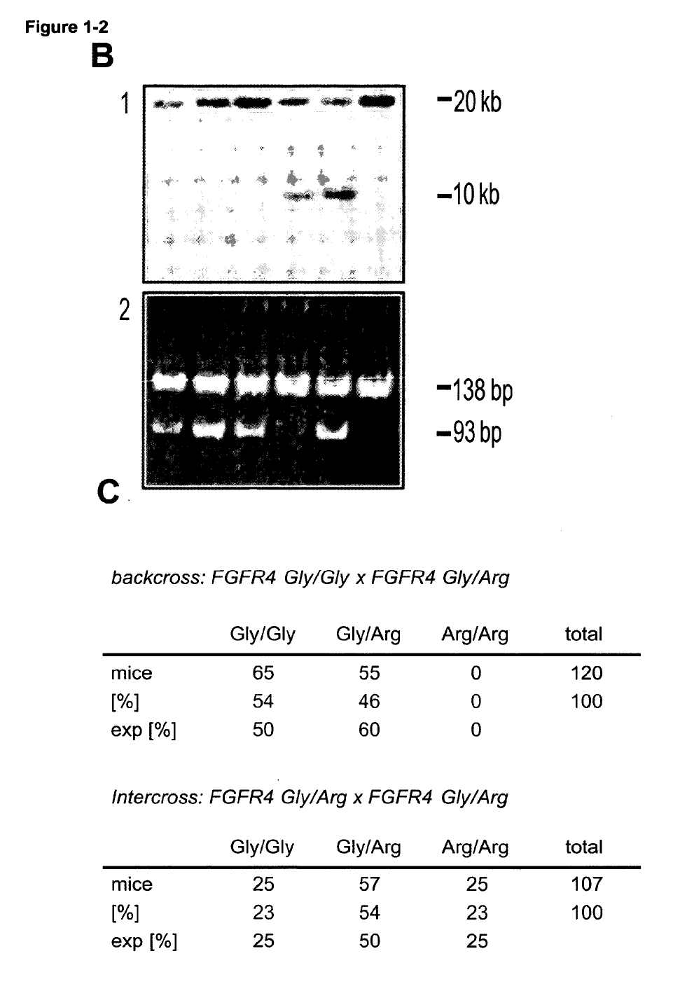

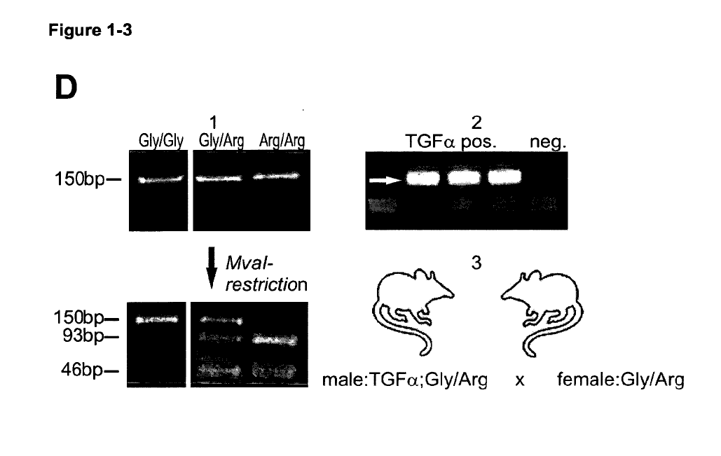

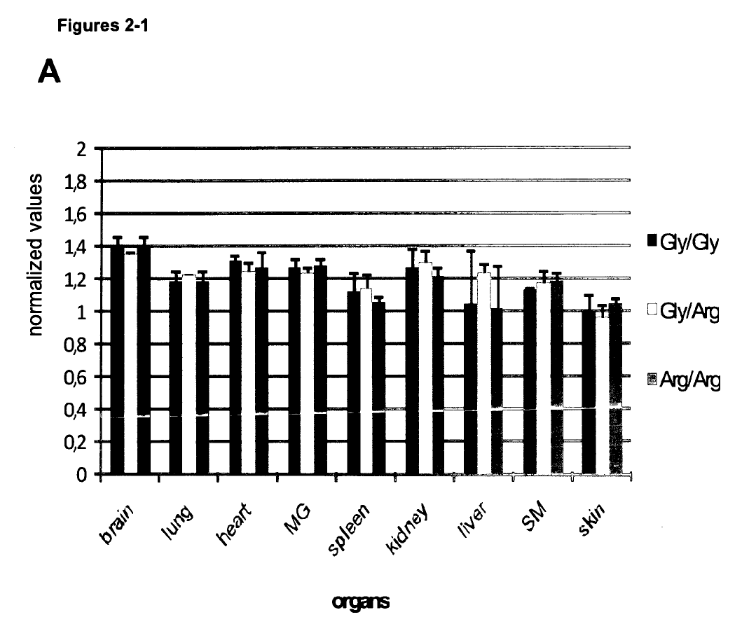

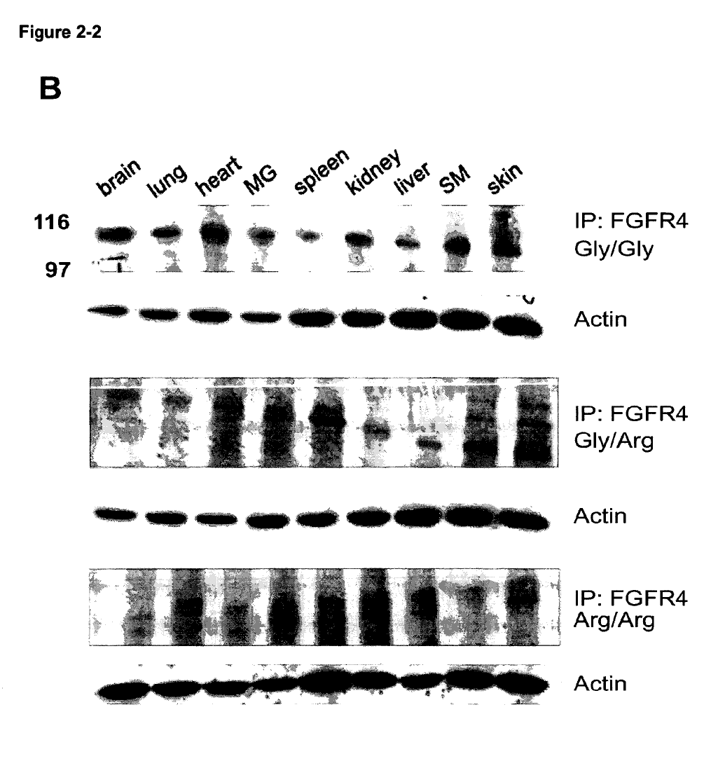

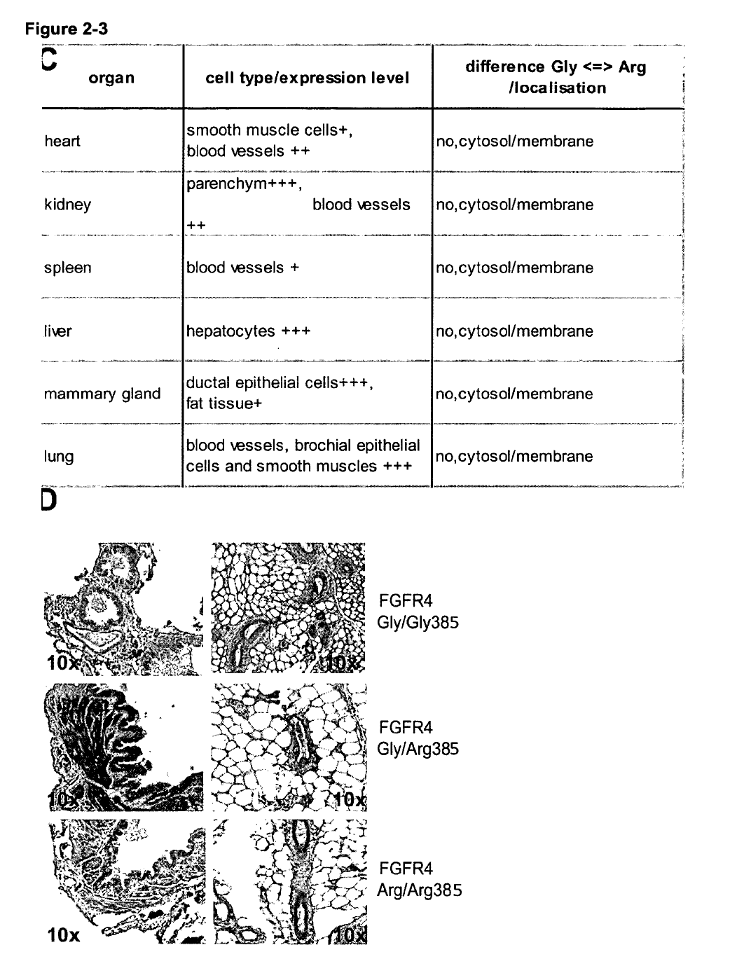

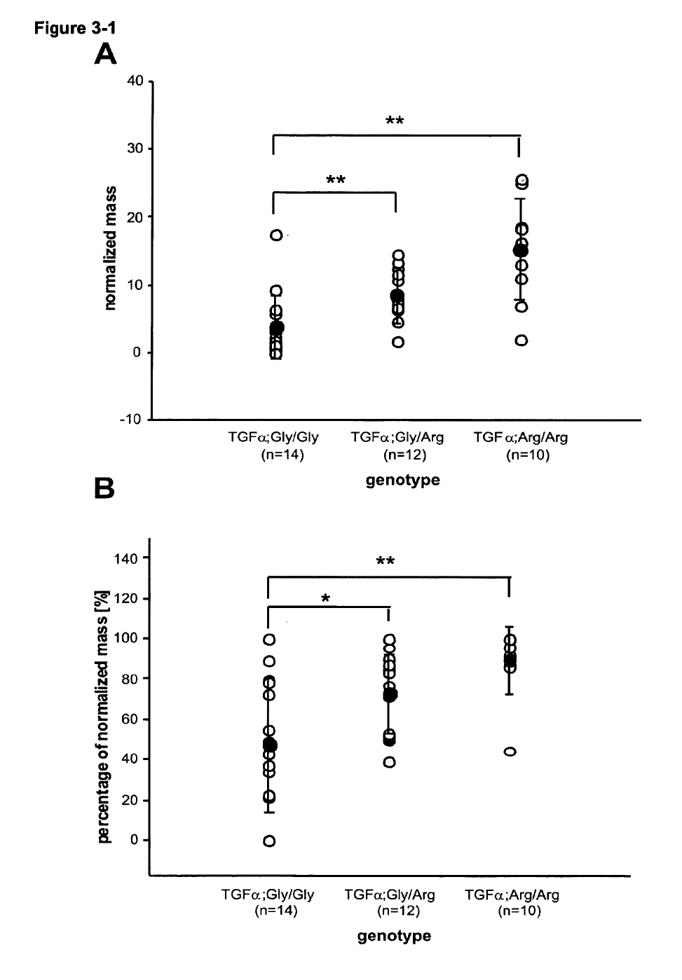

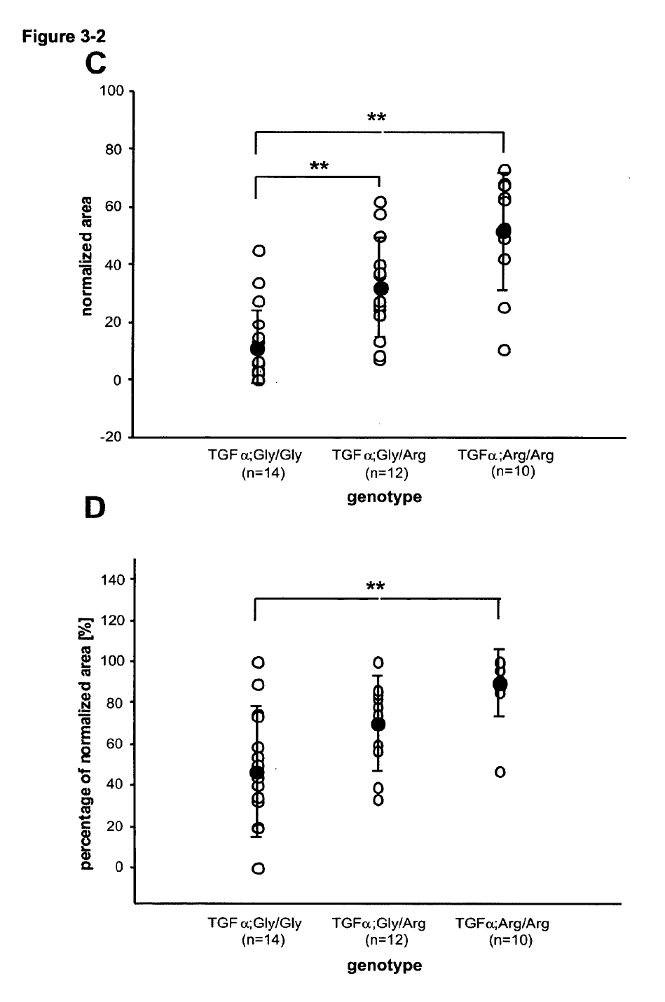

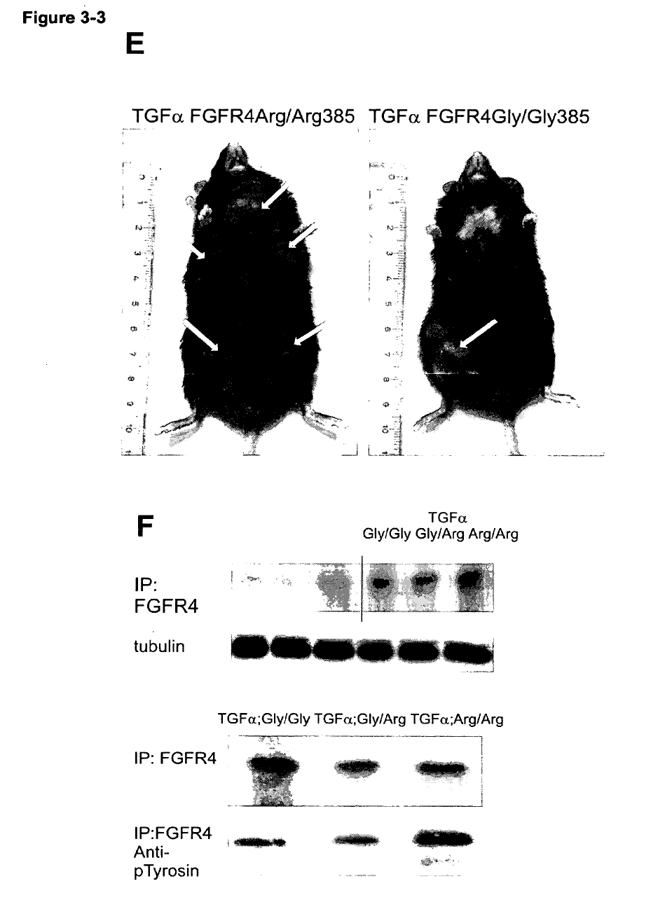

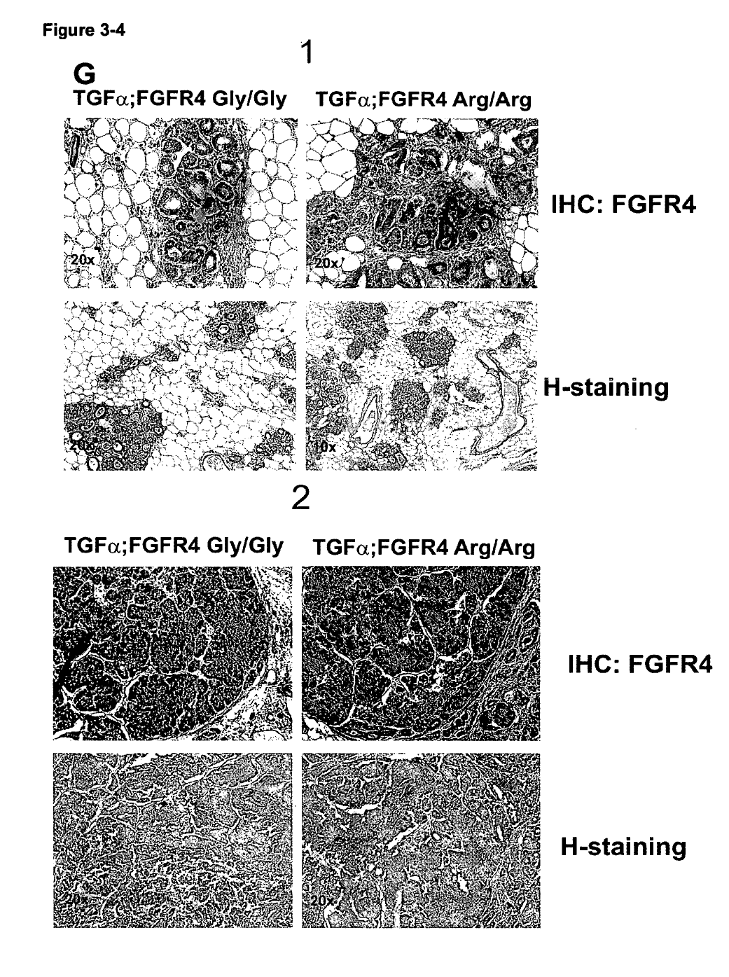

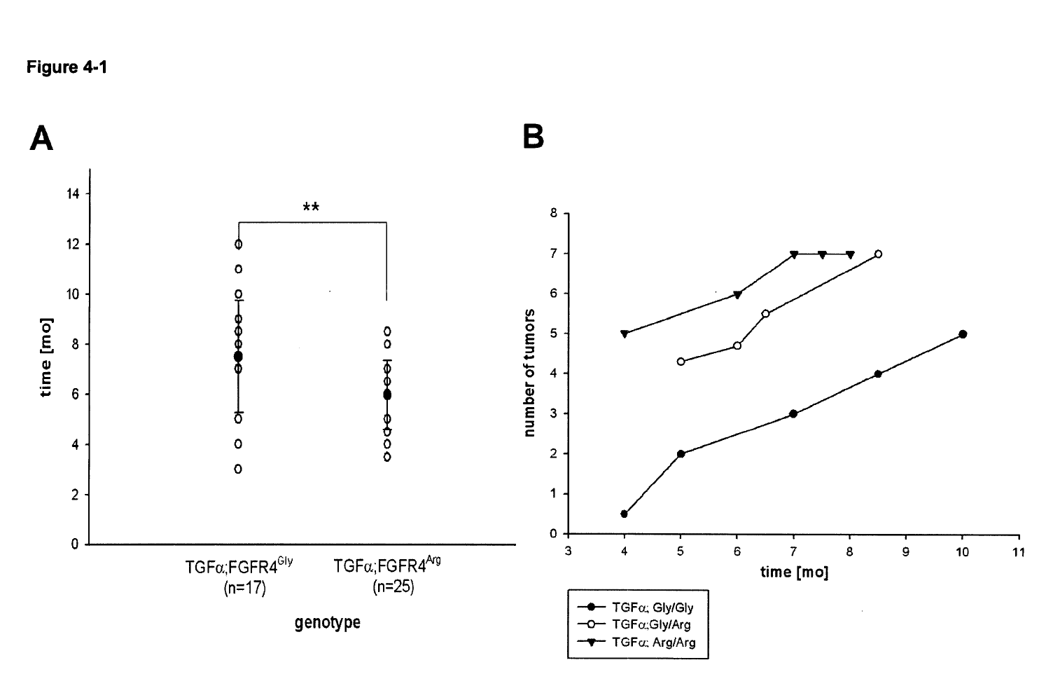

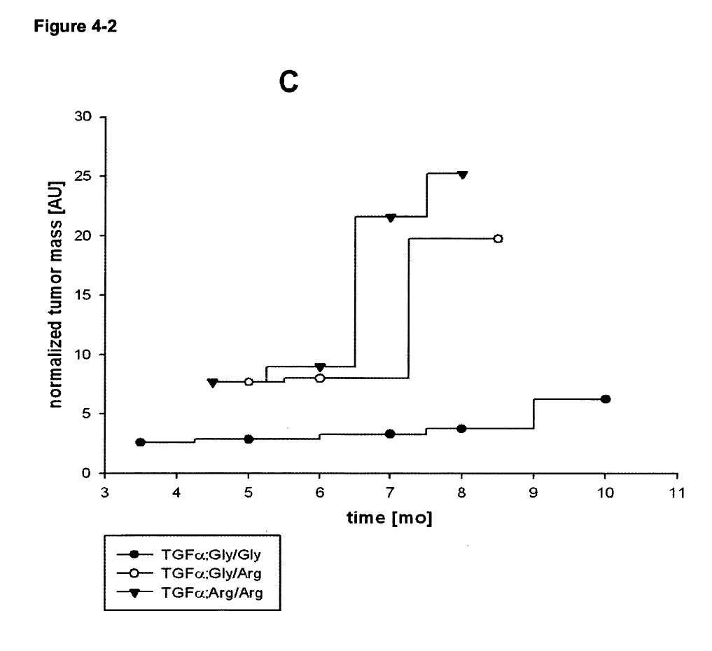

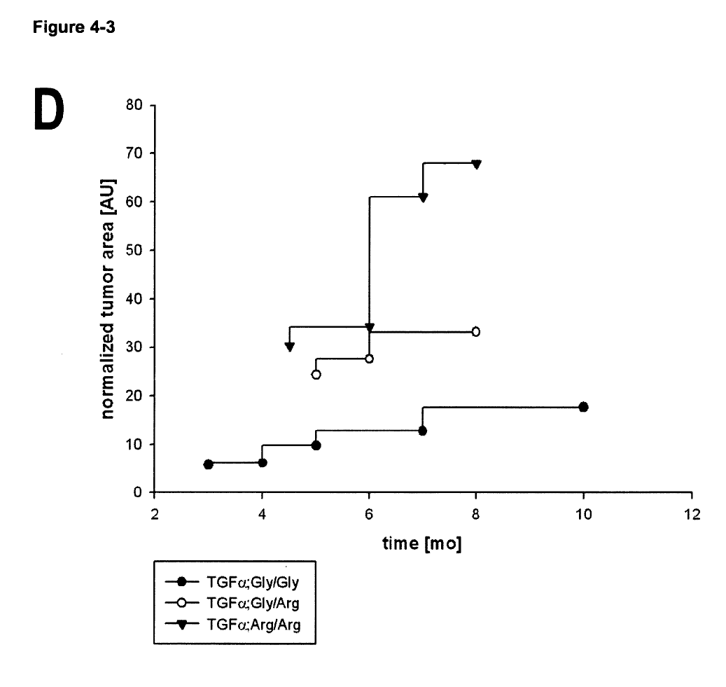

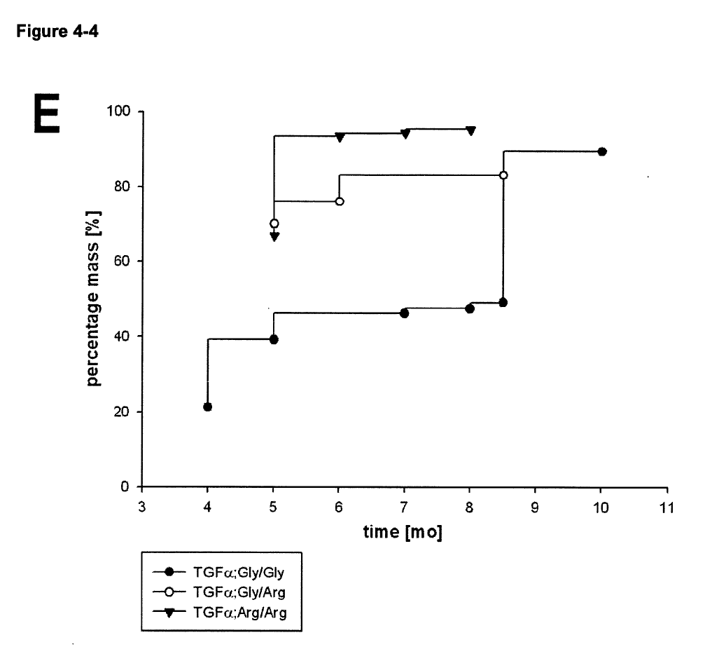

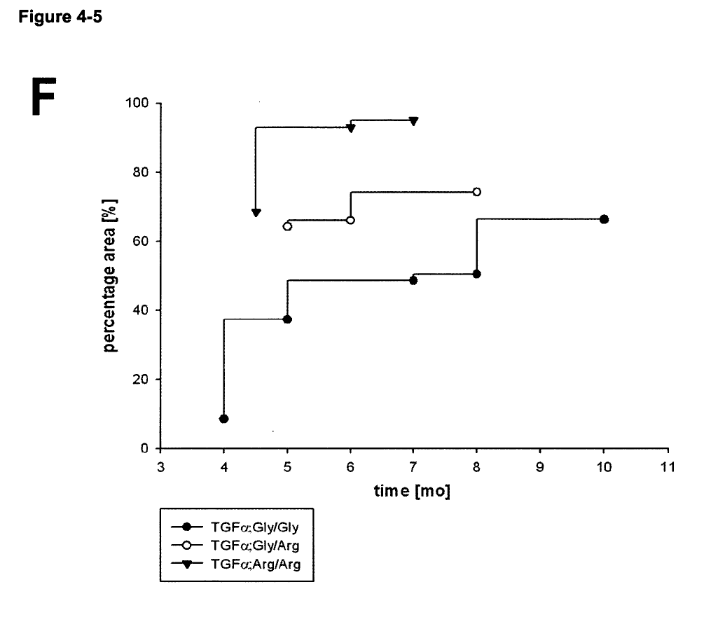

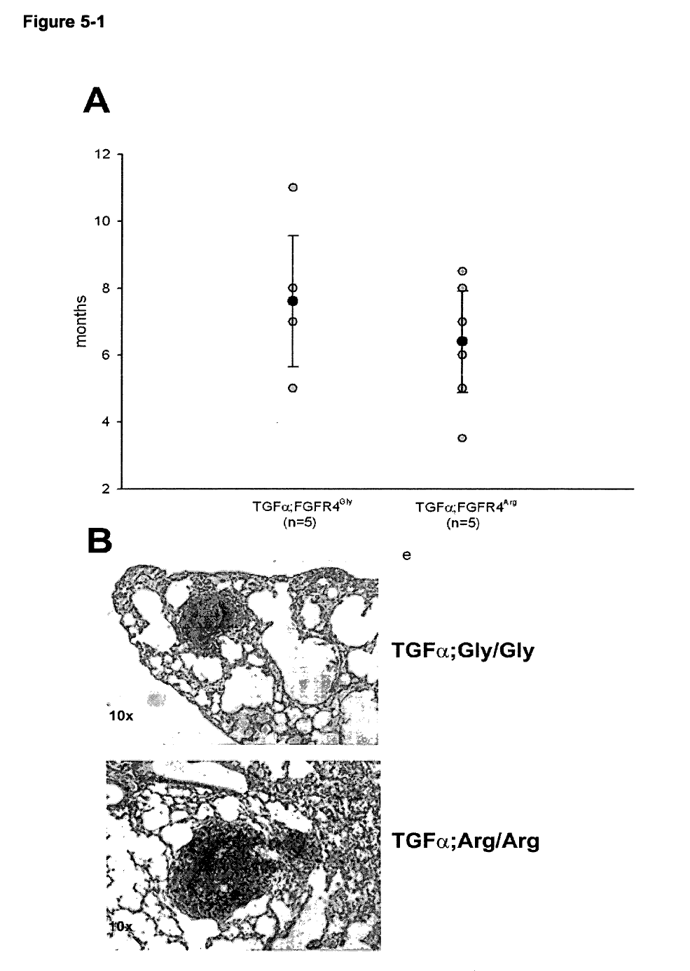

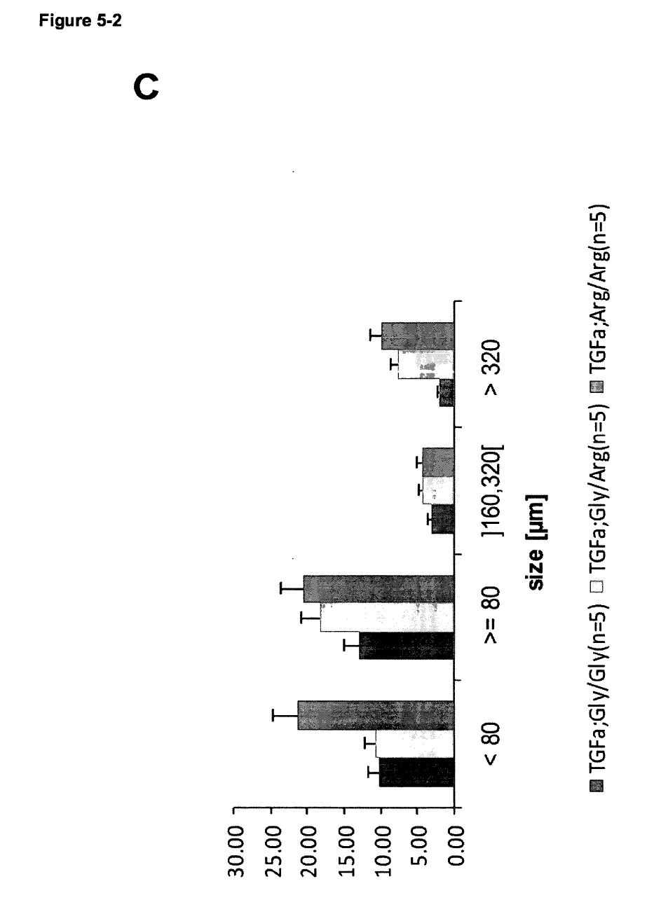

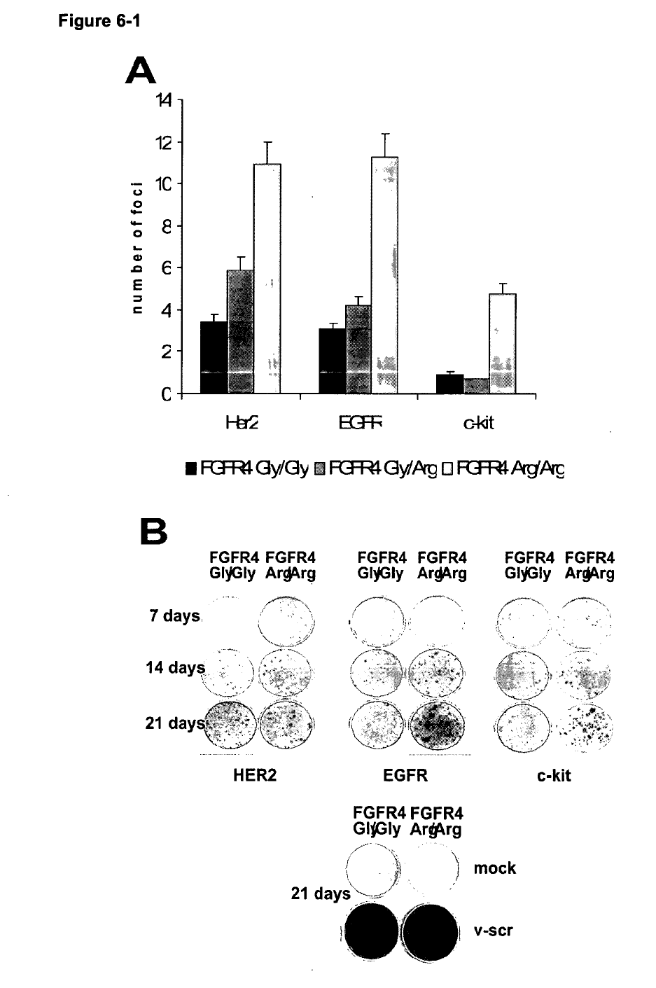

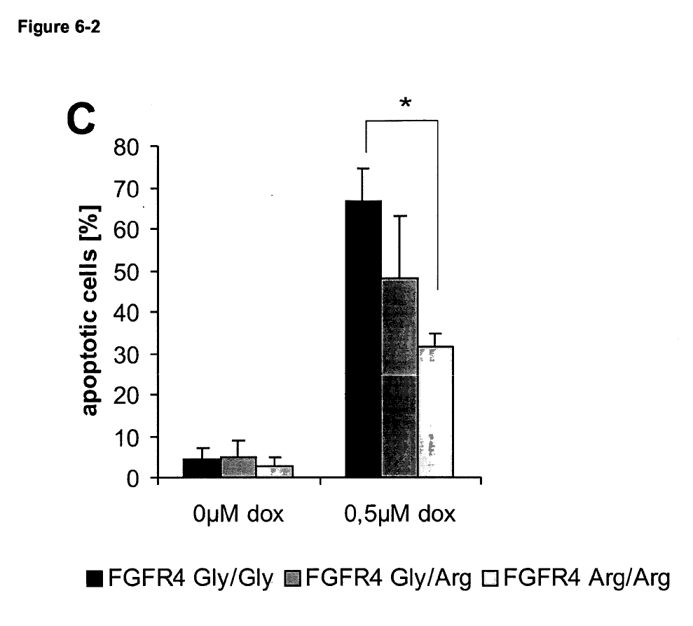

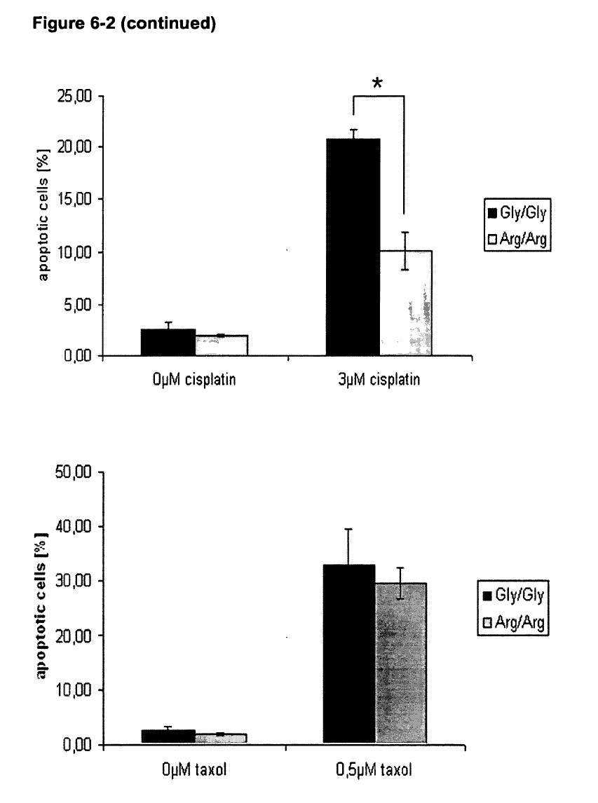

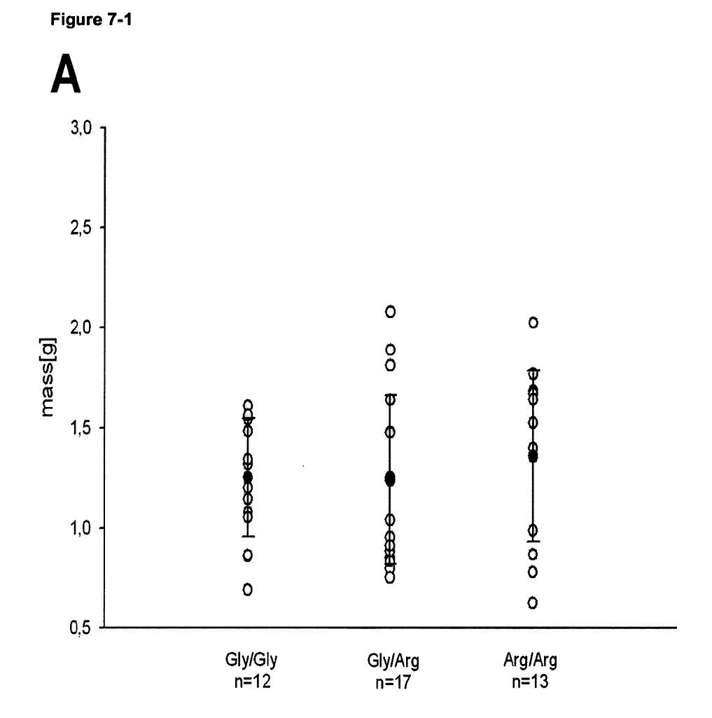

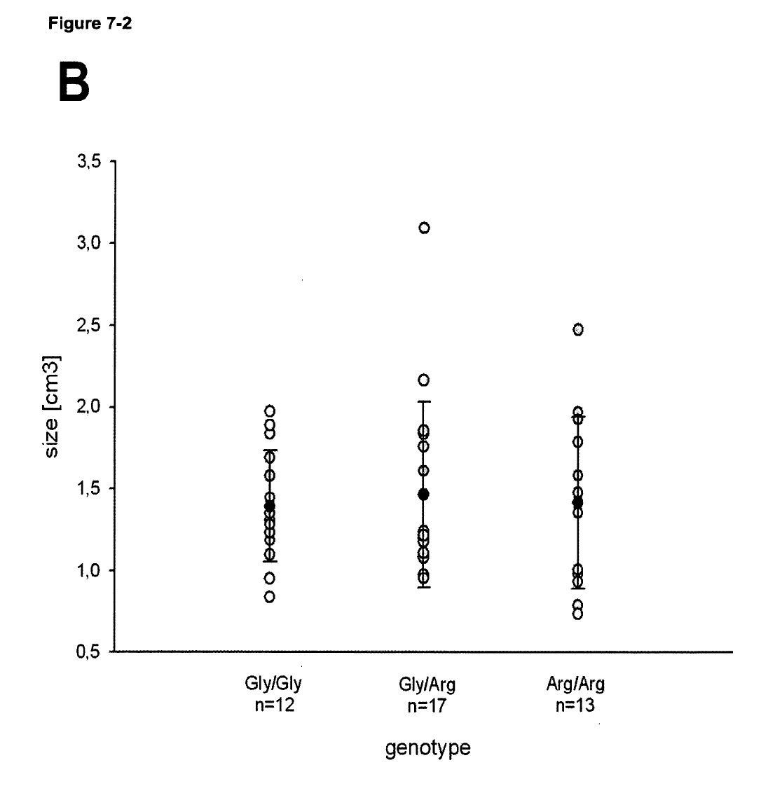

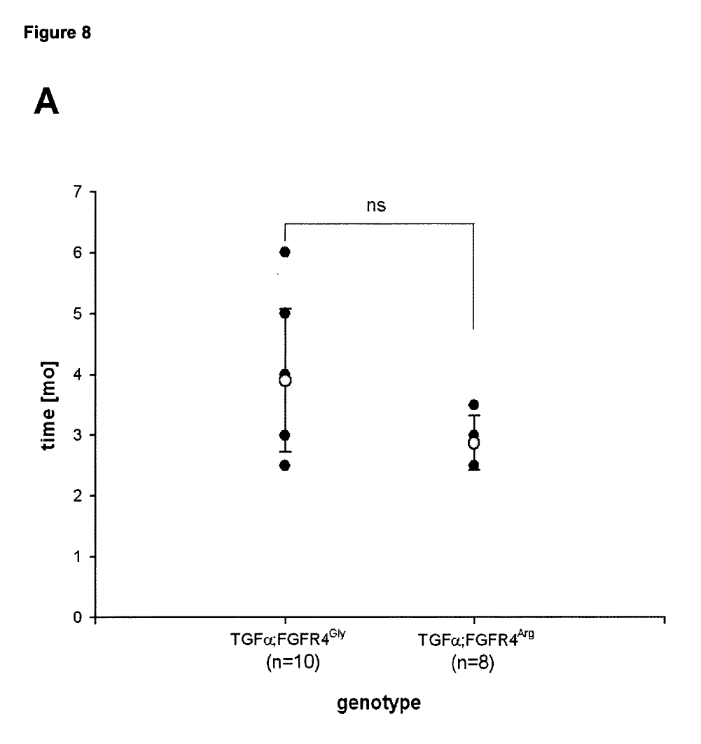

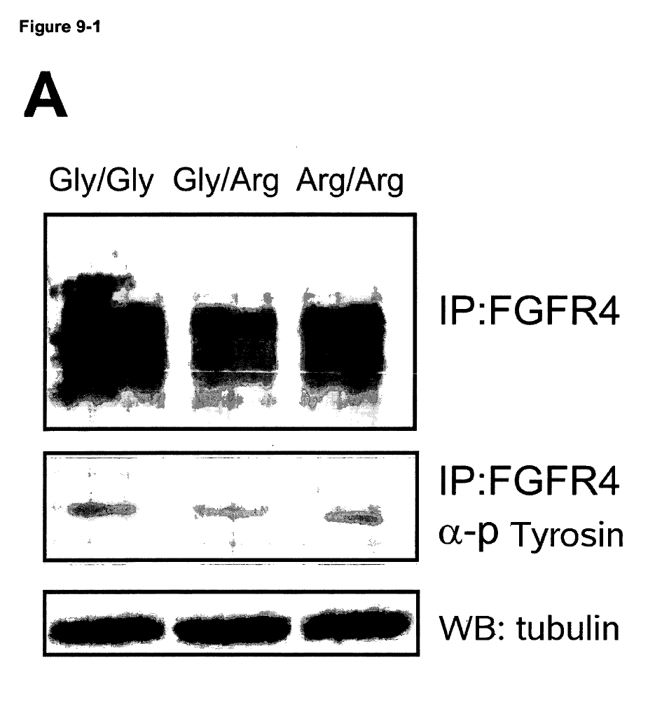

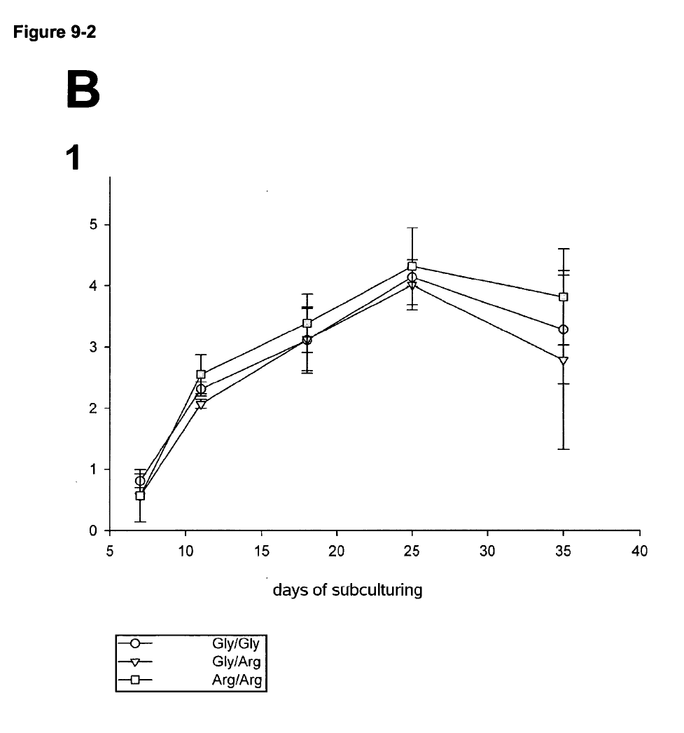

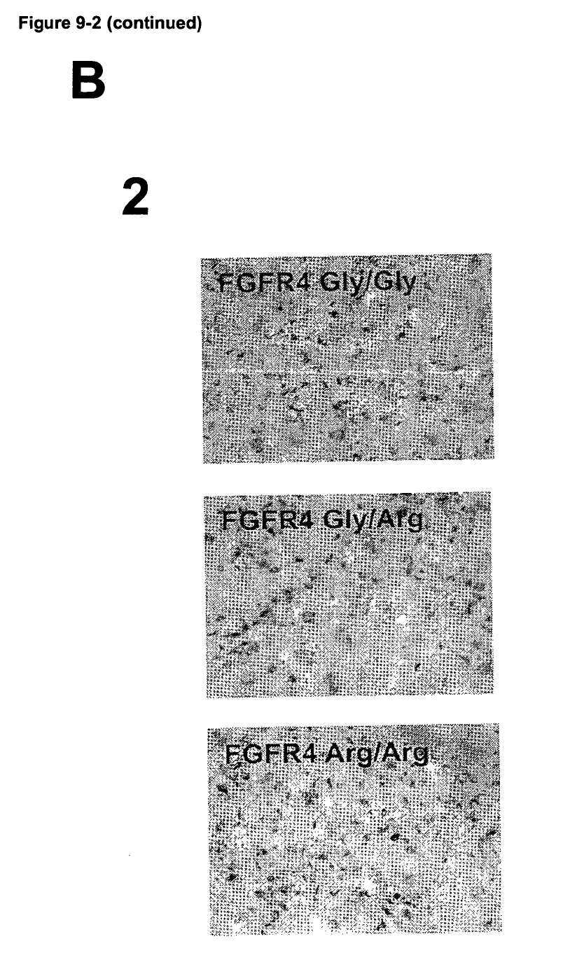

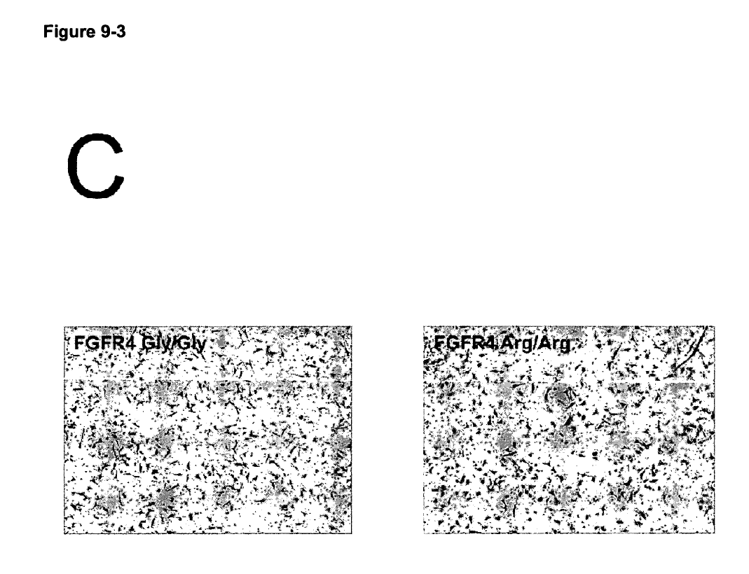

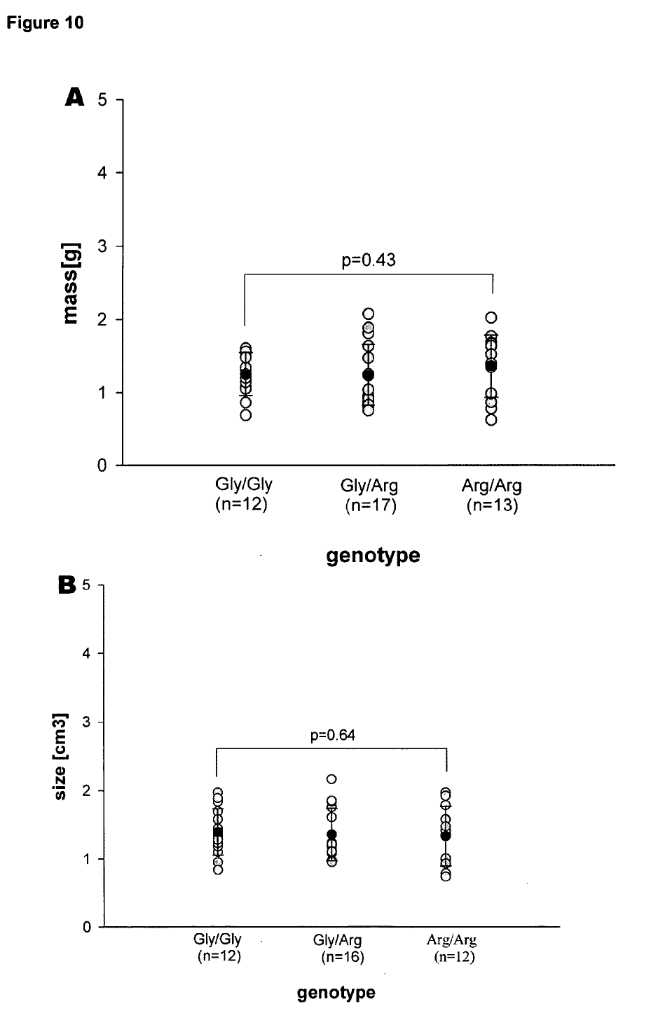

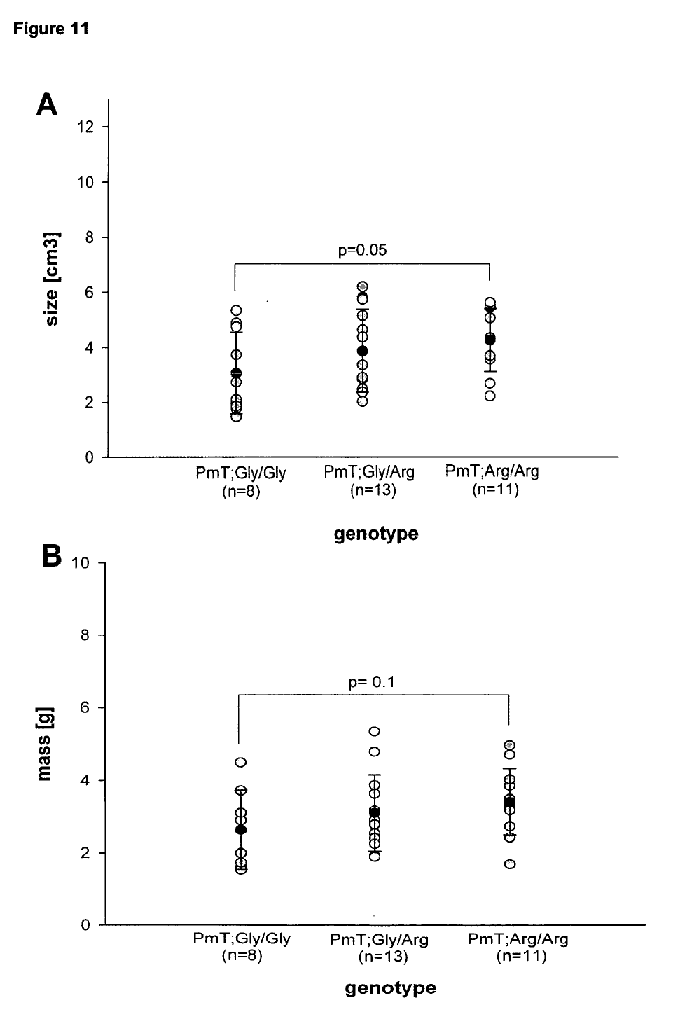

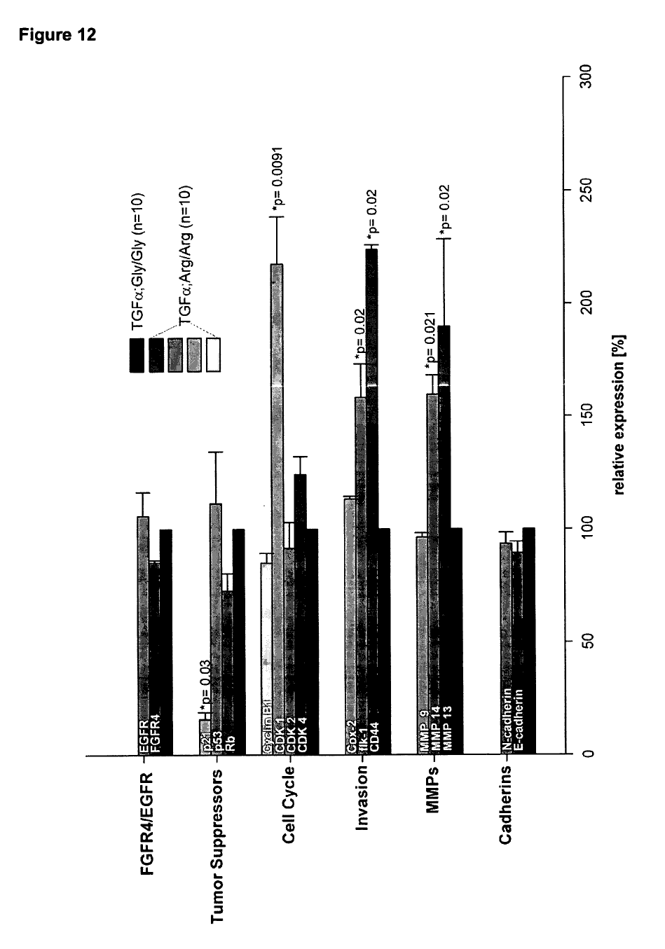

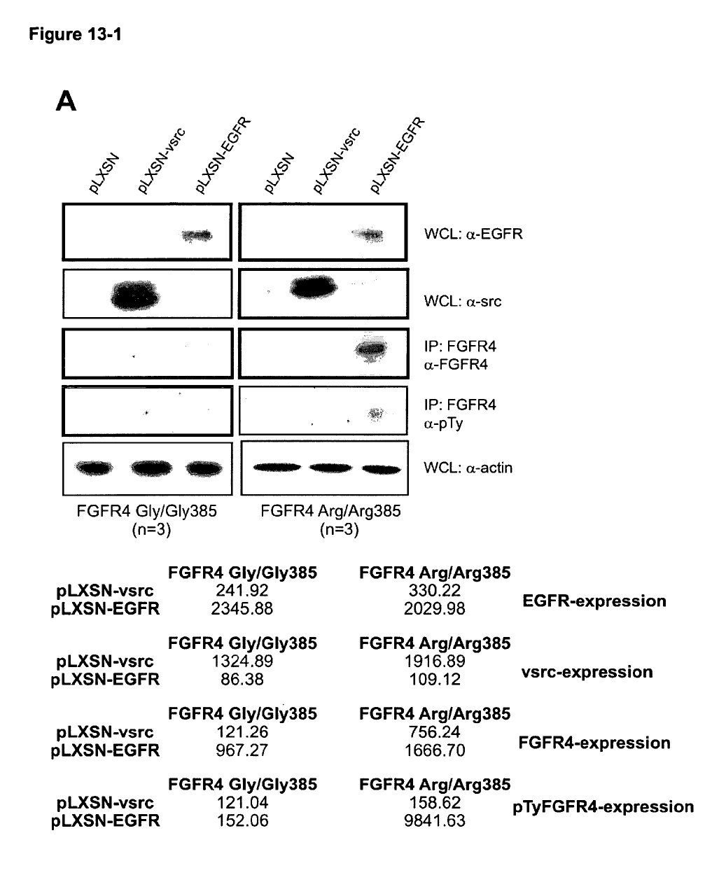

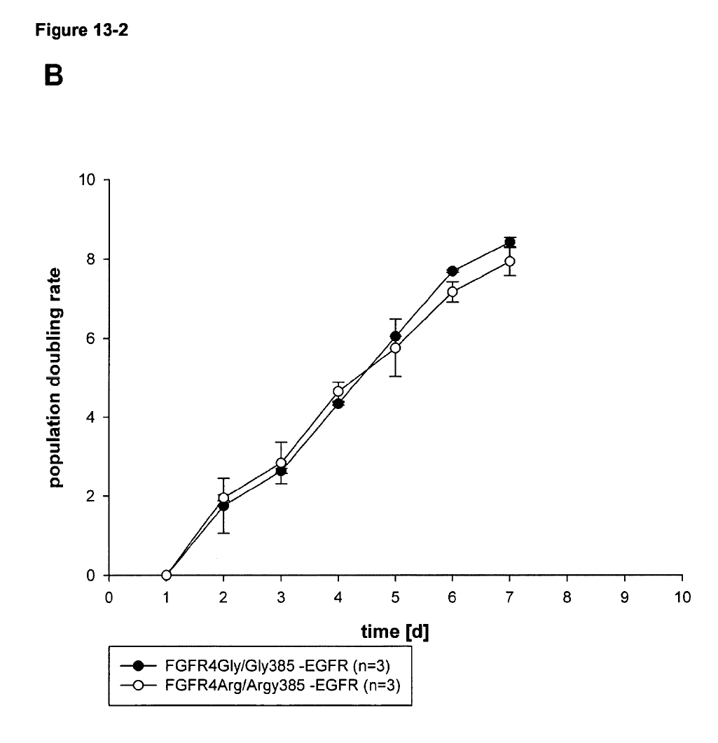

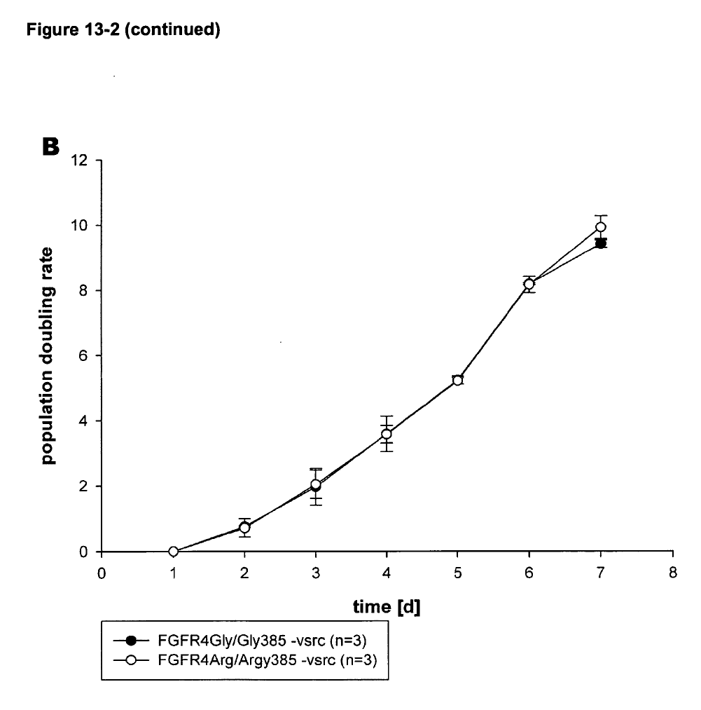

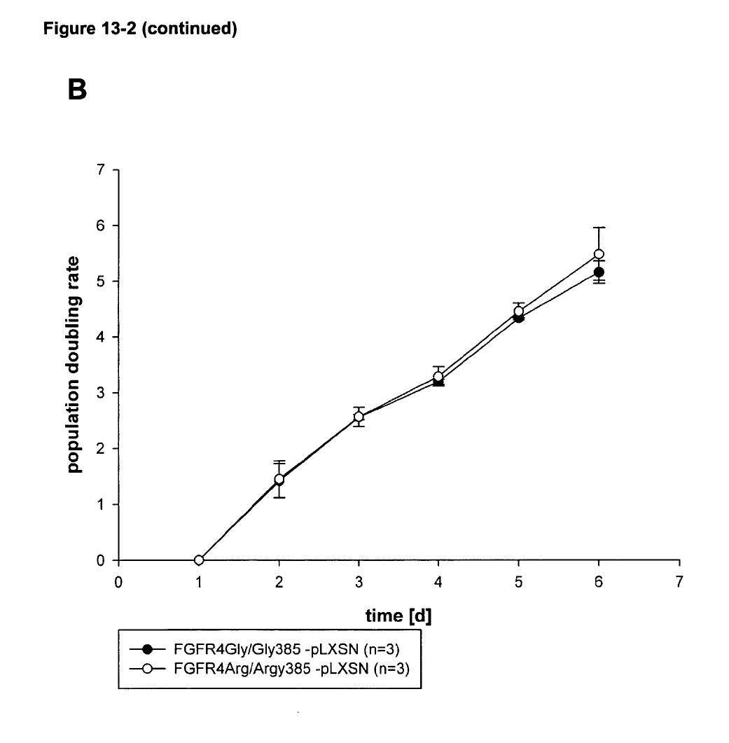

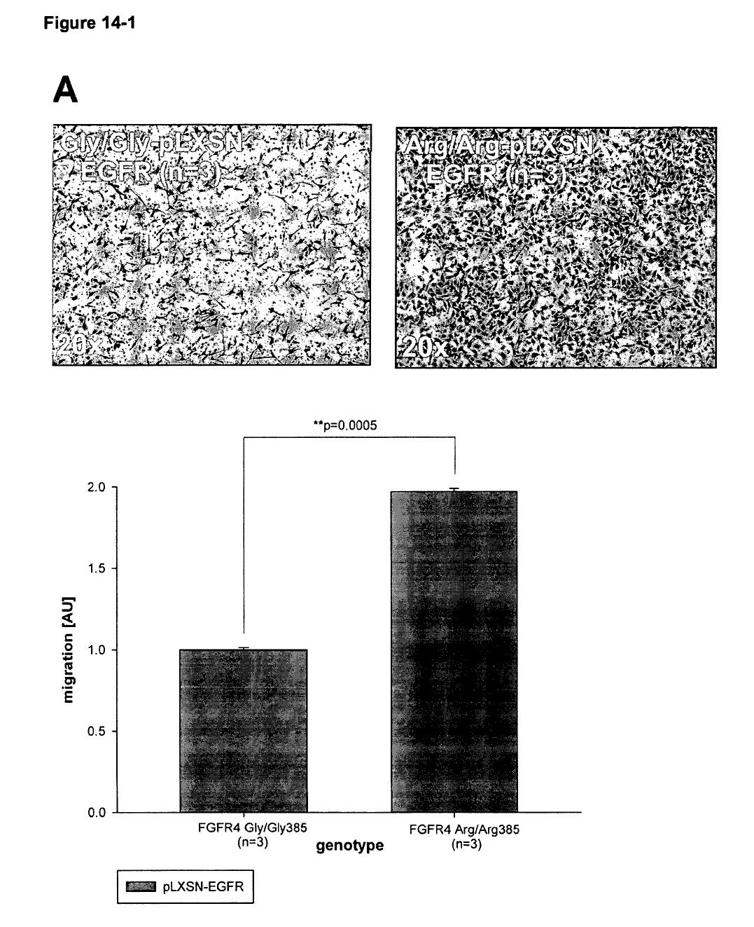

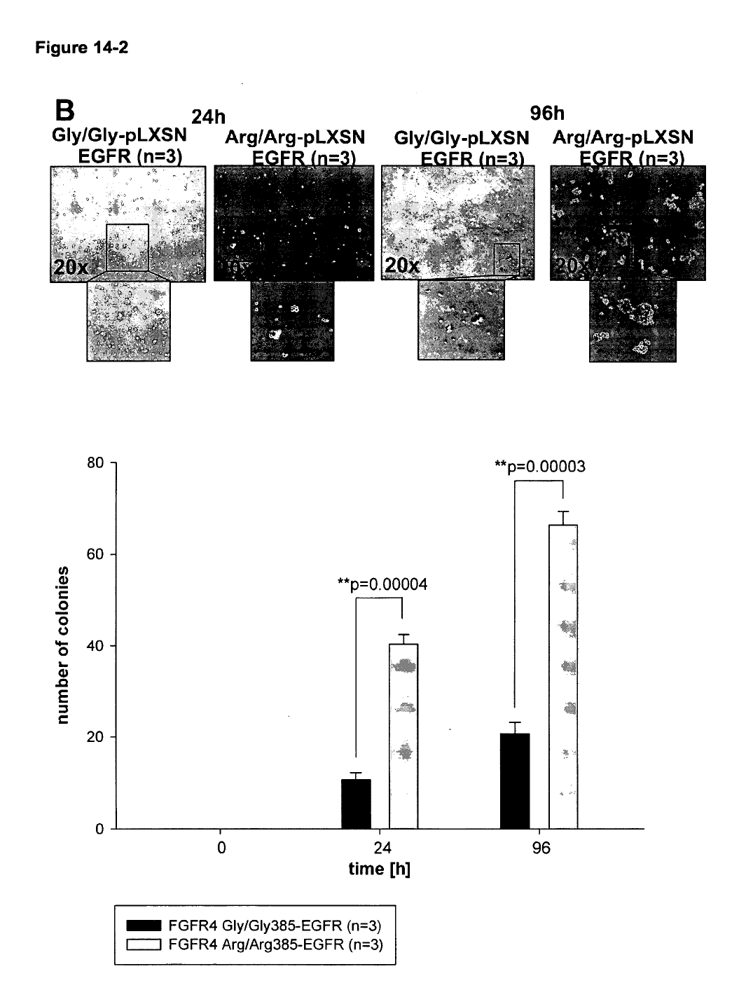

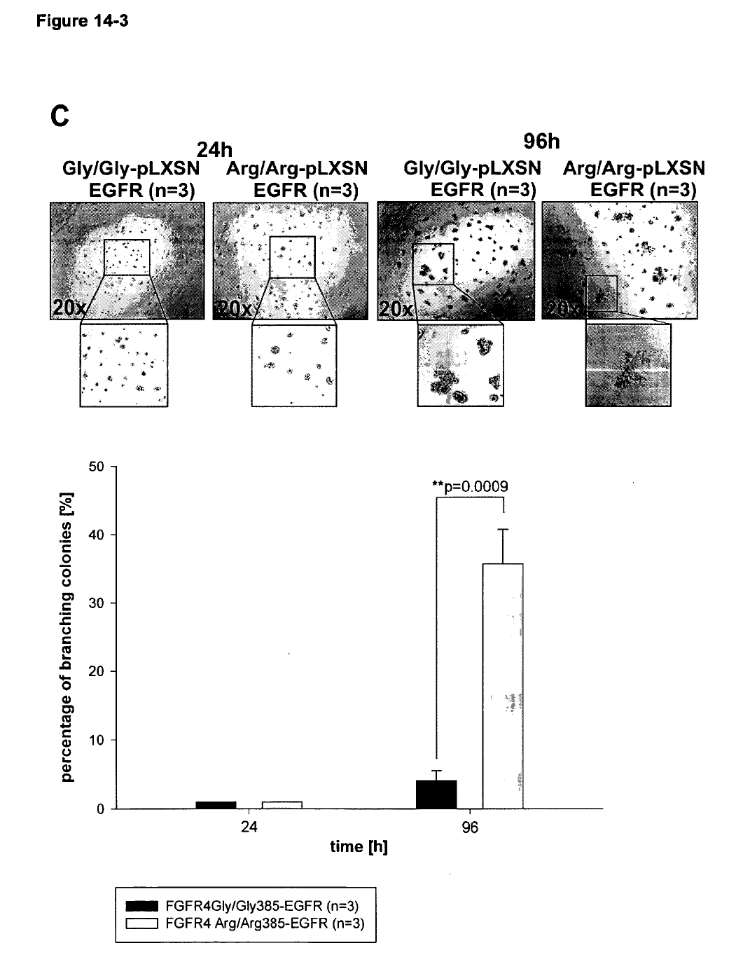

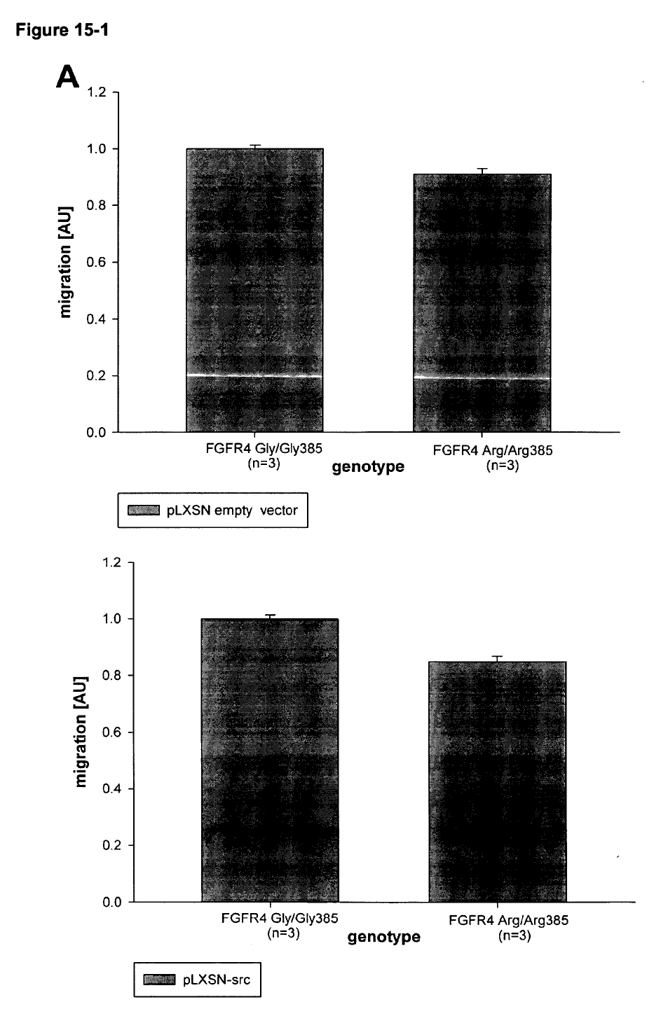

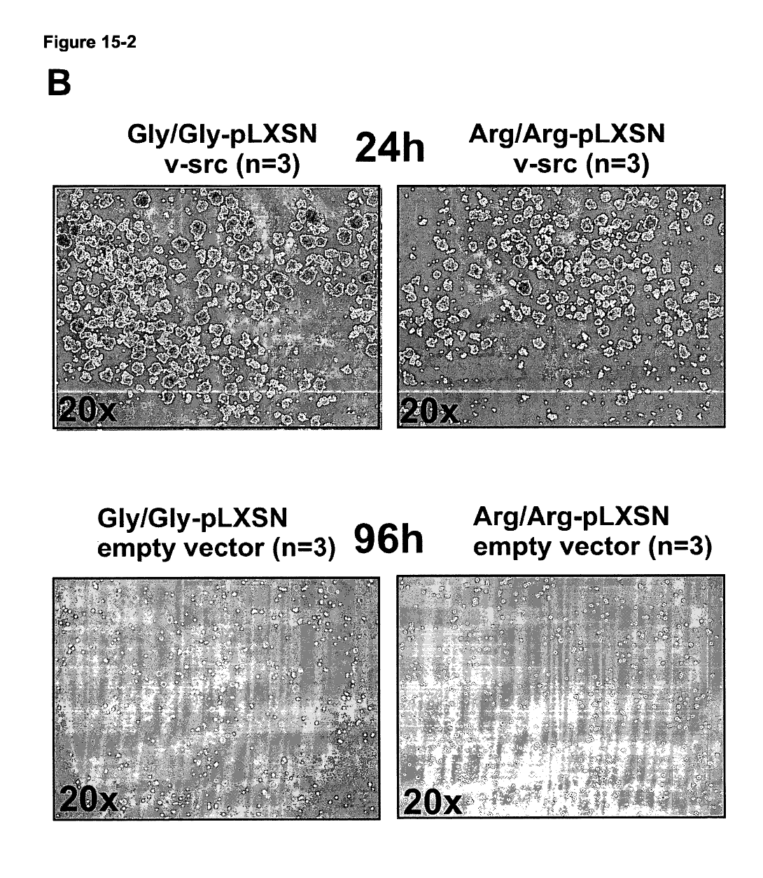

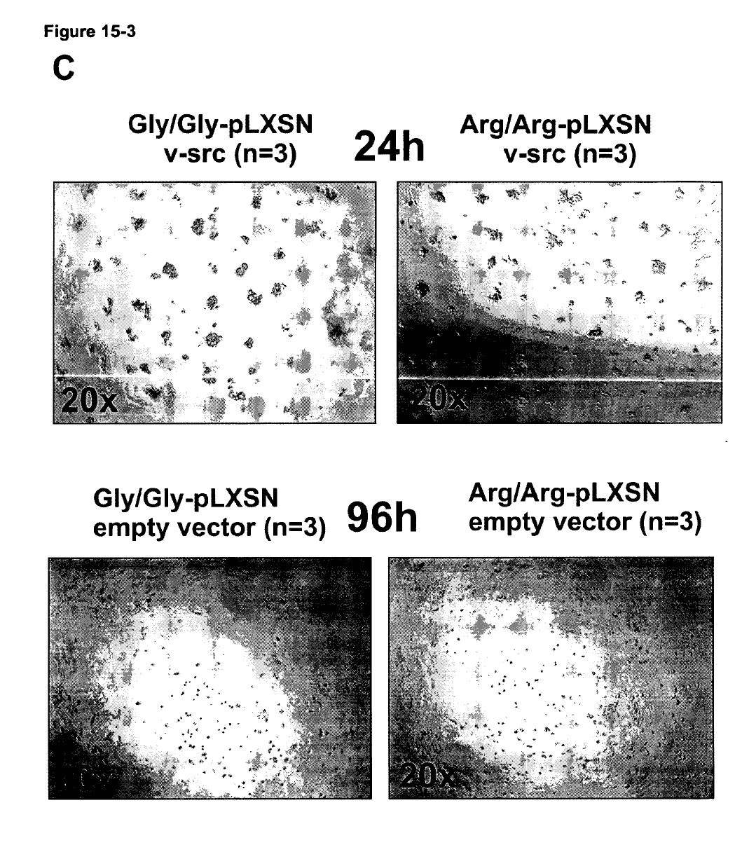

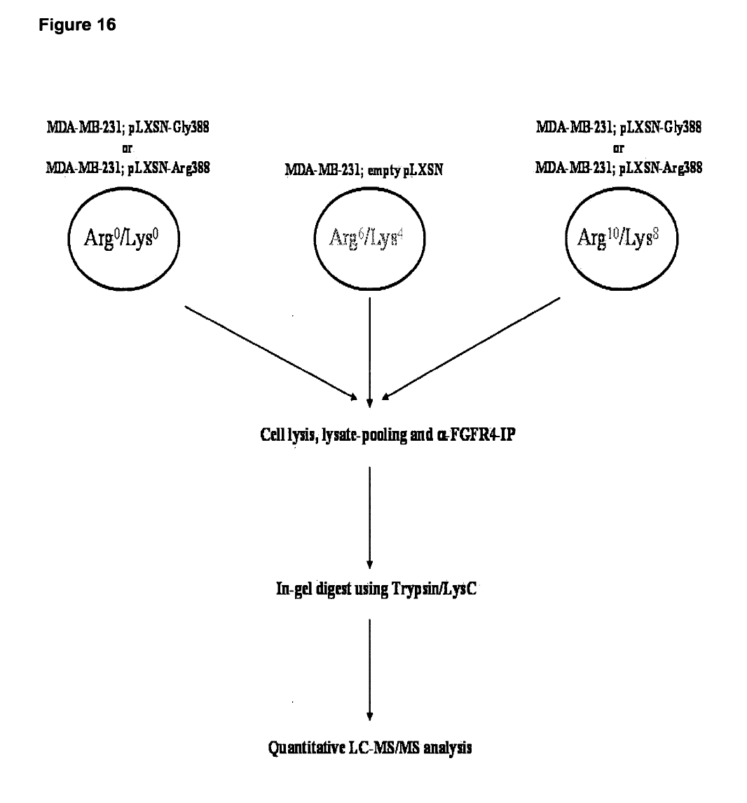

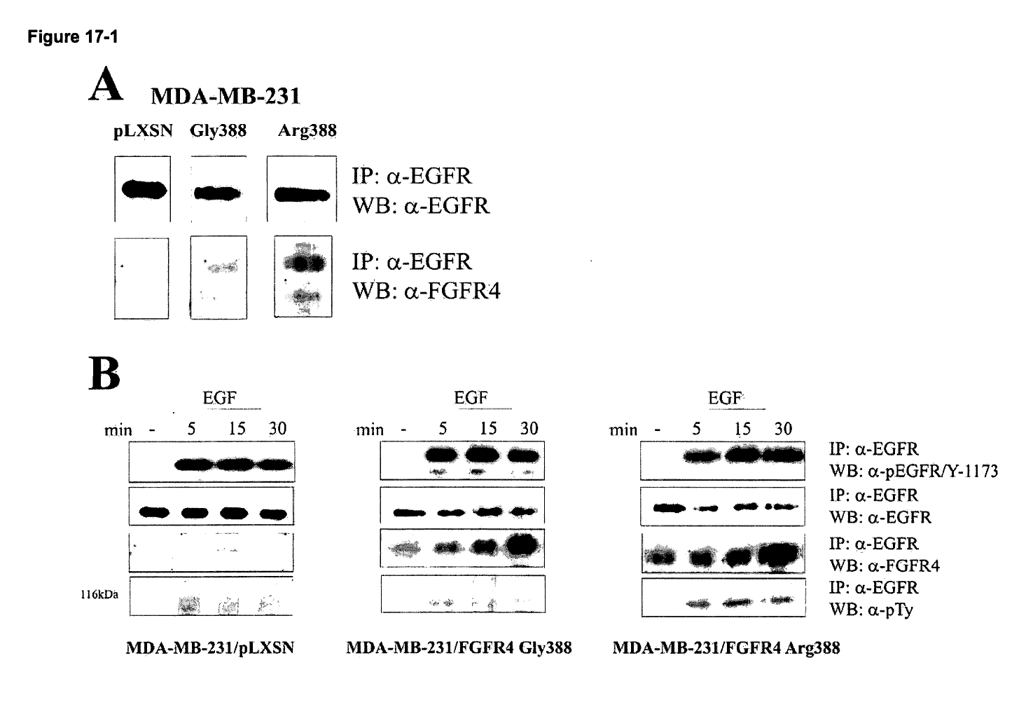

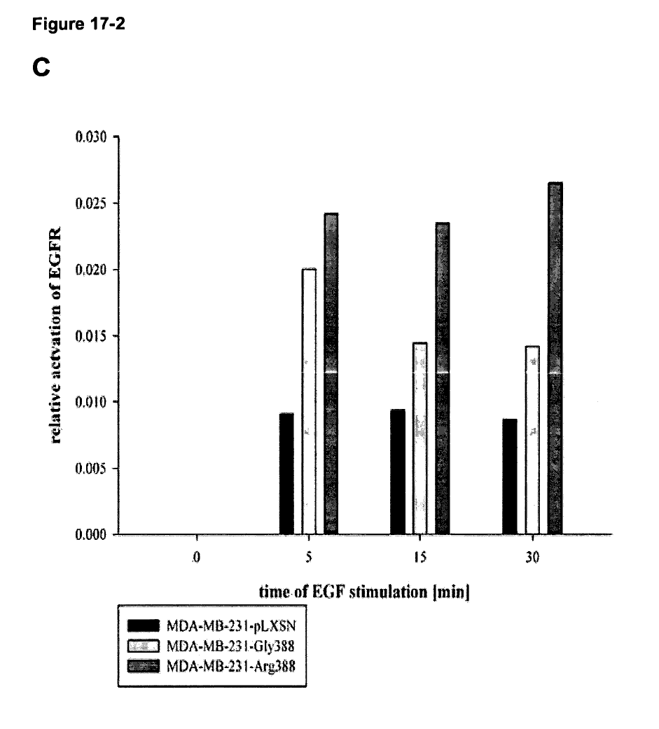

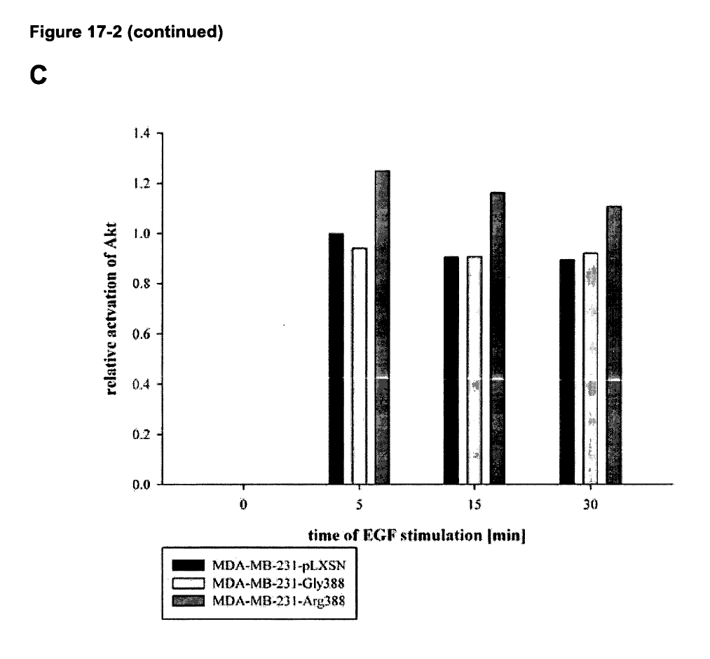

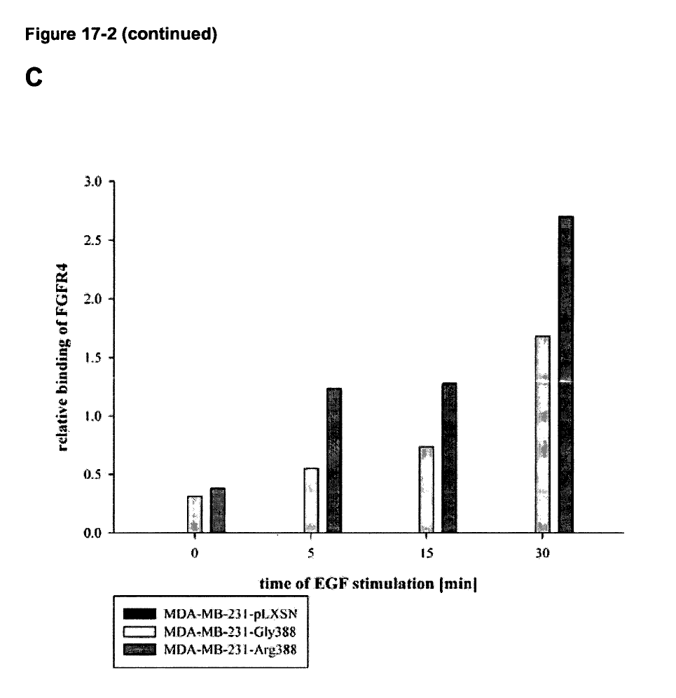

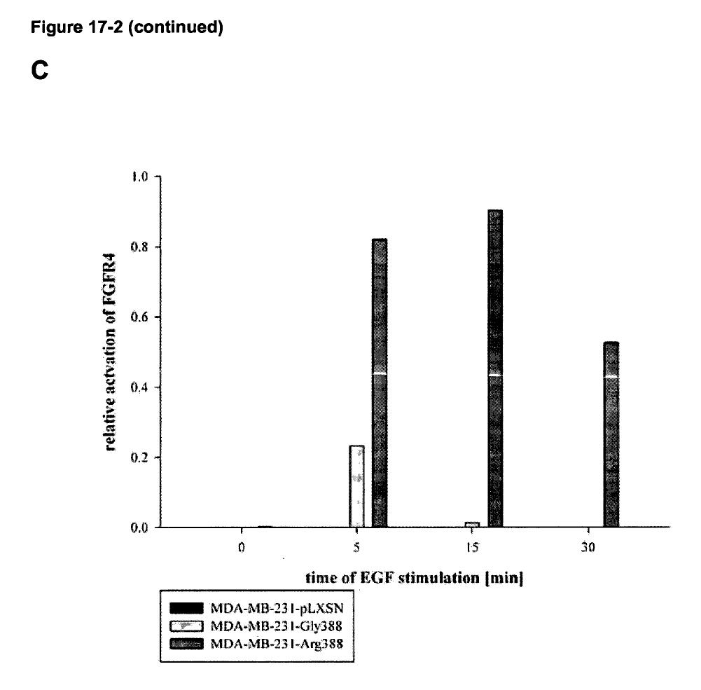

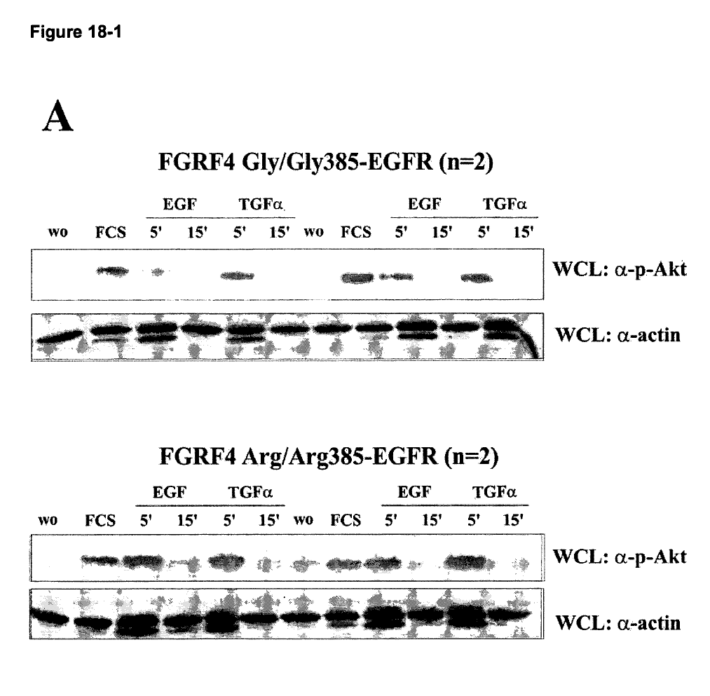

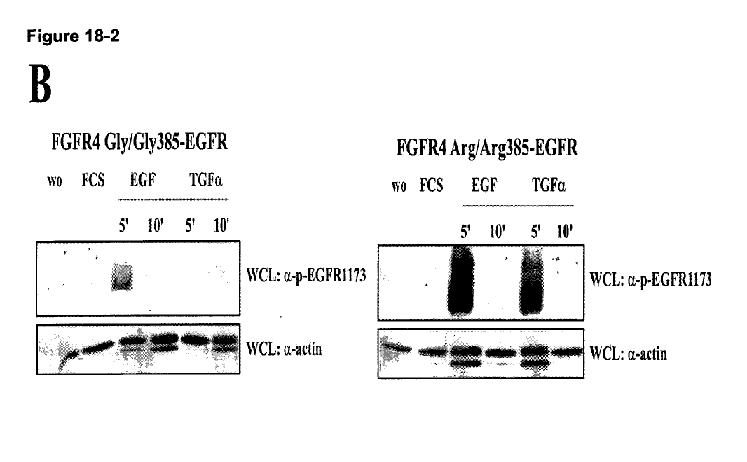

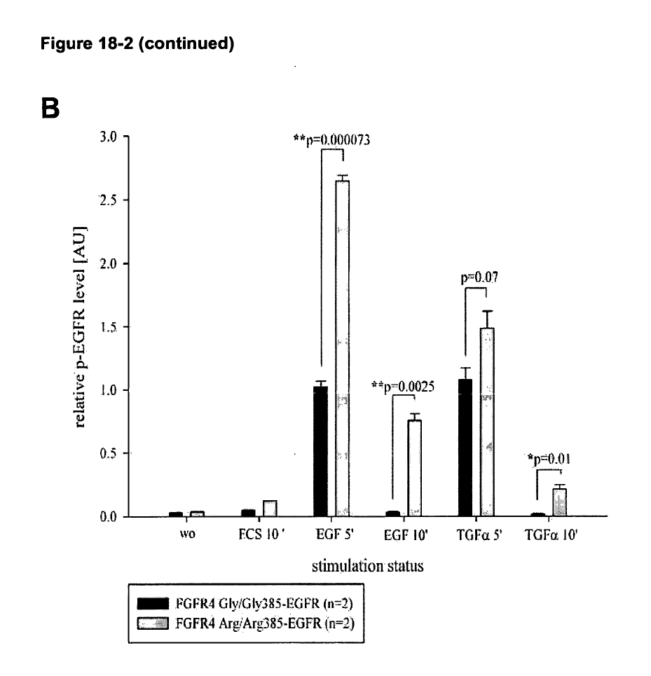

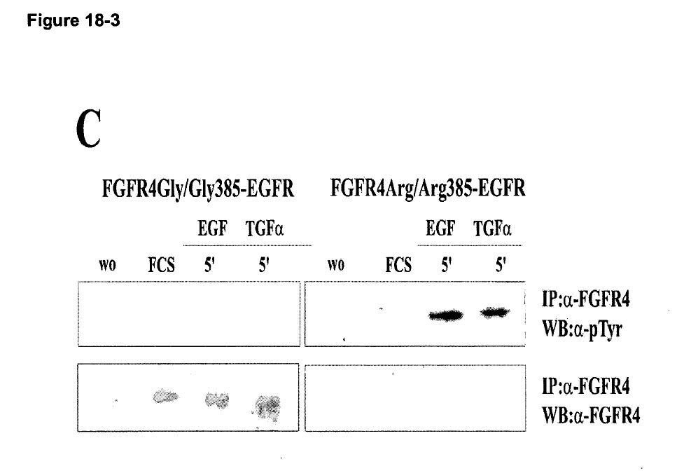

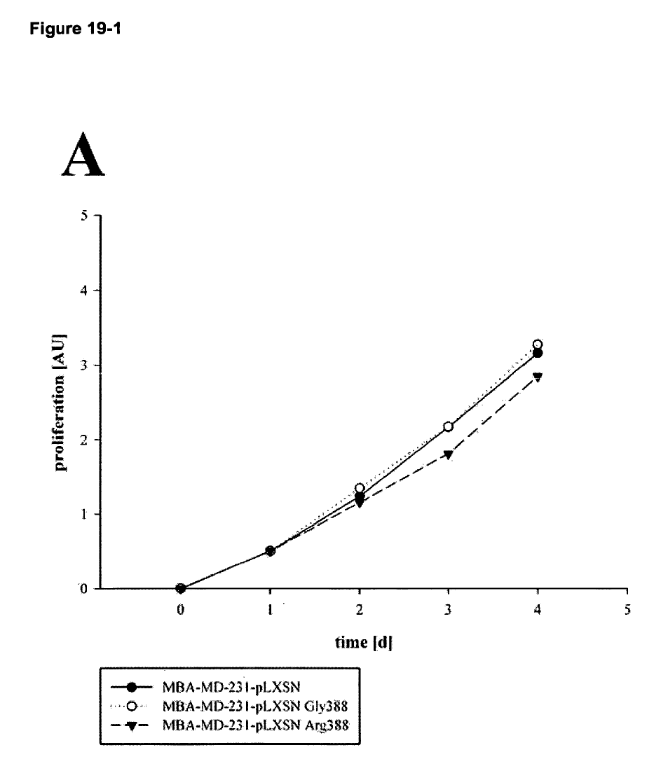

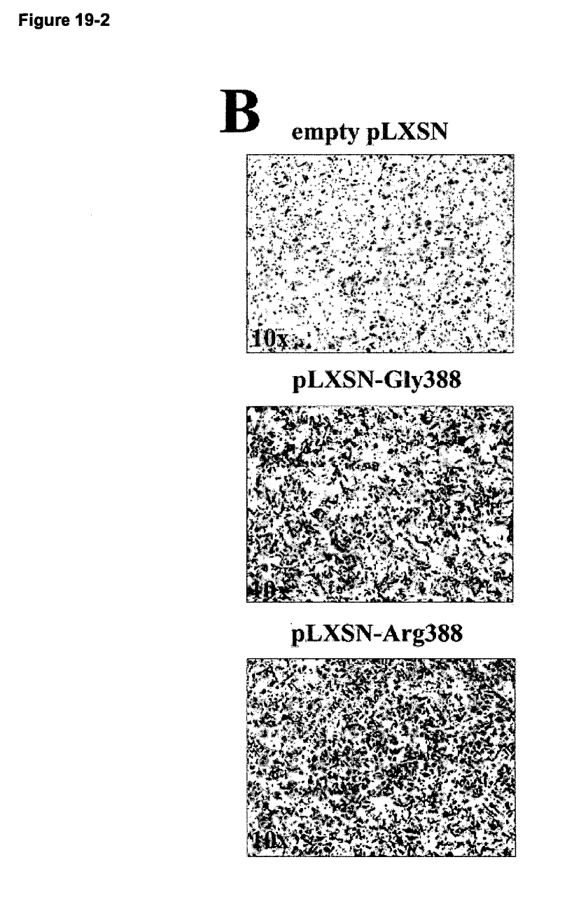

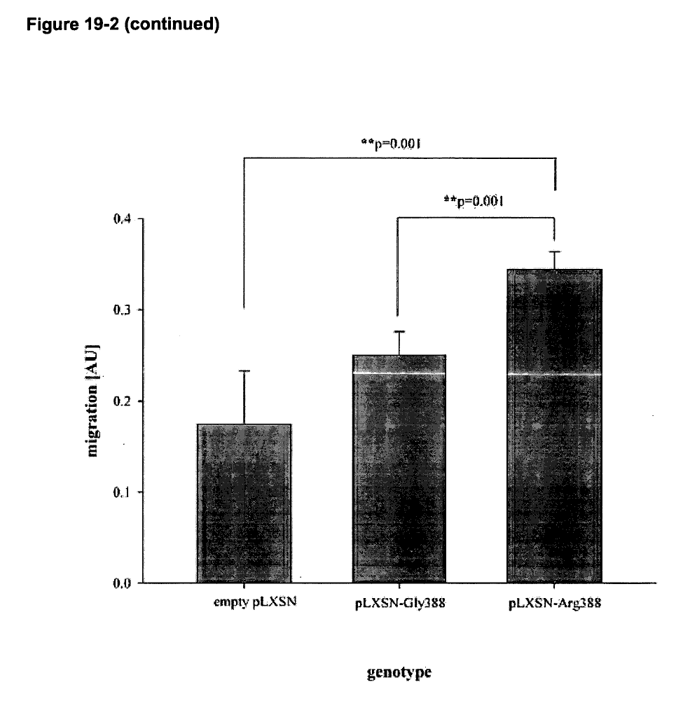

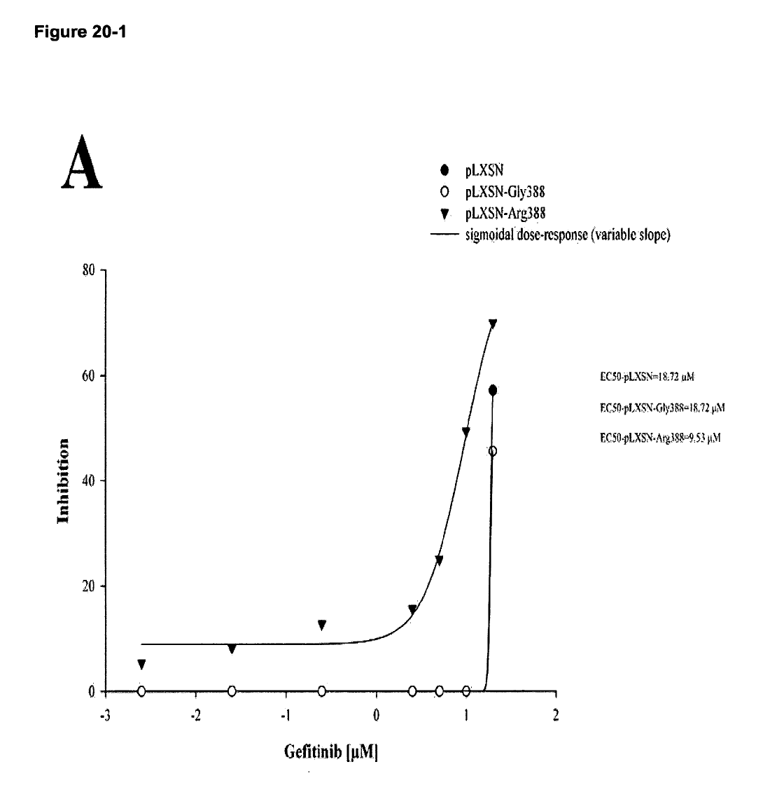

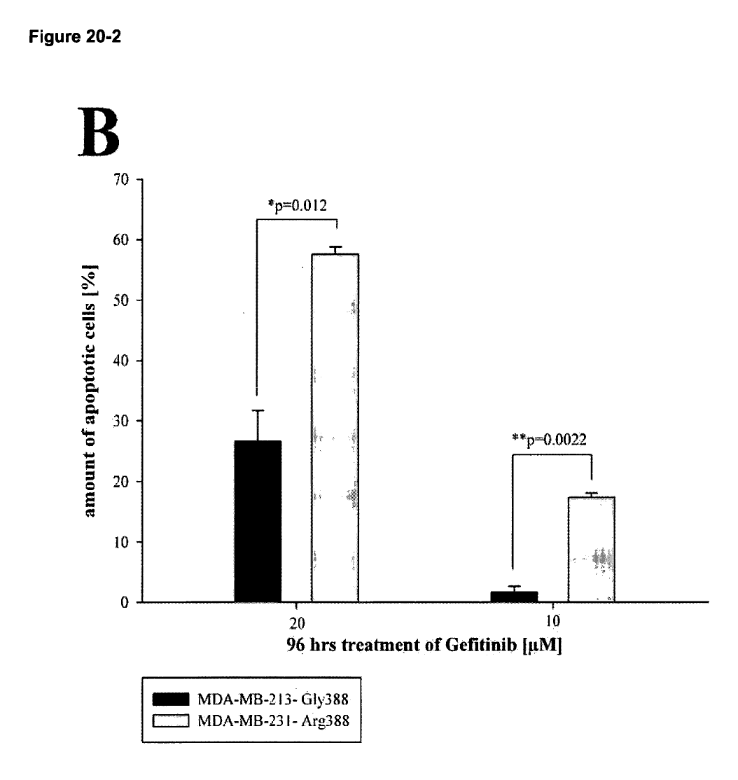

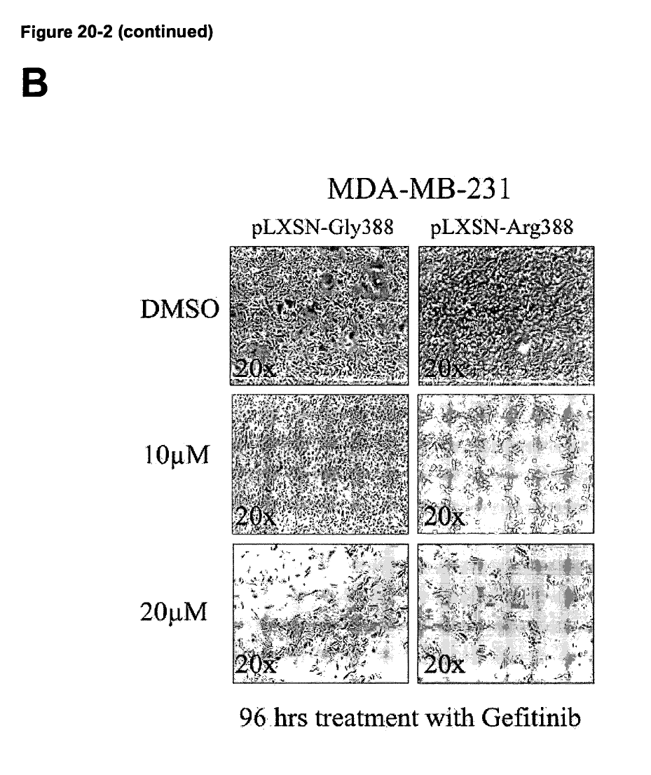

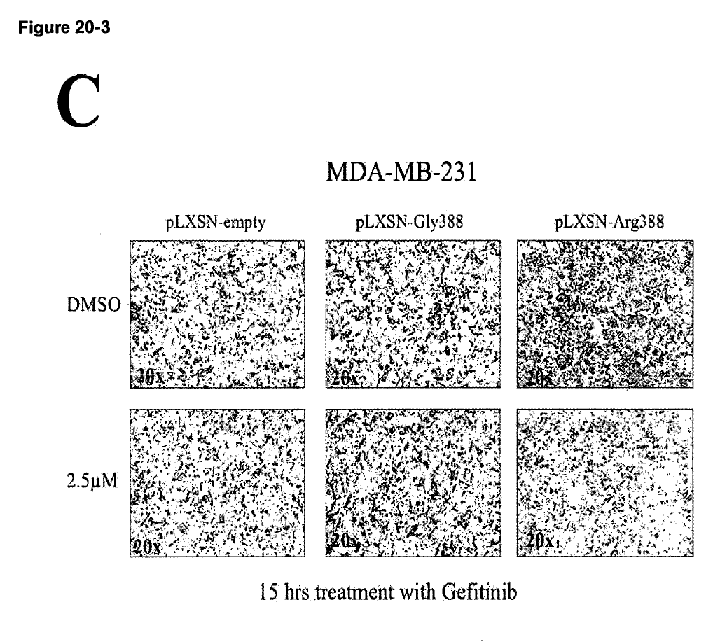

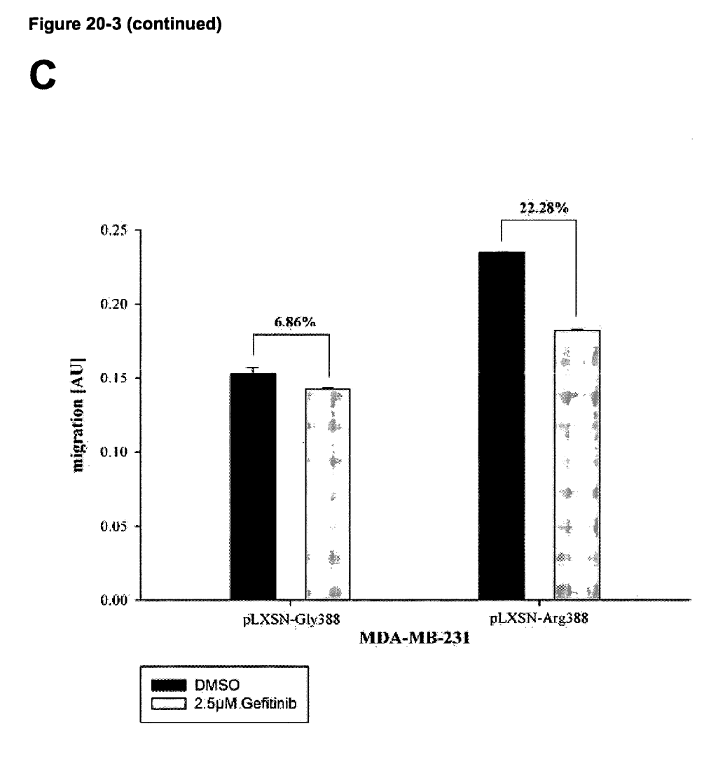

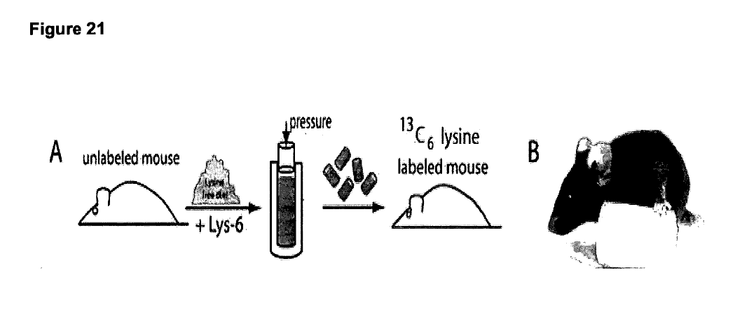

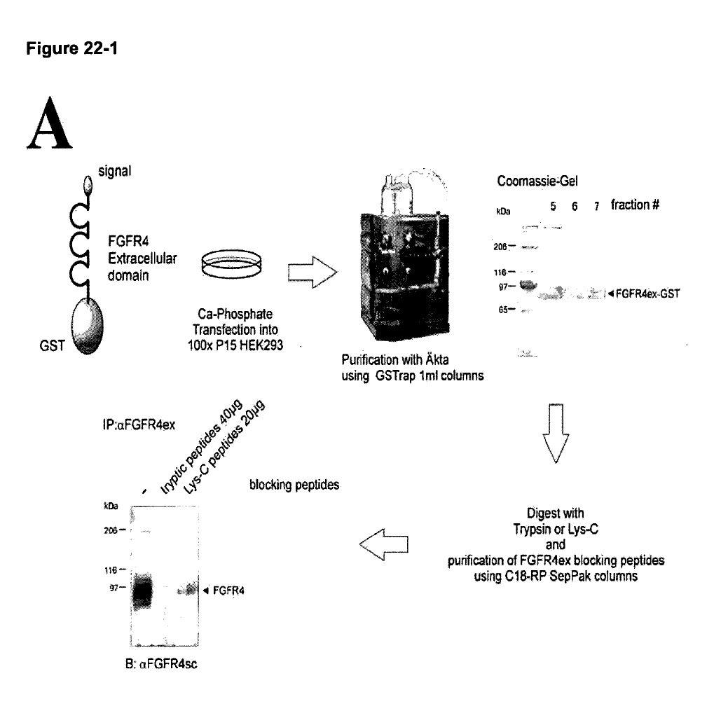

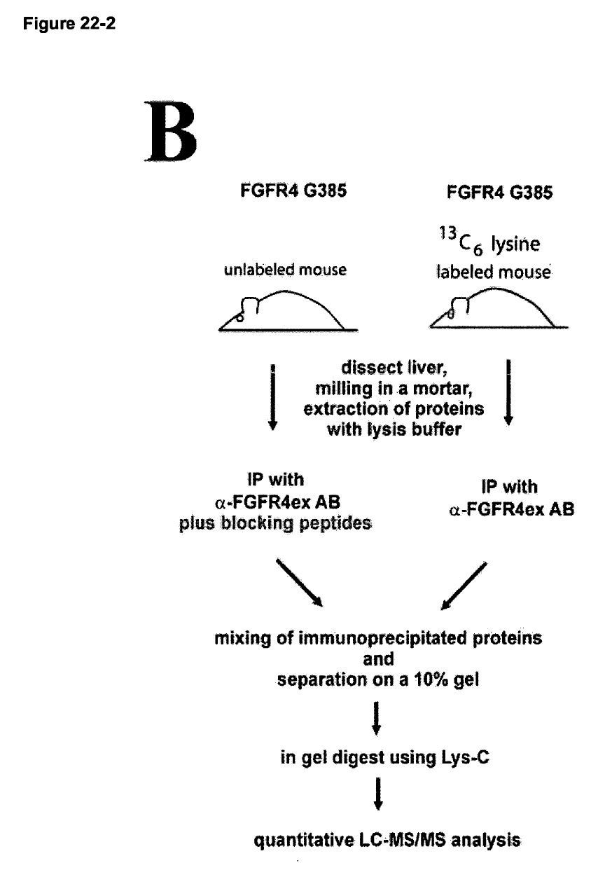

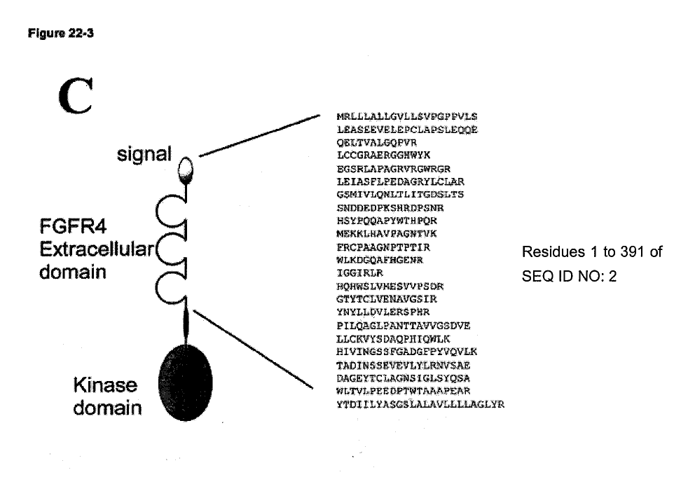

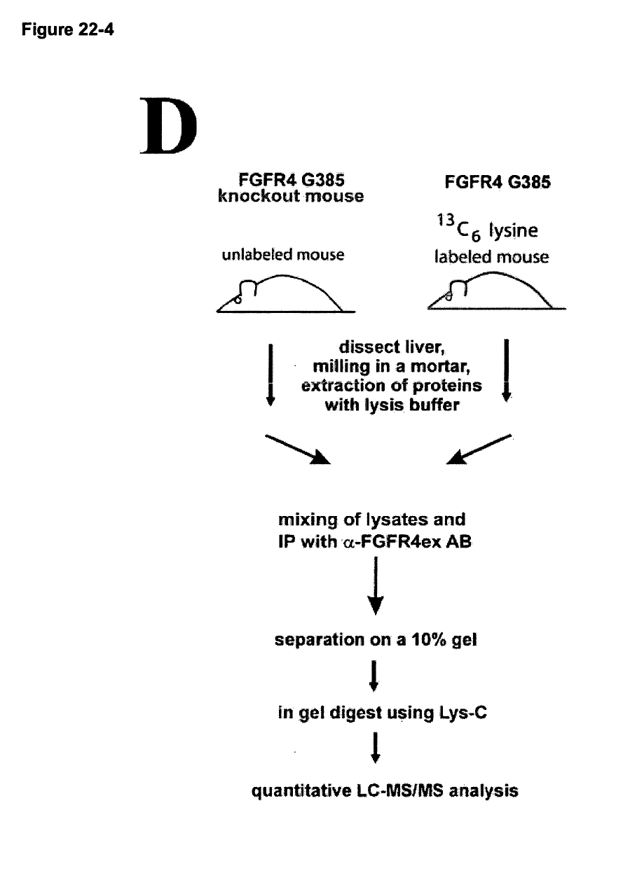

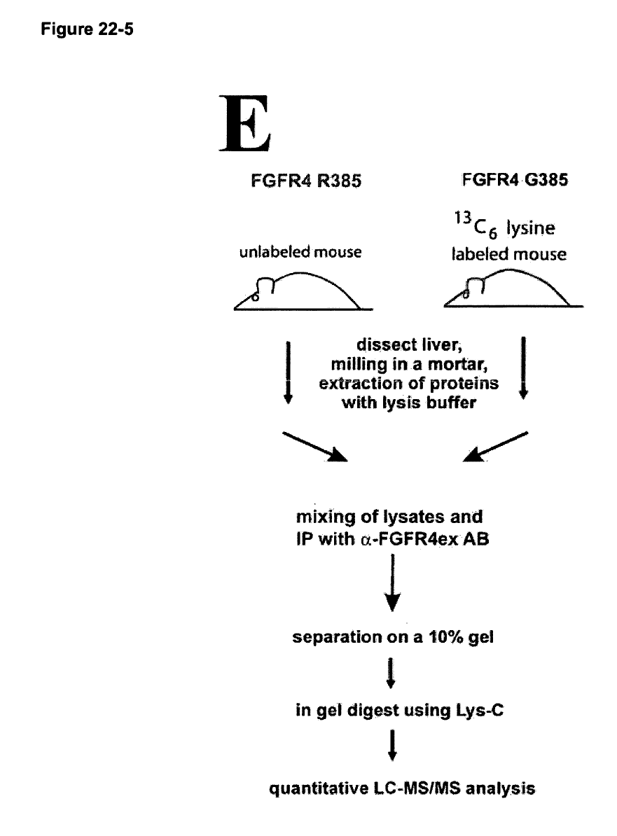

In FIG. 3 (A-D) every data point represents the values of one mouse. The values were normalised on body weight and plotted against the different investigated genotypes. The median of the values were plotted with asymmetric error bars.

(A/B) Sum of mass and size of investigated tumors. FGFR4 Arg385 carrying mice display a significantly increased tumor mass (Gly/Arg-p=0.03; Arg/Arg-p=0.002) and tumor size (Gly/Arg-p=0.01; Arg/Arg-p=0.0006) comparing to FGFR4 Gly385. Hence FGFR4 Arg385 promotes tumor growth.

(C/D) Percentage of mass and size of the occurring tumors compared to the whole mammary gland. FGFR4 Arg385 carrying mice display a significantly increased percentage of tumor mass (Gly/Arg-p=0.05; Arg/Arg-p=0.005) and size (Gly/Arg-p=ns; Arg/Arg-p=0.002) comparing FGFR4 Gly385.

(E) Comparison of FGFR4 Arg/Arg 385 and Gly/Gly 385 mice. White arrows indicate tumors. FGFR Arg/Arg 385 mice show a visible increased tumor mass and number

(F) Western Blot Analysis of immunoprecipitated FGFR4; FGFR4 is over expressed in WAP-TGFα/EGFR derived tumors compared to non-tumorigenic mammary gland; FGFR4 Arg/Arg displays a higher phosphorylation rate than FGFR4 Gly/Arg or Gly/Gly mice indicating a accelerated activity of the FGFR4 Arg/Arg 385 in WAP-TGFα/EGFR derived tumors

(G) HE and α-FGFR4 staining of WAP-TGFα/EGFR derived hyperplasic mammary glands and tumors; no obvious pathohistological changes in tumors derived from WAP-TGFα/EGFR mice carrying different FGFR4 alleles were found 1) H and α-FGFR4 staining of WAP-TGFα/EGFR derived hyperplasic mammary glands FGFR4 Arg/Arg is over expressed in hyperplasic mammary glands compared to FGFR4 Gly/Gly expression 2) 1) H and α-FGFR4 staining of WAP-TGFα/EGFR derived tumors; FGFR4 is over expressed and displays no differences between the different alleles

FIG. 4: FGFR4 Arg385 promotes Tumor Progression in the WAP-TGFα /EGFR

Mouse Mammary Tumor Model over time

In FIG. 4 (B-F) every time point indicates the value-median of at least three analysed mice. For every genotype these medians were plotted against time.

A) Time point of visible tumor incidence in FGFR4^{Gly385} and FGFR4^{Arg385}; Arg385 carrying mice display a significantly earlier tumor incidence (p=0.001); the median of the values were plotted with asymmetric error bars. Hence the FGFR4 Arg385 prematures visible tumor incidence

(B-D) FGFR4 Arg385 carrying mice establish a higher number, mass and size of tumors and show a faster progression over time.

(E-F) FGFR4 Arg385 carrying mice establish a higher percentage of tumor mass and size and show a faster progression over time

FIG. 5: FGFR4 Arg385 promotes Cancer Cell Metastasis in the WAP-TGFα/EGFR Mouse Mammary Tumor Model
(A) Time point of cancer cell metastasis incidence of investigated lungs; Arg385 carrying mice show a earlier occurrence of metastasis (p=ns)
(B) Analysis of occurred metastases; for every genotype size is plotted against number of metastases; FGFR4 Arg/Arg shows a accelerated number of metastases in every calculated size
(C) Analysis of occurred metastases; for every genotype size is plotted against number of metastases; FGFR4 Arg/Arg shows a accelerated number of metastases in every calculated size

FIG. 6: FGFR4 Arg385 promotes Cellular Transformation and facilitates cellular Survival in Mouse Embryo Fibroblasts (MEFs)
(A) Focus Formation Assay in MEFs; FGFR4 Arg/Arg385 carrying MEFs demonstrate a higher number of foci in every used oncogene
(B) Focus Formation Assay in MEFs; Growing of Foci was determined at different time points; FGFR4 Arg/Arg385 MEFs show an earlier time point of transformation and a higher progression over time
(C) Apoptosis in MEFs; MEFs were treated with 0.5 μM doxorubicin (dox) for 48 h;

apoptosis was measured via FAGS Analysis; FGFR4 Arg/Arg385 MEFs display a significantly (p=0.03) reduced number of apoptotic cells compared to FGFR Gly/Gly 385 cells; Similarly, Arg385 carrying MEFs display a significantly (p=0.03) reduced number of apoptotic cells compared to FGFR Gly/Gly 385 cells after 48 hrs of cisplatin treatment. Contrarily, treatment with taxol, a microtubules-interacting drug, causes no differences in apoptotic response after 48 hrs in FGFR4 Gly/Gly 385 MEFs compared to Arg/Arg385 MEFs. Hence FGFR4 Arg/Arg385 seems to facilitate cellular survival in response to DNA damaging drugs

FIG. 7: FGFR4 Arg385 and Gly385 Mice display identical Mammary Gland-Metrics

In FIG. 7 (A/B) every data point represents the value of one mouse plotted against the different investigated genotypes. The median of the values were plotted with asymmetric error bars.
(A) FGFR4 Gly/Gly385, Gly/Arg385 and Arg/Arg385 mice display an identical mass of mammary glands
(B) FGFR4 Gly/Gly385 Gly/Arg385 and Arg/Arg385 mice display an identical size of mammary glands

FIG. 8: FGFR4 Arg385 decreases the Time Point of Tumor Incidence in the WAP-TGFα/EGFR Model in the FVB Background

In FIG. 8A every data point represents the value of one mouse plotted against the different investigated genotypes. The median of the values were plotted with asymmetric error bars.

(A) Tumor onset in FGFR4^{Gly} and FGFR4^{Arg} mice transgenic for WAP-TGFα/EGFR; Arg385 carrying mice transgenic for WAP-TGFα/EGFR display a decreased time point of tumor onset in the FVB background

FIG. 9: Normal Proliferation, Life Span and Migration in FGFR4 Arg385 MEFs
(A) FGFR4 Arg385 shows no altered activity in MEFs; FGFR4 was immunoprecipitated and the amount of active receptor was analysed by a p-Tyr antibody; there was no differences detectable between the different FGFR4 alleles;
(B) FGFR4 Arg385 MEFs display no altered proliferation or a prolonged life span; MEFs were subcultured till senescence occurred, population doubling rate were calculated and plotted against time; 2) Apparently senescence MEFs were stained for β-galactosidase expression and the amount of senescent cells were calculated microscopically; there was no differences detectable between the different FGFR4 alleles;
(C) FGFR4 Arg385 MEFs display no altered migration; MEFs were analysed in the Boyden Chamber for migration for 16 h; there was no differences detectable between the different FGFR4 alleles

FIG. 10: The impact of FGFR4Arg385 on mammary gland metrics in absence of an oncogenic background.
(A) Analysis of mammary gland mass in FGFR4Gly/Gly385 (n=12), Gly/Arg385 (n=17) and Arg/Arg385 (n=12). Mice carrying the FGFR4Arg385 allele display no difference in the mass of mammary glands compared to mice homozygous for the FGFR4Gly385 allele;
(B) Analysis of mammary gland size in FGFR4Gly/Gly385 (n=12), Gly/Arg385 (n=16) and Arg/Arg385 (n=13) mice. Mice carrying the FGFR4Arg385 allele display no difference in the size of mammary glands compared to mice homozygous for the FGFR4Gly385 allele;

All data are shown as mean±SDM.

FIG. 11: The FGFR4Arg385 does not promote tumor progression in the MMTV-PymT mouse mammary tumor model, but decreases the time point of tumor incidence in the FVB background in mice transgenic for WAP-TGFα.
(A) Analysis of tumor size in 3 month old FGFR4Gly/Gly385 (n=8), Gly/Arg385 (n=13) and Arg/Arg385 (n=11) mice transgenic for MMTV-PyMT: Mice carrying the FGFR4Arg385 allele display no difference in the size of tumors compared to mice homozygous for the Gly385 allele;
(B) Analysis of tumor mass in 3 month old FGFR4Gly/Gly385 (n=8), Gly/Arg385 (n=13) and Arg/Arg385 (n=11) mice transgenic for MMTV-PyMT: Mice carrying the FGFR4Arg385 allele display no difference in the mass of tumors compared to mice homozygous for the FGFR4Gly385 allele;

All data are shown as mean±SDM, all p-values were calculated using the students T-test and values ≦0.03 were considered statistically significant.

FIG. 12: The FGFR4Arg385 is hyperactivated and promotes a more aggressive phenotype in the expression pattern of WAP-TGFα derived tumors.

Expression analysis of tumors derived from FGFR4Gly/Gly385 (n=10) or Arg/Arg385 (n=10) mice transgenic for WAP-TGFα after 6 month of tumor progression: target gene expression was analyzed via RT-PCR; GAPDH served as expression normalization value; expression values of FGFR4Arg/Arg385 tumors are blotted relatively to the expression values of Gly/Gly385 tumors and grouped regarding their physiological function; Tumors significantly overexpress genes involved in migration, invasion and vascularization in the presence of the FGFR4Arg385 allele; p21 is significantly downregulated in the presence of the

FGFR4Arg385 allele (MMP14-p=0.02, MMP13-p=0.021, MMP9-p=0.019, flk-1-p=0.02, CD44-p=0.02, CDK1-p=0.0091, p21-p=0.03);

All data are shown as mean±SDM; all p-values were calculated using the students T-test and values ≦0.03 were considered statistically significant.

FIG. 13: Expression Analysis and cell proliferation in MEFs transformed with EGFR or v-src.
(A) Western blot analysis of transformed FGFR4Gly/Gly385 (n=3) and Arg/Arg385 (n=3) MEFs: EGFR and v-src are not upregulated in control MEFs infected with empty pLXSN; v-src is overexpressed in MEFs infected with pLXSN-vsrc; EGFR is overexpressed in MEFs infected with pLXSN-EGFR; actin served as a loading control and normalization value for quantification; FGFR4Arg385 expression and activation is upregulated in MEFs transformed with EGFR.
(B) Proliferation Assay of transformed FGFR4Gly/Gly385 (n=3) and Arg/Arg385 (n=3) MEFs: cell number of seeded MEFs was monitored over time to calculate the population doubling rate; the presence of the FGFR4Arg385 allele does not influence the proliferation neither in control MEFs (empty pLXSN) nor in MEFs transformed with v-src or EGFR;

All data are shown as mean±SDM.

FIG. 14: The FGFR4Arg385 facilitates cellular transformation, migration, anchorage independent growth and branching in MEFs transformed with EGFR.
(A) Migration assay of stably EGFR transformed FGFR4Gly/Gly385 (n=3), and Arg/Arg385 (n=3) MEFs: Migratory capacity was analyzed microscopically after crystal violet staining (20×) and quantified via ELISA analysis. FGFR4Arg385 MEFs transformed with EGFR display a significantly (p=0.0005) increased migratory capacity;
(B) Soft Agar Colony Formation Assay of stably EGFR transformed FGFR4Gly/Gly385 (n=3), and Arg/Arg385 (n=3) MEFs: Anchorage independent growth was analyzed and quantified microscopically (20×) at the indicated time points. FGFR4Arg385 MEFs transformed with EGFR display a significantly increased capacity of anchorage independent growth in Soft Agar after 24-96 hours compared to FGFR4Gly385 MEFs (24 h-p=0.00004; 96 h-p=0.00003);
(C) Invasion Assay in Matrigel of stably EGFR transformed FGFR4Gly/Gly385 (n=3) and Arg/Arg385 (n=3) MEFs: branching in Matrigel was analyzed and quantified microscopically (20×) at the indicated time points; FGFR4Arg385 MEFs transformed with EGFR display a significantly increased invasion in Matrigel after 96 hours compared to FGFR4Gly385 MEFs (p=0.00009); All data are shown as mean±SDM; all p-values were calculated using the students T-test and values ≦0.03 were considered statistically significant.

FIG. 15: FGFR4Arg385 does not promote migration, anchorage independent growth and branching in MEFs transformed with v-src or stably expressing the empty pLXSN vector.
(A) Migratory capacity of FGFR4Gly/Gly385 (n=3) or Arg/Arg385 (n=3) MEFs transformed with v-src or overexpressing the empty pLXSN-vector: MEFs display no difference in their migratory capacity regarding the FGFR4 alleles.
(B) Anchorage independent growth of FGFR4Gly/Gly385 (n=3) or Arg/Arg385 (n=3) MEFs transformed with v-src or overexpressing the empty pLXSN-vector: MEFs transformed with v-src display no difference in anchorage independent growth regarding the FGFR4 alleles. MEFs stably expressing the empty pLXSN-vector are not able to grow anchorage independent.
(C) Matrigel outgrowth of FGFR4Gly/Gly385 (n=3) or Arg/Arg385 (n=3) MEFs transformed with v-src or overexpressing the empty pLXSN-vector: MEFs transformed with v-src display no difference in Matrigel outgrowth regarding the FGFR4 alleles. MEFs stably expressing the empty pLXSN-vector are not able to branch in Matrigel.

FIG. 16: Simplified scheme of the experimental setup to analyze FGFR4 interaction partners in MDA-MB-231 cells expressing either empty pLXSN vector, pLXSN-FGFR4 Gly388 or pLXSN-FGFR Arg388; cell lines were subcultured in media containing modified amino acids for SILAC labelling; between MDA-MB-231 cells expressing FGFR4 Gly388 and Arg388 a lable switch was performed to verify the results. After cell lysis, lysates were pooled 1:1; FGFR4 and its interactors were immunoprecipitated and subjected for in-gel digest with Trypsin and LysC followed by quantitative LC-MS/MS analysis.

FIG. 17: Validation of the EGFR/FGFR4 interaction; A) Co-Immunoprecipitation of EGFR and FGFR4 in MDA-MB-231 cells overexpressing the empty pLXSN, pLXSN-Gly388 and -Arg388: Interaction of EGFR and FGFR4 Arg388 seems to be stronger than EGFR and FGFR4 Gly388; B) EGFR-FGFR4 interaction upon EGF-stimulation: increased phosphorylation of the EGFR and accelerated FGFR4 interaction and activation in MDA-MB-231 cells expressing the FGFR4 Arg388; C)

Quantification of Western Blot Analysis of EGF stimulated MDA-MB-231 cells: MDA-MB-231 cells expressing the FGFR4 Ag388 display an accelerated EGFR and Akt activation, total EGFR and tubulin served as normalization value for quantification, respectively; the co-immunoprecipitated FGFR4 Arg388 displays a accelerated binding to the EGFR and increased activation comparetd to the co-immunoprecipitated FGFR4 Gly388.

FIG. 18: Western Blot analysis of MEFs derived from FGFR4 Gly385 or Arg385 homozygous mice transformed with EGFR upon EGF and TGFα stimulation; A) MEFs transformed with EGFR display an increased and prolonged activation of Akt upon EGF and TGFα stimulation when expressing the FGFR4 Arg385 allele; B) MEFs transformed with EGFR display an significantly increased activation of the EGFR upon EGF and TGFα stimulation when expressing the FGFR4 Arg385 allele (EGF5′-p=0.000073, EGF10′-p=0.0025, TGFα5′-p=0.07, TGFα10′-p=0.01); actin served as a normalization value for quantification C) In MEFs, transformed with EGFR, FGFR4 gets activated upon EGF and TGFα stimulation whereas the FGFR4 Arg385 displays an increased phosphorylation compared to the FGFR4 Gly385; All data are shown as mean±SDM; all p-values were calculated using the students T-test and values ≦0.03 were considered statistically significant.

FIG. 19: Biological properties of MDA-MB-231 cells expressing empty pLXSN, pLXSN-Gly388 or pLXSN-Arg388; A) MDA-MB-231 cells do not alter their proliferative capacity by overexpressing the FGFR4; B) MDA-MB-231 cells display a partly significantly increased migratory capacity by overexpressing the FGFR4 (FGFR4 Arg388-p=0.001); MDA-MB-231 cells overexpressing the FGFR4 Arg388 allele display a significantly accelerated migration compared to MDA-MB-231 cells expressing the FGFR4 Gly388 allele (FGFR4 Arg388-p=0.001); All data are shown as mean±SDM; all p-values were calculated using the students T-test and values ≦0.03 were considered statistically significant.

FIG. 20: Impact of Gefitinib in MDA-MB-231 cells expressing empty pLXSN, pLXSN-Gly388 or pLXSN-Arg388 on proliferation, apoptosis and migration; A) MDA-MB-231 cells overexpressing the FGFR4 Arg388 allele display a increased sensitivity (IC₅₀=9.53) towards Gefitinib compared to FGFR4 Gly388 or control cells (IC₅₀=18.72); B) MDA-MB-231 cells display a significant increase in apoptosis in the presence of the FGFR4 Arg388 allele compared to the FGFR4 Gly388 towards Gefitinib (20 μM-p=0.012;10 μM-p=0.0022); C) MDA-MB-231 cells display a decrease in migration in the presence of the FGFR4 Arg388 allele compared to the FGFR4 Gly388 in response to Gefitinib; All data are shown as mean±SDM; all p-values were calculated using the students T-test and values ≦0.03 were considered statistically significant.

FIG. 21: In Vivo labelling of C57BL/6 mice: mice were fed with a diet containing either the natural or ¹³C₆-substituted version of lysine; The efficiency of labeling is dependent on the cell proliferation rate of the specific tissue; the F2 generation is labeled completely (Kruger et al., 2008).

FIG. 22: Investigation of hepatic interaction partners of the FGFR4 via in vivo SILAC: A) Synthesis of blocking peptides; HEK293 were used to transiently transfect a vector containing the extracellular domain of the FGFR4 tagged with GST. Via specific signal petides, the recombinant protein can be delivered to the cell media; after digestion with either Trypsin or Lysin the efficiency of the blocking peptides were tested in an immunoprecipitation experiment with FGFR4.

B) Experimental scheme to analyze interaction partners of hepatic FGFR4 via blocking peptides; to enable a quantifiable analyis, the labelled SILAC mouse was used as an internal standard; livers of labelled and unlabelled mice were dissected and lysed; with unlabelled liver-lysates FGFR4 was immunoprecipitated in the presence of the blocking peptides preventing the binding of FGFR4 with the antibody for the detection of unspecific binding partners; in labelled liver-lysates, FGFR4 was immunoprecipitated without blocking peptides to analyze FGFR4 binding partners.

C) Sequence analysis for the generation of specific blocking peptides.

D) Experimental scheme to analyze interaction partners of hepatic FGFR4 via FGFR4 KO mice; to enable a quantifiable analyis, the labelled SILAC mouse was used as an internal standard; livers of labelled and unlabelled mice were dissected, lysed and mixed together for FGFR4 immunoprecipitation.

E) Experimental scheme to analyze interaction partners of hepatic FGFR4 Arg385; to enable a quantifiable analyis, the labelled SILAC mouse was used as an internal standard; livers of labelled and unlabelled mice were dissected, lysed and mixed together for FGFR4 immunoprecipitation.

### DETAILED DESCRIPTION OF THE INVENTION

The present invention provides a non-human animal, preferably a rodent animal comprising an endogenous gene encoding a modified FGFR4 protein, wherein the modification is an amino acid substitution in the wild-type FGFR4 of said non-human rodent at the amino acid position corresponding to amino acid position 385 of SEQ ID NO: 1, i.e., mouse FGFR. Alternatively, the modification may be a deletion of at least the amino acid at position 385 of SEQ ID NO: 1 or any corresponding sequence or an insertion of at least one amino acid at position 385 of SEQ ID NO: 1 or any corresponding sequence.

In one embodiment, the invention provides a rodent which is a mouse or a rat comprising an endogenous gene encoding a modified FGFR4 protein, wherein the modification is an amino acid substitution in the wild-type FGFR4 of said mouse or rat wherein in case of said mouse the amino acid substitution is at the amino acid position 385 of SEQ ID NO: 1 or wherein in case of said rat the amino acid substitution is at the amino acid position 386 of SEQ ID NO: 4.

In one embodiment, the invention provides a rodent which is a mouse comprising an endogenous gene encoding a modified FGFR4 protein, wherein the modification is an amino acid substitution in the wild-type FGFR4 of said mouse wherein the amino acid substitution is at the amino acid position 385 of SEQ ID NO: 1.

The term “corresponding to” as defined herein refers to the amino acid position of FGFR4 orthologues, isoforms, mature forms, or variants as described herein that defines the position 385 of SEQ ID NO: 1 in those sequences. It is obvious to the skilled person that in this context the gene of the modified FGFR4 reflects the modification in the mouse FGFR4 protein according to SEQ ID NO: 1 or any other of the herein-mentioned sequences on the amino acid level. Where the sequences of the FGFR4 gene flanking the modification do not encode amino acids identical to those at the corresponding positions in the amino acid sequences of the mouse FGFR4 protein defined above, the skilled artisan will be readily able to align the amino acid sequences encoded by the flanking sequences with the corresponding amino acids of the mouse FGFR4 protein, preferably by using the below-mentioned method of determining amino acid sequence identity, and determine whether a modification in the mouse FGFR4 protein of the kind mentioned above is reflected by the amino acid sequence encoded by said gene. In case of an amino acid substitution or insertion, the modification is preferably reflected by the amino acid sequence encoded by the gene in such a way that an identical amino acid or amino acid sequence is found at the corresponding position of the protein encoded by the allele. In case of an amino acid deletion, the modification is preferably reflected by the amino acid sequence encoded by the gene in such a way that an identical or corresponding amino acid or amino acid sequence is deleted at the corresponding position of the protein encoded by the gene.

For example, the protein mentioned above may be, for example, a mouse wild-type FGFR4 protein, e.g., with the sequence as disclosed in SEQ ID NO: 1. The modification, e.g., the amino acid substitution, then affects the amino acid position 385 of SEQ ID NO: 1 which is a glycine. Alternatively, the modified FGFR4 protein may be any orthologue of the mouse FGFR4 protein protein according to SEQ ID NO: 1 with respect to the animal, e.g. from from a vertebrate, preferably from a mammal, and more preferably from a rodent, e.g., Mus (e.g., mice) or Rattus (e.g. rat), or from Oryctologus (e.g. Rabbit) or Mesocricetus (e.g., hamster). In this case, the amino acid substitution may affect the amino acid position that corresponds to the amino acid position 385 in SEQ ID NO: 1. For example, in the rat sequence according to, e.g., SEQ ID NO: 4, the amino acid position 385 of the mouse sequence corresponds to amino acid position 386.

In one embodiment, in said rodent, e.g. mouse or rat, the animal according to anyone of claim 1 or 4 wherein in said rodent the amino acid position corresponding to amino acid position 385 of SEQ ID NO: 1 is glycine.

In another embodiment, in said rodent, e.g. mouse or rat, the amino acid substitution is with an amino acid different from glycine.

The modified FGFR4 protein of the non-human animals, e.g. a rodent, as described herein may also be a variant of the mouse or rat FGFR4 protein according to SEQ ID NO: 1 and SEQ ID NO: 4, or of said orthologue, allelic variant or otherwise, wherein certain amino acids or partial amino acid sequences have been replaced, added, or deleted.

Preferably, the amino acid position 385 of the wild type FGFR4 sequence according to SEQ ID NO: 1 or the corresponding amino acid position in the non-human animals, e.g., a rodent, is replaced by an amino acid different from glycine, e.g., an amino acid with different size and/or polarity, i.e., a non-conservative amino acid substitution. Non conservative substitutions are defined as exchanges of an amino acid by another amino acid listed in a different group of the five standard amino acid groups shown below:
1. small aliphatic, nonpolar or slightly polar residues: Ala, Val, Ser, Thr,(Pro), (Gly);
2. negatively charged residues and their amides: Asn, Asp, Glu, Gln;
3. positively charged residues: His, Arg, Lys;
4. large aliphatic, nonpolar residues: Met, Leu, Ile, Val, (Cys);
5. large aromatic residues: Phe, Trp.

Conservative substitutions are defined as exchanges of an amino acid by another amino acid listed within the same group of the five standard amino acid groups shown above. Three residues are parenthesized because of their special role in protein architecture. Gly is the only residue without a side-chain and therefore imparts flexibility to the chain. Pro has an unusual geometry which tightly constrains the chain. Cys can participate in disulfide bonds.

In one embodiment of the invention, the glycine residue at position 385 of the FGFR4 according to SEQ ID NO: 1 or the corresponding amino acid position in the non-human animal, e.g. a rodent, is replaced by another residue than glycine, preferably by another residue than Ala, Val, Ser, Thr, (Pro) and preferably with an amino acid with a charged side chain, i.e., with positively charged side chain such as a lysine, arginine or histidine, and more preferably arginine.

In a preferred embodiment the non-human animal, e.g., rodent, expresses the amino acid sequences shown in SEQ ID NO: 5 or SEQ ID NO: 6.

The non-human animal of the present invention, e.g. rodent, is not limited to comprise the modification of the glycine residue at position 385 of the FGFR4 according to SEQ ID NO: 1 or SEQ ID NO: 4 or at the corresponding position in other FGFR4 orthologues. Rather the term “modification” of the FGFR4 of the non-human animals, e.g., rodent, as described herein encompasses any modification in the FGFR4 as long as they do result in the phenotype as described herein, e.g., amino acid substitutions, deletions or insertions. Insertional amino acid sequence modifications are those in which one or more amino acid residues are introduced into a predetermined site in the protein although random insertion is also possible with suitable screening of the product. Deletional modifications are characterized by the removal of one or more amino acids from the sequence leading, e.g., to a frame-shift or insertion of a stop codon. Substitutional amino acid modifications are those in which at least one residue in the sequence has been removed and a different residue inserted in its place.

Accordingly, a further aspect of the invention is a non-human animal, e.g., rodent, comprising an endogenous gene encoding a modified FGFR4 protein, wherein the modification is an amino acid modification as described above, e.g., an amino acid substitution at at least one amino acid position compared to the wild-type FGFR4 protein, preferably an FGFR4 protein according to SEQ ID NO: 1, which modification, if present in at least some or all or essentially all cells of said animal in a heterozygous or homozygous manner, results in a phenotype associated with an alteration in tumor progression and/or formation as described herein further below, e.g. an increased rate of tumor growth and/or metastasis formation compared to the wild-type animal. In a preferred embodiment said animal additionally expresses in the genome of at least some of its cells a transgene encoding a TGF-α protein, e.g., in mammary cells by expression under the control of an appropriate promotor, e.g., the WAP-promotor or in liver cells, e.g. hepatocyctes, by the expression under the control of an appropriate promotor, e.g. the albumin promotor, α-1 antitrypsin promotor, or TGF-α metallothionein 1 promotor.

Preferably, the modified FGFR4 in the non-human animal of the invention, e.g., rodent, is at least 70%, 75%, 80%, 85%, 90%, 95%, 98%, or 99% identical with the wild-type FGFR4 sequence of the animal, preferably, the vertebrate, more preferably the mammal and most preferably the rodent, e.g., mouse or rat FGFR4 according to SEQ ID NO: 1 or SEQ ID NO: 4. In one embodiment the modified FGFR4 protein is identical to its wild-type protein except for the amino acid substitution corresponding to amino acid position 385 of the FGFR4 according to SEQ ID NO: 1.

The following definitions apply to any reference to nucleic acid or amino acid sequence identity throughout the present specification:

The term “sequence identity” refers to the degree to which two polynucleotide, protein or polypeptide sequences are identical on a residue-by-residue basis over a particular region of comparison.

The phrases “percent amino acid identity” or “% amino acid identity” refer to the percentage of sequence identity found in a comparison of two or more amino acid or nucleic acid sequences. Percent identity can be readily determined electronically, e.g., by using the MEGALIGN program (DNASTAR, Inc., Madison Wis.). The MEGALIGN program can create alignments between two or more sequences according to different methods, one of them being the clustal method. See, e.g., Higgins and Sharp (Higgins and Sharp 1988). The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity.

Percent identity can also be readly determined electronically, by using the MultAlin software (Carpet 1988).

Another method of determining amino acid identity between two protein sequences for the purposes of the present invention is using the “Blast 2 sequences” (bl2seq) algorithm described by Tatusova et al. (Tatusova and Madden 1999). This method produces an alignment of two given sequences using the “BLAST” engine. On-line access of “blasting two Sequences” can be gained via the NCBI server at http://www.ncbi.nlm.nih.gov/blast/bl2seq/bl2.html. The stand-alone executable for blasting two sequences (bl2seq) can be retrieved from the NCBI ftp site (ftp://ftp.ncbi.nih.gov/blast/executables). Preferably, the settings of the program blastp used to determine the number and percentage of identical or similar amino acids between two proteins are the following:

Program: blastp

Matrix: BLOSUM62

Open gap penalty: 11

Extension gap penalty: 1

Gapxdropoff: 50

Expect: 10.0

Word size: 3

Low-complexity filter: on

The comparison of two or more amino acid or nucleic acid sequences to determine sequence identity can be performed between orthologue sequences, preferably between mouse and rat sequences.

Preferably, the wild type residue of the modified FGFR4 protein wherein the modification is at least one amino acid substitution compared to the wild-type FGFR4 protein, preferably an FGFR4 protein according to SEQ ID NO: 1, which modification, if present in at least some or all or essentially all cells of an animal as described herein, e.g., a rodent, in a heterozygous or homozygous manner, results in a phenotype associated with an alteration in tumor progression and/or formation, e.g. an increased rate of tumor growth and/or metastasis formation compared to a wild-type animal is replaced by an amino acid with different size and/or polarity, i.e., a non-conservative amino acid substitution, as defined above. In a preferred embodiment said animal additionally expresses in the genome of at least some of its cells a transgene encoding a TGF-α protein or any other gene inducing breast cancer. Alternatively, said animal additionally expresses in the genome of at least some of its cells a gene inducing hepatocellular cancer, e.g. p53 or c-myc.

The term “phenotype” as used herein refers to one or more morphological, physiological, behavioral and/or biochemical traits possessed by a cell or organism that result from the interaction of the genotype and the environment. Thus, the non-human animal of the present invention, e.g. rodent, displays one or more readily observable abnormalities compared to the wild type animal. In a preferred embodiment the animal of the invention shows at least 1, at least 2, at least 3, or at least 4 abnormal phenotypical features selected from any of the above categories.

The term “phenotype associated with an alteration in tumor progression” as referred to throughout the present application may be characterized by an increased rate of tumor growth and/or metastasis formation compared to a wild-type animal. Further characterization that falls under the definition of the this phenotype may be found below in the Examples. A preferred tumor in this respect is a mammary tumor or a liver (hepatocellular) tumor.

The endogenous promotor of the FGFR4 gene or transgene as described above in connection with the non-human animals, e.g. rodent, may be replaced by a heterologous promotor, e.g., a promotor imposing a different tissue specificity of expression upon the gene e.g., the WAP-promotor for mammary cells, villin-promotor for colorectal cells, or the albumin promotor α-1 antitrypsin promotor, or TGF-α metallothionein 1 promotor for hepatocytes or a temporally controlled promotor, e.g., a promotor that is inducible by chemical or physical means, e.g., the tet-CRE system.

The term “modified” or “modification” as used herein refers to an alteration compared to the wild type. The term “mutant” or “modified” as used herein in connection with the FGFR4 protein sequences and nucleic acid sequences relating thereto refers to an alteration in the sequence compared to the corresponding wild type FGFR4.

The non-human animals as described herein may be a vertebrate animal, preferably a mammal. In a further preferred embodiment, the non-human animal is a rodent.

In particular, the rodent may be selected from the genus Mus (e.g., mice), Rattus (e.g. rat), Oryctologus (e.g. Rabbit) or Mesocricetus (e.g., hamster). A particular preferred non-human animal is a mouse or a rat.

The non-human animal models of the invention as described herein, e.g. a rodent, expresses the endogenous modified FGFR4 protein or gene as described herein or the transgene as described herein in at least some of its cells, e.g., as mosaic animal, such as chimeric animals, or in case the modified FGFR4 protein is expressed by a gene with a heterologous promotor as defined above. The non-human animal model of the invention, e.g. a rodent, may also express the endogenous modified FGFR4 protein as described herein in all of its cells, e.g., by expressing the FGFR4 protein from a nucleic acid encoding said FGFR4 under the control of an ubiquitarily expressed promotor. The modified FGFR4 protein may also be translated from a nucleic acid encoding the nucleic acid encoding said FGFR4 protein under the control of the endogenous FGFR4 promotor. The cells of the non-human animal as described, e.g. rodent, are at least heterozygous with respect to the amino acid modification, e.g., substitution, as described herein. Alternatively, the cells may also be homozygous.

The invention furthermore encompasses non-human animals, e g. a rodent, comprising mature modified FGFR4 proteins, or their vertebrate orthologues being modified as described herein, e.g., the specific orthologues referred to above, which comprise an amino acid or amino acid sequences corresponding to the FGFR4 proteins as defined herein. As used herein, a “mature” form of a polypeptide or protein may arise from a posttranslational modification. Such additional processes include, by way of non-limiting example, proteolytic cleavage, e.g., cleavage of a leader sequence, glycosylation, myristoylation or phosphorylation. In general, a mature polypeptide or protein according to the present invention may result from the operation of one of these processes, or a combination of any of them.

The nucleic acid or gene encoding the amino acid substitution of the invention may be present in germ cells or somatic cells of the non-human vertebrate animal, or both.

The non-human animals as described herein, e.g. a rodent, may in addition to the modification of the FGFR4 as described herein displaying uncontrolled cell growth, preferably cancer and/or metastasis formation.

The term “uncontrolled cell growth” as used in the present invention relates to any state characterized by uncontrolled growth, e.g., cancer. Examples of cancer are breast cancer, lung cancer, colorectal cancer, hepatocellular cancer, prostate cancer, melanoma, and/or pancreatic cancer. The term “uncontrolled cell growth” also comprises uncontrolled division of cells, i.e., growth and/or division beyond the growth and/or division of the same cells in a non-uncontrolled cell growth state. Techniques how to determine uncontrolled cell growth are known by the person skilled in the art, e.g., visual inspection of the cells (histology).

Uncontrolled cell growth may be triggered by any method or treatment that is known by the skilled person to lead to uncontrolled growth and/or division of cells, i.e., irradiation, e.g., with UV-light, or treatment with a cancer-inducing agent, e.g., dimethylhydrazine (DMH), azoxymethane (AOM), N-methyl-N-nitro-N-nitrosoguanidine (MNNG), N-methyl-N-nitrosourea (MNU), ethyl-nitroso-urea (ENU) or 12-0-tetradecanoylphorbol-13-acetate (TPA). Alternatively, it may be triggered by the expression of a transgene comprising an oncogene, i.e., a gene which is deregulated and which deregulation participates in the onset and development of cancer. Examples of such genes are TGF-α, TGF-β, HGF, IGF-I, PyV-mz, erb-B2, RET, Cyclin D1, EGFR, v-src, c-kit, HER2, Trp53, INK4a/ARF, E2F-1, Cyclin A, myc, p53, ras, Rb, particularly TGF-α, TGF-β, EGFR, v-src, c-kit, HER2, erb-B2, p53, myc, or ras and more particularly TGF-α (SEQ ID NO: 74) or/and EGFR (SEQ ID NO: 76). The transgene may be expressed in the whole organism or in individual cells or tissues, e.g., in mammary cells, lung cells, colorectal cells, hepatocytes, prostate cells, skin cells, or pancreatic cells, particularly β-islets. As described above, expression of the transgene in all or at least some cells may be achieved by the use of appropriate promotors.

In a further aspect of the invention, the non-human animal, e.g. rodent, comprising an endogenous gene encoding a modified FGFR4 protein, wherein the modification is, e.g., an amino acid substitution in the wild-type FGFR4 of said non-human animal at the amino acid position corresponding to amino acid position 385 of SEQ ID NO: 1 as described herein develop a phenotype associated with an alteration in tumor progression and/or formation as described herein.

In another aspect, the non-human animal, e.g. rodent, comprising an endogenous gene encoding a modified FGFR4 protein, wherein the modification is a modification as described herein, e.g., an amino acid substitution at at least one amino acid position compared to the wild-type FGFR4 protein, preferably an FGFR4 protein according to SEQ ID NO:1, which modification, if present in at least some or all or essentially all cells of said animal in a heterozygous or homozygous manner, results in a phenotype associated with an alteration in tumor progression and/or formation as described herein, e.g. an increased rate of tumor growth and/or metastasis formation compared to a wild-type animal develops a phenotype associated with an alteration in tumor progression and/or formation as described herein. In a preferred embodiment said animal additionally expresses in the genome of at least some of its cells a transgene encoding a TGF-α protein.

In a further aspect of the invention, the non-human animal, e.g. rodent, comprising an endogenous gene encoding a modified FGFR4 protein, wherein the modification is an amino acid substitution in the wild-type FGFR4 of said non-human animal, e.g. rodent, at the amino acid position corresponding to amino acid position 385 of SEQ ID NO: 1 as described herein displaying in addition to the modification of the FGFR4 as described herein uncontrolled cell growth as described above and/or metastasis formation and develops a phenotype associated with an alteration in tumor progression and/or formation as described herein.

In another aspect, the non-human animal, e.g. rodent, comprising an endogenous gene encoding a modified FGFR4 protein, wherein the modification is a modification as described herein, e.g., an amino acid substitution at at least one amino acid position compared to the wild-type FGFR4 protein, preferably an FGFR4 protein according to SEQ ID NO:1, which modification, if present in at least some or all or essentially all cells of said animal in a heterozygous or homozygous manner, results in a phenotype associated with an alteration in tumor progression as described herein, e.g. an increased rate of tumor growth and/or metastasis formation compared to a wild-type animal, displaying in addition to the modification of the FGFR4 as described herein uncontrolled cell growth as described above and/or metastasis formation and develops a phenotype associated with an alteration in tumor progression and/or formation as described herein. In a preferred embodiment, said animal additionally expresses in the genome of at least some of its cells a transgene encoding a TGF-α protein.

As will be apparent from the previous explanations, the non-human animals according to the invention, e.g. rodent, may be produced by any technique known to the person skilled in the art, e.g., by the application of procedures, which result in an animal with a genome that incorporates/integrates exogenous genetic material, e.g., in such a manner as to modify or disrupt the function of the normal FGFR4 gene or protein or in such a manner to express a modified FGFR4 as described above or in such a manner as to integrate additional copies of a gene, e.g., a transgene comprising an oncogene as described herein. These techniques may include but are not limited to micro-injection, electroporation, cell gun, cell fusion, nucleus transfer into anucleated cells, micro-injection into embryos of teratocarcinoma stem cells or functionally equivalent embryonic stem cells. One embodiment of a procedure for generating an animal of this invention is one according to “Material and Methods” further below.

In case of production of a transgenic animal with a transgene that comprises an oncogene as described above, the genetic material encoding the transgene may be micro-injected into the pro-nucleus of a fertilized ovum, a process that is known by the skilled person. The insertion of DNA is, however, a random process. The manipulated fertilized ovum is transferred into the oviduct of a recipient female, or foster mother that has been induced to act as a recipient by mating with a vasectomized male. The resulting offspring of the female is likewise tested to determine which animals carry the transgene.

The present invention further provides for inbred successive lines of animals carrying the nucleic acid encoding the modified FGFR4 protein of the present invention that offer the advantage of providing a virtually homogeneous genetic background. A genetically homogeneous line of animals provides a functionally reproducible model system for conditions or symptoms associated with uncontrolled cell growth, with alterations in tumor progression, and/or metastasis formation.

The animals of the invention can also be used as a source of primary cells, e.g., mouse embryonic feeder cells (MEF), from a variety of tissues, for cell culture experiment, including, but not limited to, the production of immortalized cell lines by any methods known in the art, such as retroviral transformation.

Such primary cells or immortalized cell lines derived from any one of the non-human vertebrate animals described and claimed herein are likewise within the scope of the present invention. In one embodiment, such primary cells, e.g. MEFs, are derived from an animal such as described herein which comprises in all of its cells the modified FGFR4 encoding gene as described herein. Such cells may be heterozygous or homozygous with respect to said modified FGFR4. In another embodiment, such primary cells, e.g. MEFs, additionally comprise a nucleic acid encoding EGFR (SEQ ID NO: 76 or SEQ ID NO: 77) or EGFR protein. The EGFR nucleic acid or the EGFR protein may be present transiently, e.g. via infection, or stably, e.g. as described in the Examples. Such immortalized cells from these animals may advantageously exhibit desirable properties of both normal and transformed cultured cells, i.e., they will be normal or nearly normal morphologically and physiologically, but can be cultured for long, and perhaps indefinite periods of time. The primary cells or cell lines derived thereof may furthermore be used for the construction of an animal model according to the present invention.

In other embodiments cell lines according to the present invention may be prepared by the insertion of a nucleic acid construct comprising the nucleic acid sequence of the invention or a fragment thereof comprising the codon imparting the above-described phenotype to the animal model of the invention. Suitable cells for the insertion include primary cells harvested from an animal as well as cells, which are members of an immortalized cell line. Recombinant nucleic acid constructs of the invention, described below, may be introduced into the cells by any method known in the art, including but not limited to, transfection, retroviral infection, micro-injection, electroporation, transduction or DEAE-dextran. Cells, which express the recombinant construct, may be identified by, for example, using a second recombinant nucleic acid construct comprising a reporter gene, which is used to produce selective expression. Cells that express the nucleic acid sequence of the invention or a fragment thereof may be identified indirectly by the detection of reporter gene expression.

It will be appreciated that the non-human animals of the invention, e.g. rodents, are useful in various respects in connection with phenotypes relating to an alteration in tumor progression and/or formation; with uncontrolled cell growth; with medical conditions associated with uncontrolled cell growth, e.g. cancer, tumor formation and/or progression; with metastasis formation; with uncontrolled cell growth, and/or uncontrolled cell division.

Accordingly, one aspect of the present invention is the use of the non-human animal, e.g. rodent, primary cells, or cell lines as described herein as a model for studying the molecular mechanisms of, or physiological processes associated with uncontrolled cell growth, such as cancer and/or metastasis formation, preferably in breast cancer, lung cancer, colorectal cancer, hepatocellular cancer, prostate cancer, melanoma, and/or pancreatic cancer. This may be done, e.g., by performing differential proteomics analysis, using techniques including , e.g., 2D-gel electrophoresis, protein chip microarrays, or mass spectrophotometry on tissue displaying uncontrolled cell growth, e.g., cancer, as described herein. This may also be done on nucleic acid level by, e.g., differential display or cDNA microarrays.

A further aspect of the invention is the use of the non-human animal, e.g. rodent, primary cells, or cell lines as described herein as a model for studying the identification and/or testing of an agent useful in the prevention, amelioration or treatment of uncontrolled cell growth, such as cancer and/or metastasis formation, preferably in breast cancer, lung cancer, colorectal cancer, hepatocellular cancer, prostate cancer, melanoma, and/or pancreatic cancer. The agent to be tested can be administered to an animals of the present invention, e.g., a rodent, and any technique known by the skilled person may be used in order to monitor the effect of the agent to be tested. The non-human animal, e.g. rodent, may be exposed to the agent to be tested at different stages of uncontrolled cell growth, e.g., cancer and, e.g., the mass, area and percentage of occurring tumors and/or metastasis formation; the percentage of mass and area of the tumor and/or metastasis formation compared to to the whole tissue in which the tumor or metastases occurrs; the expression profile of FGFR4 within the tumor compared to the same tissue without a tumor; the phosphorylation status of FGFR4 in tumor tissue compared to non-tumor tissue, or Focus Formation Assay may be determined (cf. also Examples below).

Also within the scope of the invention is the use of the non-human animal, e.g. rodent, primary cells, or cell lines as described herein as a model for studying the identification of a protein and/or nucleic diagnostic marker for uncontrolled cell growth, such as cancer and/or metastasis formation, preferably in breast cancer, lung cancer, colorectal cancer, hepatocellular cancer, prostate cancer, melanoma, and/or pancreatic cancer, such as diagnostic markers relating to genes or gene products that play a role in the early phase, the intermediate phase, and/or the late phase of medical conditions associated with an uncontrolled cell growth, such as cancer as described herein, or diagnostic markers for diseases associated with FGFR4 modifications as described herein.

It will be appreciated that such diagnostic markers may relate to the FGFR4 gene or its protein product. However, it will be understood that the non-human animal according to the present invention, e.g. a rodent, can also be used to identify markers relating to other genes or gene products that affect FGFR4 gene or protein expression or function, or the expression or function of which is affected by the FGFR4 protein. Moreover, since the non-human animal of the invention, e.g. a rodent, represents a highly useful model system for studying the pathogenesis of medical conditions associated with uncontrolled cell growth, such as cancer as described herein, it will be appreciated that it may also be used to identify disease-relevant markers relating to genes or gene products that do not directly affect FGFR4 gene or protein expression or activity, or the expression or activity of which is not directly affected by the FGR4 protein. It will be appreciated that the above-mentioned uses represent further aspects of the present invention. This may be done, e.g., by performing differential proteomics analysis, using techniques including , e.g., 2D-gel electrophoresis, protein chip microarrays, SILAC or mass spectrophotometry on tissue displaying uncontrolled cell growth, e.g., cancer, as described herein. This may also be done on nucleic acid level by, e.g., differential display or cDNA microarrays.

A further aspect of the invention is the use of the non-human animal, e.g. rodent, primary cells, or cell lines as described herein as a model for studying the molecular mechanisms of, or physiological processes or medical conditions associated with undesirable activity, expression, or production of said modified FGFR4. This may be done, e.g., by performing differential proteomics analysis, using techniques including, e.g., 2D-gel electrophoresis, protein chip microarrays, SILAC or mass spectrophotometry on tissue displaying uncontrolled cell growth, e.g., cancer, as described herein. This may also be done on nucleic acid level by, e.g., differential display or cDNA microarrays.

The term “undesirable activity, expression, or production of said modified FGFR4” as used herein refers to any undesirable activity, expression, or production of the protein and/or gene encoding said modified FGFR4. The undesirable activity, expression, or production may relate to any aberrant activity, expression or production, i.e., activity, expression or production beyond the normal activity, expression or production of FGFR4 as well as any activity, expression or production that is below the normal activity, expression or production of FGFR4.

It will also be appreciated that the non-human animals described herein, e.g., a rodent, as well as the primary cells, host cells, or cell lines as described herein, will be highly useful as a model system for the screening and identification of binding partners, particularly ligands of the FGFR4 protein, or upstream or downstream genes, or genes or proteins regulated by FGFR4 protein or its gene or protein activity and/or deregulated by expression of a modified FGFR4 or in disorders associated with modified FGFR4 protein. Such agents may be, for example, small molecule drugs, peptides or polypeptide, or nucleic acids, in particular such polypeptides described in Table 1 or Table 2 below. Particular preferred polypeptides are selected from the group consisting of protein tyrosine phosphatase receptor type F (PTPRF, LAR), the neurogenic locus notch homologue 2 (NOTCH2), the Ephrin type-A receptor 2 (EPHA2), the Epidermal growth factor receptor (EGFR) (SEQ ID NO: 77), β-Klotho, hydroxyacid oxidase 1, propanoyl-6AC-acyltransferase, formimideyltransferase-cyclodeaminase, and hydroxymethylglutaryl-6A synthetase. The most particularly preferred polypeptide is EGFR (SEQ ID NO: 77).

It will also be appreciated that the non-human animals described herein, e.g., a rodent, as well as the primary cells or cell lines as described herein, will be highly useful for studying whether the amino acid modifications of the FGFR4 in mammary tumors as described herein plays the same or a similar role in other cancer types, e.g., in hepatocelluar cancer, lung cancer, prostate cancer, colorectal cancer, melanoma, or in pancreatic cancer.

The invention further relates to a modified FGFR4 polypeptide and nucleic acid molecules, e.g., a gene, encoding such a modified FGFR4 protein or polypeptide as described herein in particular in connection with the animals.

Accordingly, the present invention also provides amino acid sequences of a modified FGFR4, for example, murine and rat modified FGFR4 amino acid sequences. The wild type murine and/or rat FGFR4 amino acid sequences are shown in SEQ ID NO: 1 and SEQ ID NO: 4, respectively. A preferred modified version of FGFR4, e.g., the mouse and/or rat FGFR4, amino acid sequence is one wherein glycine at position 385 or 386 is mutated to a non-glycine amino acid. A more preferred version of the mouse and/or rat FGFR4 amino acid sequence is one wherein glycine at position 385 or 386 is mutated to a charged amino acid, e.g., a positively charged amino acid, i.e., lysine, arginine, or histidine. A most preferred version of the mouse and/or rat FGFR4 amino acid sequence is one wherein glycine at position 385 or 386 is mutated to an arginine (SEQ ID NO: 5 or SEQ ID NO: 6).

Another preferred version of FGFR4 is one with a modification, e.g., an amino acid substitution, at at least one amino acid position compared to the wild-type FGFR4 protein, preferably an FGFR4 protein according to SEQ ID NO: 1, which modification, if present at least some or all or essentially all cells of a non-human animal of the invention, e.g. a rodent, in a heterozygous or homozygous manner, results in a phenotype associated with an alteration in tumor progression and/or formation as described herein, e.g., an increased rate of tumor growth and/or metastasis formation compared to a wild-type animal. In a preferred embodiment said animal additionally expresses in the genome of at least some of its cells a transgene encoding a TGF-α protein.

In one embodiment the modified FGFR4 protein and nucleic sequences as described herein are isolated protein or nucleic acid sequences. An “isolated” or “purified” polypeptide or protein, or a biologically active fragment thereof as described herein, is substantially free of cellular material or other contaminating proteins from the cell or tissue source from which the polypeptide or protein is derived, or substantially free from chemical precursors or other chemicals when chemically synthesized. The language “substantially free of cellular material” includes preparations of the FGFR4 protein in which the protein is separated from cellular components of the cells from which the protein is isolated or in which it is recombinantly produced.

Also encompassed by the present invention are fragments of such proteins comprising at least 6, at least 7, at least 8, at least 9, at least 10, at least 15, at least 20, at least 25, at least 30, at least 35, at least 40, at least 50, at least 60, at least 70, at least 80, at least 90, at least 100, at least 150, at least 200, at least 250, at least 300, at least 350, at least 400, at least 450, at least 460, at least 470, at least 480, at least 490, at least 500, at least 550, at least 600, at least 650, at least 700, at least 750, a least 790, at least 791, at least 792, at least 793, at least 794, at least 795, at least 796, at least 797, at least 798, at least 799 or at least 800 contiguous amino acids having the amino acid modifications as described herein, e.g., an amino acid substitution in the wild-type FGFR4, e.g., mouse or rat FGFR4, at the amino acid position corresponding to amino acid position 385 of SEQ ID NO: 1 or an an amino acid substitution at at least one amino acid position compared to the wild-type FGFR4 protein, preferably an FGFR4 protein according to SEQ ID NO: 1 or 386 of SEQ ID NO: 4, which modification, if present at least some or all or essentially all cells of a non-human animal of the invention as described herein, e.g. a rodent, in a heterozygous or homozygous manner, results in a phenotype associated with an alteration in tumor progression and/or formation as described herein, e.g., an increased rate of tumor growth and/or metastasis formation compared to a wild-type animal. In a preferred embodiment said animal additionally expresses in the genome of at least some of its cells a transgene encoding a TGF-α protein.

In a preferred embodiment, the protein of the invention represents an orthologue of the mouse FGFR4 protein according to SEQ ID NO: 5, preferably a vertebrate orthologue. Alternatively, it may represent a mammalian orthologue, in particular a rodent selected from the genus Mus (e.g., mice), Rattus (e.g. rat), Oryctologus (e.g. Rabbit) or Mesocricetus (e.g., hamster), preferably the rat orthologue according to SEQ ID NO: 6. It may also be a variant of the mouse FGFR4 protein according to SEQ ID NO: 5, respectively, or of said orthologue, preferably said rat orthologue according to SEQ ID NO: 6, allelic variant or otherwise, wherein certain amino acids or partial amino acid sequences have been replaced, added, or deleted.

Again in a preferred embodiment, the modification mentioned above results in a deletion or substitution by another amino acid of at least one an amino acid of said mouse FGFR4 protein according to SEQ ID NO: 1 or corresponding FGFR4. Alternatively, the modification may result in an insertion of additional amino acids not normally present in the amino acid sequence of the mouse FGFR4 protein or corresponding FGFR4 defined above.

The substitution may furthermore be a substitution of an amino acid by another amino acid, which is a conservative amino acid substitution between mouse and rat FGFR4 as described above. Such an amino acid may be a non-naturally occurring or a naturally occurring amino acid.

Preferably, the wild type residue of the modified FGFR4 protein is replaced by an amino acid with different size and/or polarity as defined above.

The invention furthermore encompasses mature modified mouse FGFR4 or rat FGFR4 proteins, or their vertebrate orthologues, e.g., the specific orthologues referred to above, which comprise an amino acid or amino acid sequences corresponding to a modification as defined herein.

The invention also provides modified FGFR4 based chimeric or fusion proteins. As used herein, a “chimeric protein” or “fusion protein” comprises a FGFR4 protein, either wild type or modified in accordance with the present invention, or a fragment of such protein as defined above, linked to a non-FGFR4 polypeptide (i.e., a polypeptide that does not comprise a FGFR4 protein or a fragment thereof), e.g., amino acid sequences that are commonly used to facilitate purification or labeling, e.g., polyhistidine tails (such as hexahistidine segments), FLAG tags, HSV-tags, a beta-galactosidase tags and streptavidin.

The amino acid sequences of the present invention may be made by using peptide synthesis techniques well known in the art, such as solid phase peptide synthesis (see, for example, Fields et al., “Principles and Practice of Solid Phase Synthesis” in SYNTHETIC PEPTIDES, A USERS GUIDE, Grant, G. A. , Ed., W. H. Freeman Co. NY. 1992, Chap. 3 pp. 77-183; Barbs, K. and Gatos, D. “Convergent Peptide Synthesis”in FMOC SOLID PHASE PEPTIDE SYNTHESIS, Chan, W. C. and White, P. D. Eds., Oxford University Press, New York, 2000, Chap. 9: pp. 215-228) or by recombinant DNA manipulations and recombinant expression, e.g., in a host cell. Techniques for making substitution mutations at predetermined sites in DNA having a known sequence are well known and include, for example, M13 mutagenesis.

Manipulation of DNA sequences to produce variant proteins which manifests as substitutional, insertional or deletional variants are conveniently described, for example, in Sambrook et al. (see below).

The present invention provides nucleic acid or gene sequences encoding the FGFR4 proteins as described in more detail above and below, for example FGFR4 modified in accordance with the (animals, e.g., rodents of the) present invention. In a preferred embodiment, this invention provides a nucleic acid sequence encoding a modified mouse and/or rat FGFR4 protein as described herein. Modified mouse and/or rat FGFR4 encoding nucleic acids or genes, can be made, for example, by altering codon 385 of the wild type mouse FGFR4 gene or codon 386 of the wild-type rat FGFR4, such that codon 385 or 386, respectively, no longer encodes glycine. The construction of a gene with a 385^{th} or 386^{th} codon, respectively, that does not encode glycine can be achieved by methods well known in the art.

Glycine is encoded by GGA, GGC, GGG, or GGT. A codon that does not encode glycine may be, for example, a codon that encodes Phe (TTT, TTC); Leu (TTA, TTG, CTT, CTC, CTA, CTG); Ile (ATT, ATC, ATA); Met (ATG); Asp (GAC, GAT); Ser (TCT, TCC, TCA, TCG), Val (GTT, GTC, GTA and GTG); Pro (CCT, CCC, CCA, CCG); Thr (ACT, ACC, ACA, ACG), Ala (GCT, GCC, GCA, GCG); His (CAT, CAC), Gln (CAA, CAG); Asn (AAT, AAC); Lys (AAA, AAG); Glu (GAA, GAG); Cys (TGT, TGC); Trp (TGG); Arg (CGT, COC, CGA, CGG, AGA, AGO); Ser (AGT, AGC); Tyr (TAC, TAT) or one of the stop codons (TAA, TAG, TGA). Again, methods for the introduction of site-specific nucleic acid mutations are well known.

Alternatively, at least one codon of the wild-type FGFR4 may be altered such that it encode another amino acid than the wild-type amino acid as long as the modification, if present at least some or all or essentially all cells of the animals of the present invention, e.g., a rodent, in a heterozygous or homozygous manner, results in a phenotype associated with an alteration in tumor progression and/or formation as described herein, e.g., an increased rate of tumor growth and/or metastasis formation compared to a wild-type animal. In a preferred embodiment said animal additionally expresses in the genome of at least some of its cells a transgene encoding a TGF-α protein.

The nucleic acid sequences or genes encoding the modified FGFr4 proteins, and fragments thereof, of the invention may exist alone or in combination with other nucleic acid sequences, for example within episomal elements, genomes, or vector molecules, such as plasmids, including expression or cloning vectors.

The term “nucleic acid sequence” as used herein refers to any contiguous sequence series of nucleotide bases, i.e., a polynucleotide, and is preferably a ribonucleic acid (RNA) ordeoxy-ribonucleic acid (DNA). Preferably the nucleic acid sequence is cDNA. It may, however, also be, for example, a peptide nucleic acid (PNA).

An “isolated” nucleic acid molecule or gene, as referred to herein, is one, which is separated from other nucleic acid molecules ordinarily present in the natural source of the nucleic acid. Preferably, an “isolated” nucleic acid or gene is free of sequences, which naturally flank the nucleic acid (i.e., sequences located at the 5′- and 3′-termini of the nucleic acid) in the genomic DNA of the organism that is the natural (wild type) source of the DNA.

FGFR4 gene molecules can be isolated using standard hybridization and cloning techniques, as described, for instance, in Sambrook et al. (eds.), MOLECULAR CLONING: A LABORATORY MANUAL (2nd Ed.), Cold Spring Harbor Laboratory Press, Cold Spring Harbor, N.Y., 1989 ; and Ausubel et al. (eds.), CURRENT PROTOCOLS IN MOLECULAR BIOLOGY, John Wiley & Sons, New York, N.Y., 1993.

A nucleic acid or gene of the invention can be amplified using cDNA, mRNA or, alternatively, genomic DNA, as a template and appropriate oligonucleotide primers according to standard polymerase chain reaction (PCR) amplification techniques. The nucleic acid or gene so amplified can be cloned into an appropriate vector and characterized by DNA sequence analysis. Furthermore, oligonucleotides corresponding to FGFR4 nucleotide sequences according to the invention can be prepared by standard synthetic techniques, e.g., using an automated DNA synthesizer.

Another aspect of the invention pertains to vectors, preferably expression vectors, containing a nucleic acid or gene encoding a modified FGFR4 protein, or derivatives, fragments, analogs, homologs or fusion proteins thereof. As used herein, the term “vector” refers to a nucleic acid molecule capable of transporting another nucleic acid to which it has been linked.

One type of suitable vector is a “plasmid”, which refers to a circular double stranded circular DNA molecule into which additional DNA segments can be ligated. Another suitable type of vector is a viral vector, wherein additional DNA segments can be ligated into a viral genome or parts thereof. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

A host cell of the invention, such as a prokaryotic or eukaryotic host cell in culture, can be used to produce (i.e., express) a modified FGFR4 protein as described herein.

Accordingly, the invention further provides a method for producing modified FGFR4 protein using the host cells of the invention. In one embodiment, the method comprises culturing the host cell of the invention (into which a recombinant expression vector encoding modified FGFR4 protein has been introduced) in a suitable medium such that modified FGFR4 protein is produced. In another embodiment, the method further comprises isolating modified FGFR4 protein, i.e., recombinantly produced protein, from the medium or the host cell.

The host cells of the invention can also be used to produce non-human transgenic animals. For example, in one embodiment, a host cell of the invention is a fertilized oocyte or an embryonic stem cell into which modified FGFR4 protein-coding sequences have been introduced. Such host cells can then be used to create non-human transgenic animals in which exogenous FGFR4 sequences have been introduced into their genome, or animals created by homologous recombination, in which endogenous FGFR4 sequences have been altered (see Examples, Material and Methods, below).

The (host) cell can also be used to identify agents that inhibit the interaction between a modified FGFR4 protein as described herein and any protein that acts as binding partner of a modified FGFR4.

In one embodiment the modified FGFR4 protein is a mouse FGFR4 protein, in which the glycine at position 385 of SEQ ID NO: 1 has been substituted by an amino acid different from glycine, particularly arginine, e.g. SEQ ID NO: 5.

In one embodiment the modified FGFR4 protein is a rat FGFR4 protein, in which the glycine at position 386 of SEQ ID NO: 4 has been substituted by an amino acid different from glycine, particularly arginine, e.g. SEQ ID NO: 6.

In one embodiment the modified FGFR4 protein is a human FGFR4 protein, in which the glycine at position 388 of SEQ ID NO: 2 or SEQ ID NO: 3 has been substituted by an amino acid different from glycine, particularly arginine.

In another aspect of the invention, the protein that acts as binding partner of a modified FGFR4 protein as described herein is protein tyrosine phosphatase receptor type F (PTPRF, LAR), the neurogenic locus notch homologue 2 (NOTCH2), the Ephrin type-A receptor 2 (EPHA2), the Epidermal growth factor receptor (EGFR) (SEQ ID NO: 77), β-Klotho, hydroxyacid oxidase 1, propanoyl-6AC-acyltransferase, form imideyltransferase-cyclodeaminase, and hydroxymethylglutaryl-6A synthetase. A particularly preferred protein in this regard is EGFR, e.g. of SEQ ID NO: 77.

For example, such a (host) cell can be used in a method of the invention, which is a method of identifying agents inhibiting the interaction between a modified FGFR4 as described above and any protein that acts as binding partner of a modified FGFR4 protein as described above, particularly EGFR protein, e.g., of SEQ ID NO: 77, comprising:
(a) culturing (a) cell(s) overexpressing a modified FGFR4 protein;
(b) adding the agent to be tested to the culture medium; and
(c) determining a decrease in the proliferation rate, an increase in apoptosis and/or a decrease in cell migration of the cell(s) overexpressing a wild-type FGFR4 protein cultured in the presence of the same agent.

In a preferred embodiment, the proliferation rate is determined via a MTT proliferation assay, the apoptosis is determined via FACS analysis (Nicoletti et al., 1991, J. Immunol. Methods, 139: 271-279), and/or the migration is determined with a Boyden Chamber Assay, e.g. as described in the Examples below.

A (host) cell in the context of the present invention, e.g. in the method described above, may be (a) MDA-MB-231 cell(s).

A further aspect of the present invention relates to an inhibitor of FGFR4 for the treatment of an EGFR-associated disorder, particularly of an EGF and/or TGF-alpha mediated disorder most particularly breast cancer or heptocellular cancer.

The inhibitor of FGFR4 may be an antibody directed against FGFR4, e.g., an antibody as used in the Examples provided below. In one embodiment the inhibitor is an aptamer directed against FGFR4. Preferably the aptamer is a single-stranded DNA- or RNA oligonucleotide of 25-70, 35-60, or 40-50 length. The production of aptamers is known by the skilled person, e.g., from Tuerk et al., 1990, Science 249: 505-510; Ellington et al., 1990, Nature 346: 818-822. Alternatively, the aptamer is a peptide aptamer. Such aptamers may consist of a variable loop of, e.g., 10-20 amino acids that is attached at both ends to a scaffold protein with good solubility properties, e.g., Thioredoxin A. Alternatively, the inhibitor is an antisense oligonucleotide directed against FGFR4. For example a single stranded DNA molecule that is complementary to the coding strand of the FGFR4 protein encoding mRNA.

Alternatively the inhibitor may be an RNAi molecule directed against FGFR4. Alternatively the inhibitor may be dominant negative mutant of the FGFR4 protein, e.g., the extracellular domain of FGFR4 protein.

FGFR4 may be FGFR4 protein or FGFR4 nucleic acid. It may be a mouse, a rat or a human FGFR4 protein as described herein, particularly a human protein of SEQ ID NO:2 or SEQ ID NO:3 or a nucleic acid sequence encoding said proteins. The FGFR4 may also be a modified human FGFR4 protein or nucleic acid as described herein, e.g., a mouse protein of SEQ ID NO:5, a rat protein of SEQ ID NO:6 or a human FGFR4 protein wherein the modification is an amino acid substitution of the amino acid glycine at the amino acid position 388 of SEQ ID NO:2 or SEQ ID NO:3 or a nucleic acid sequence encoding said proteins. Preferably the substitution of the amino acid is with arginine in said proteins or by a codon in the respective nucleic acid encoding said proteins.

The invention further pertains to a method of diagnosing severe cancer progression by

(a) determining the expression of EGFR gene or protein; and/or

(b) determining the interaction between FGFR4 protein and EGFR protein; and/or

(c) determining the stimulation of EGFR protein by TGF-alpha and/or EGF; and/or

(d) determining whether FGFR4 is the wild-type protein or gene, particularly of SEQ ID NO:2 or SEQ ID NO:3 or a modified human FGFR4 protein wherein the modification is an amino acid substitution of the amino acid glycine at the amino acid position 388 of SEQ ID NO:2 or SEQ ID NO:3, preferably a substitution with arginine

wherein an upregulation of the expression of EGFR gene or protein; an upregulation of the stimulation of EGFR protein by TGF-alpha and/or EGF; and/or the presence of said modified human FGFR4 protein is indicative for severe cancer progression.

The expression of EGFR gene or protein; the stimulation of EGFR protein by TGF-alpha and/or EGF; and/or the presence of said modified human FGFR4 protein are determine by methods known in the art, e.g., by the methods used in the Examples.

### EXAMPLES

Here we show in a genetically “clean” system the impact of a single nucleotide difference in the codon 385 of the mouse FGFR4 gene that converts a Glycine to an Arginine in the transmembrane domain of the receptor, on mammary cancer progression in vivo. We generated a FGFR4 Arg385 knock-in (KI) mouse model in order to investigate the effect of the two different FGFR4 alleles on breast cancer progression. For this purpose we crossed the FGFR4 Arg385 KI mice to WAP-TGFα/EGFR transgenic mice (19, 20). In this model, TGFα-overexpression is controlled by the whey acidic protein (WAP) promotor which specifically activates the transgene in mammary epithelial cells in mid-pregnancy (19). Thus, the process of mammary carcinogenesis is promoted by the constitutively high overexpression of TGFα, a ligand of the epidermal growth factor receptor (EGFR). Overexpression of TGFα in mammary epithelial cells results in accelerated alveolar development and impaired cell differentiation leading to failures in female lactation. Moreover, mammary involution is delayed and some alveolar structures fail to regress completely. As a consequence these hyperplasic alveolar nodules increase in number with successive pregnancies, and in some cases progress to tumors of variable histotype (20).

Here we report that the substitution from Glycine to Arginin at codon 385 enhances the progression of breast cancer in the WAP-TGFα mouse mammary carcinoma model in mass and size of the occurring tumors and this progression in vivo could be confirmed by in vitro data generated in mouse embryo fibroblasts. Moreover the FGFR4 Arg385 allele promotes lung metastasis in size and number of the occurring metastases.

These results spotlight the importance of the FGFR4 Arg385 allele in human breast cancer progression and may therefore serve as a prognostic marker of clinical outcome for patients affected by this disease.

Materials and Methods

Mouse Targeting Construct

The genomic sequence of the murine FGFR4 was detected in the RPCI mouse PAC library 21 of SV/129 genetic background (Celera, USA) by a specific cDNA-probe detecting exon 8-10. Exon 2-12 of the murine FGFR4 (12,5 kb) was then cloned into a pBS-vector (Stratagene, California) via a SpeI/SacII restriction (Biolabs, New England). Afterwards the G to A single nucleotide polymorphism (SNP) was introduced via specific mutagenesis in a subfragment of 320 bp, which was cloned into a pcDNA.3 vector (Invitrogen, USA) (21,22). This fragment containing the SNP was then recloned into the pBS-vector. The selection-cassette was finally integrated by ScaI (Biolabs, New England) restriction. Prior to electroporation of embryonic stem cells the targeting construct was linearised by SaII (Biolabs, New England) restriction.

Targeting of Embryonic Stem Cells (ES-cells) and Selection of Positive Clones

The ES-cell line E14 (23) was maintained on feeder-cells (i.e., irradiated mouse embryonic fibroblasts) in Dulbecco's modified Eagles Medium (DMEM, high glucose) containing 2 mM Glutamine, 1000 U/ml LIF, 0.1 mM β-Mercaptoethanol and 20% heat-inactivated foetal calf serum.

For transfection, 4×10⁷ cells were mixed with 100 μg of linearised targeting construct in PBS to a final volume of 800 μl. Electroporation (240V, 500 μF, 6 msec) was performed with a GenePulser (BioRad, Germany) and the cells were plated in 10 cm tissue culture dishes in DMEM containing 20% FCS, 2 mM Glutamin, 1000 U/ml Lif and 0.1 mM β-Mercaptoethanol.

On the next day the cells were selected with 200 μg/ml G418 for integration of the construct. Negative selection was done with 2 μM gancyclovir.

Resistant clones were analyzed by Southern blotting (24) for homologous and illegitimate recombination via a 5′ external-probe and a neomycin-specific probe, respectively. To generate chimeric mice, positive ES-cell clones were microinjected in C57BL/6 blastocysts and implanted into the uterus of a pseudo-pregnant recipient mother.

Mice and Genotyping

Chimeric mice were backcrossed to C57BL/6 mice to raise a founder generation with germline transition. For removal of the neoR-selection cassette mice were crossed with Deleter-Cre transgenic mice. Cre-deleter mice were again backcrossed to C57BL/6 mice to generate the first generation of the FGFR4 Arg385 knock-in (KI) mice. FGFR4 Arg385 KI mice were backcrossed at least ten times to C57/BL6 mice or five times to FVB mice. WAP-TGFα/EGFR transgenic 3.0 mice (20) were obtained by L. Henninghausen, NIH, Bethesda, USA in a mixed C57BI/6 and FVB genetic background and were backcrossed to C57BL/6 mice ten times. Mice were kept in the animal facility of the Max-Planck-Institute of Biochemistry under normal conditions.

MMTV-PymT transgenic mice were obtained from Christian Bader of the Max-Planck-Institute of Biochemistry in Munich in a SV/129 background (Guy et al., 1992).

Genotype was determined by PCR of genomic tail-DNA isolated using the Qiagen Blood & Tissue DNeasy Kit according to the manufacturer's recommendation. The removal of the selection cassette was detected using neoR-specific primers (5′-AGGATCTCCTGTCATCTCACCTTCCTCCTG-3′ and 5′-AAGAACTCGTCAAGAAGGCGATAGAAGGCG-3′). Removal of the Cre transgene was determined by Cre-specific primers (5′-AACATGCTTCATCGTCGG-3′ and 5′-TTAGGATCATCAGCTACACC-3′). Primer for detecting the genotype of the FGFR4 allele were specific for amplifying a 168 bp band spanning the FGFR4-SNP (forward: 5′-CGTGGACAACAGCAACCCCTG-3′; reverse: 5′-GCTGGCGAGAGTAGTGGCCACG-3′) with subsequent restriction of the amplification product via Mval restriction enzyme to distinguish the different FGFR4 alleles. The presence of the TGFα-transgene was confirmed by performing PCR analysis with TGF-α forward 5′-TGTCAGGCTCTGGAGAACAGC-3′ and reverse 5′-CACAGCGAACACCCACGTACC-3′ primers (primer sequence provided by L. Henninghausen, NIH, Berthesda, USA).

The presence of the PymT-transgene was confirmed by performing PCR analysis with PymT-forward 5′-TCG CCG CCT AAG ACT GC -3′ and reverse 5′-CCG CCC TGG GAA TGA TAG -3′.

Tumor Analysis

To analyse the occurring tumors, mice were sacrificed by cervical dislocation and opened ventrally. All mammary glands were excised for tumor-measurement. Tumor size and mass were analysed by metrical measurement and weighing of the tumor tissue and the mammary gland tissue independently. Raw-data were normalised to bodyweight. All data are shown as mean±SDM. All p-values were calculated using the paired students T-Test and values <0.05 were considered statistically significant.

Mouse Embryonic Fibroblasts (MEFs)

MEFs were isolated from 13.5 dpc embryos and kept in DMEM containing 10% FCS and maintained following the 3T3 protocol (25).

To stably overexpress EGFR, v-src and empty pLXSN, MEFs were selected for genomic integration with 0418 24 hrs after infection. Transmigration of MEFs was analyzed in Boyden Chambers (Schubert & Weiss, Germany). 1.5×10⁴ cells were seeded in starving medium containing 0% FCS. Migration was performed to DMEM containing 4% FCS for 16 h. Afterwards cells were stained with crystal violet and migrated cells were analyzed macroscopically. For quantification Boyden Chamber membranes were destained in 5% acidic acid and analyzed for staining intensity in the ELISA reader. For the Soft Agar Assay, cells (1×10⁵) were added to 3 ml of DMEM supplemented with 10% FBS and 0.3% agar and layered onto 6 ml of 0.5% agar beds in 60 mm dishes. After 24-96 h anchorage independent growth of cells was calculated and quantified microscopically. To perform a Matrigel Assay 5×10³ cells were seeded on Matrigel (BD bioscience)-coated 96-wells. After 24-96 h branching of cells was calculated and quantified microscopically.

To perform a proliferation assay, 1×10⁵ MEFs were seeded in 6 cm-dishes, maintained to 80% confluence, counted and re-seeded till senescence. The population doubling rate was determined by log (N/NO)×3,33 (N=cells at the end of growth period; N0=number of cells plated).

Senescence assays (Cell Signalling, USA) were performed on 1×10⁵ cells seeded in 6 cm-dishes. After 24 h cells were stained for β-galactosidase expression according to the manufacturer's recommendation and analysed under a light microscope (Visitron Systems, Zeiss).

The Focus Formation Assay was performed by infection (26) of MEFs with pLXSN (Clontech, Palo Alto, USA) based retroviruses containing the oncogenes v-src (positive control) HER2, EGFR or c-Kit. 24 h after infection cells were starved in medium containing 4% FCS and maintained for 21 days. Afterwards cells were stained with crystal violet and foci were counted macroscopically.

To calculate the life span and the population doubling rate of MEFs, 1×10⁵ MEFs were seeded in 6 cm-dishes, maintained to 80% confluence, counted and re-seeded till senescence. The population doubling rate was determined by log (N/NO)×3.33 (N=cells at the end of growth period; N0=number of cells plated).

Transmigration of MEFs were analysed in Boyden Chambers (Schubert & Weiss, Germany). 1.5×10⁴ cells were seeded in starving medium containing 0% FCS. Migration was performed to DMEM containing 4% FCS for 16 h. Afterwards cells were stained with crystal violet and migrated cells were analysed macroscopically. To perform an apoptosis assay 1.5×10⁴cells in DMEM containing 10% FCS were seeded in 12-well plates. After 24 h cells were treated with 0.5 μM doxorubicin for 48 h. Afterwards cells were stained with propidium-iodide and apoptotic cells were determined in a FACS (FACScalibus, BD) analysis as previously described (27).

RNA and Light Cycler® Analysis

Total RNA of minced murine tissues of adult mice was isolated using the RNeasy Kit (Qiagen, Germany) according to the manufacturer's recommendation. The quality of the isolated RNA was confirmed by agarose-gel electrophoresis visualising the 16S and 18S RNA. RNA was reverse transcribed into cDNA via the first strand cDNA kit of Boehringer Mannheim according to the manufacturer's protocol. The obtained cDNA was analysed via Light Cycler® Technology (Roche Diagnostics, Mannheim) for FGFR4 expression levels. Raw-data were normalised on expression levels of the housekeeping-gene HPRT and plotted as absolute values. Data are shown as mean±SDM.

Raw-data were normalized on expression levels of the housekeeping-gene HPRT and plotted relatively to the control that was set on 1 or 100%. Raw-data analysis via RT-PCR was quantified via ImageJ Software, normalized on the expression levels of the housekeeping-gene GAPDH and plotted relatively to the control that was set on 1 or 100%.

Immunoprecipitation and Western Blotting

For preparation of protein lysates, tumor samples were snap-frozen in liquid nitrogen, minced by an Ultratorax (Janke & Kunkel, IKA Labortechnik), lysed in RIPA lysis buffer containing phosphatase and proteinase-inhibitors for 30 min and precleared by centrifugation. Cultured cells were lysed in RIPA Buffer containing phosphatase and proteinase-inhibitors. For immunoprecipitation, lysates (1000 mg protein) were incubated with Protein A sepharose beads (GE Healthcare, San Francisco) and the according primary antibody (α-FGFR4 H121, Santa Cruz) at 4° C. over night. Afterwards samples were subjected for Western Blotting as previously described (28).

Raw-data analysis was quantified via ImageJ Software, normalized on the expression levels of actin/tubulin and plotted relatively to the control.

The following primary antibodies were used: FGFR4 sc9006 (Santa Cruz) (which is identical to α-FGFR4 H121), 4G10 (upstate), α-actin/α-tubulin (Sigma); secondary antibodies: α-rabbit-HRP conjugated (BioRAD) and α-mouse-HRP conjugated (Sigma)

Histology and Immunohistochemistry

Tumor samples and tissues were fixed in 70% Ethanol at 4° C. overnight. On the next day samples were embedded in paraffin and sections of 4-8 μM were cut on a microtome (HM355S, microm). The sections were subjected to deparaffinisation in xylene and rehydrated in a graded series of ethanol. Antigen retrieval was achieved by cooking in citrate buffer (pH 6) in a microwave. Immunohistochemical staining was done with the Vectastain Staining Kit (Vector Laboratories, Burlingame) following the manufacturer's protocol. After blocking with 10% horse serum in PBS buffer containing 3% Triton-X for one hour, the sections were incubated with the primary antibody (α-FGFR4 Hs121, Santa Cruz) at 4° C. overnight. The secondary antibody (α-rabbit, VectorLabs, USA) was incubated for one hour in PBS buffer containing 3% Triton-X. Mayer's Hematoxylin (Fluka, Switzerland) was used as counterstain.

For pathological analysis and quantitation of metastases, lungs were sectioned and analysed at 800 to 1000 μm intervals. Sections were stained with hematoxilin and eosin (H&E, Fluka, Switzerland) to identify lung metastases under the light microscope. Metastatic burden was calculated based on number and size of metastatic nodules.

Primers used
HPRT fw:
5′-ATA AGC CAG ACT TTG TTG GA-3′,
HPRT rev:
5′-CCA CTT GAA CTC TCA TCT TAG G-3′,
GAPDH fw:
5′-CCA ATA TGA TTC CAC CCA TGG-3′,
GAPDH rev:
5′-CCT TCT CCA TGG TGG TGA AGA-3′,
FGFR4 (for Light Cycler ®) fw:
5′-GCT TAT GGA TGA CTC CTT ACC CT-3′,
FGFR4 (for Light Cycler ®)rev:
5′-AA TGC CTC CAA TAC GAT TCT C-3′,
FGFR4 fw:
5′-CGT GGA CAA CAG CAA CCC CTG-3′,
FGFR4 rev:
5′-GCT GGC GAG AGT AGT GGC CAC G-3′,
E-Cadherin fw:
5′-GCT GGA CC GAGA GAG TTA-3′,
E-Cadherin rev:
5′-TCG TTC TCC ACT CTC ACA T-3′,
N-Cadherin fw:
5′-CCA CAG ACA TGG AAG GCA ATC C-3′,
N-Cadherin rev:
5′-CAC TGA TTC TGT ATG CCG CAT TC-3′,
MMP14 fw:
5′-CGT TCG CTG CTG GAC AAG G-3′,
MMP14 rev:
5′-GAC TGA GAA GGG AGG CTG GAG-3′,
MMP13 fw:
5′-TCC CTG GAA TTG GCA ACA AAG-3′,
MMP13 rev:
5′-GGA ATT TGT TGG CAT GAC TCT CAC-3′,
MMP9 fw:
5′-CCC TGG AAC TCA CAC GAC A-3′,
MMP9 rev:
5′-GGA AAC TCA CAC GCC AGA AG-3′,
CD44 fw:
5′-TTG AAT GTA ACC TGC CGC TAC GCA-3′,
CD44 rev:
5′-TCG GAT CCA TGA GTC ACA GTG CG-3′;
flk-1 fw:
5′-TCG TGC GTG ACA TCA AAG AG-3′,
flk-1 rev:
5′-TGG ACA GTG AGG CCA GGA TG-3′,
CDK4 fw:
5′-TGG CTG CCA CTC GAT ATG AAC-3′,
CDK4 rev:
5′-CCT CAG GTC CTG GTC TAT ATG-3′,
Rb fw:
5′-CAT CTA ATG GAC TTC CAG AG-3′,
Rb rev:
5′-CAT AAC AGT CCT AAC TGG AG-3′,
p21 fw
5′-CGT TTT CGG CCC TGA GAT GTT-3′,
p21 rev:
5′-ACC CGG GTC CTT CTT GTG TTT C-3′,
p53 fw
5′-AAC CGC CG ACCT ATC CTT ACC ATC-3′,
p53 rev:
5′-AGG CCC CAC TTT CTT GAC CAT TGT-3′,
cyclin D1 fw
5′-TCC CGC TGG CCA TGA ACT ACC-3′,
cyclin D1 rev:
5′-GGC GCA GGC TTG ACT CCA GAA-3′,
CDK1 fw
5′-CCA TGA ACT GCC CAG GAG-3′,
CDK1 rev:
5′-CGG TGT GGT GTA TAA GGG TAG A-3′,
CDK2 fw
5′-CGA TAA CAA GCT CCG TCC AT-3′,
CDK2 rev:
5′-AGA AGT GGC TGC ATC ACA AG-3′

Results:

Generation of FGFR4 Arg385 KI and FGFR4 Arg385 WAP-TGFα Transgenic Mice

Since an impact of the human FGFR4 Arg388 allele on tumor progression was just shown in correlative and partially controversial clinical studies there was an urgent need to ultimately clarify the influence of this single nucleotide polymorphism (SNP) on tumor progression in vivo. Here, the defined genetic background of a generated mouse model overcomes the heterogeneity of patient cohorts and thus the cause of resulting conflictive conclusions. Therefore, we generated a FGFR4 Arg385 knock-in (KI) model in the genetic background of SV/129 mice, which represents the first directly targeted KI mouse model to investigate the impact of a single nucleotide polymorphism on the progression of cancer. To achieve the genomic sequence of the murine FGFR4, a BAC-library was screened using a specific cDNA-probe detecting exons 8-10 of the FGFR4 gene and positive clones were analyzed by Southern blotting (data not shown). The gene targeting construct (FIG. 1A) contains exons 2-12 (12.5 kb) of the genomic sequence of the murine FGFR4. In order to generate the FGFR4 Arg385 allele, the glycine in exon 8 was changed to an arginine by site-directed mutagenesis. A neomycin selection cassette flanked by loxP sites was cloned between exons 10 and 11. After gene targeting upcoming neomycin-resistant ES-cell clones were analysed by Southern blotting (FIG. 1B1). Here a 5′-external probe was used to detect correct homologous recombination identifiable by an additional 10 kb band. The genotype of the positive ES-cell clones was then analysed by PCR-RFLP of the genomic DNA. Since the SNP inserts a new restriction site, the obtained amplification product in FGFR4 Arg385 carrying clones could be cut via the Mval restriction enzyme resulting in an additional 93 bp fragment (FIG. 1B2).

Next, positive clones were injected into blastocysts of pseudo-pregnant mice to generate chimeras. These mice were then backcrossed to C57BL/6 mice to raise the first generation of FGFR4 Arg385 KI mice. In order to delete the neomycin selection cassette, the FGFR4 Arg 385 mice were crossed to mice transgenic for the Cre-recombinase (Deleter-Cre).

FGFR4 Arg385 KI Cre-deleted mice were analysed by segregation analysis of a statistically significant number of mice for mendelian inheritance of the FGFR4 allele (FIG. 1C). In backcrosses to FGFR4 Gly/Gly385 mice, the offspring displayed the expected distribution of 1:1 from FGFR4 Gly/Gly385 to FGFR4 Gly/Arg385. Heterozygote intercrosses displayed the expected distribution of 1:2:1 from FGFR4 Gly/Gly385 to Gly/Arg385 to Arg/Arg385. Hence, the FGFR4 Arg385 allele is inherited in the correct mendelian ratio.

To investigate the impact of the FGFR4 Arg385 on mammary cancer progression, the FGFR4 Arg385 KI mice were crossed to mice transgenic for WAP-TGFα/EGFR (FIG. 1D3). To ensure normal lactation of female mice the transgene was only inherited by males. To confirm the presence of the TGFα transgene in the progeny we performed genotyping with specific primers for exogenous TGFα (FIG. 1D2). To distinguish the different FGFR4 alleles, the genotyping was done by PCR-RFLP by the aforementioned additional restriction site (FIG. 1D1).

FGFR4 Arg385 KI Mice Mimic their Human Counterparts

In humans, the FGFR4 Arg388 allele is expressed in various tissues without any difference compared to the FGFR4 Gly388 and has yet no known impact on the organism itself (11). Similarly, the FGFR4 Arg385 KI mouse model displays no yet known obvious phenotype that distinguishes the Arg385 from Gly385 carrying mice (data not shown). To check if the generated FGFR4 Arg385 KI mice mimic their human counterpart also in FGFR4 expression, localisation and distribution, we analyzed FGFR4 mRNA- and protein-levels and analysed the localisation and distribution in various tissues of adult mice.

As shown in FIGS. 2A and B, FGFR4 is expressed in various tissues including mammary gland, lung, brain or kidney. No difference between the different FGFR4 alleles on mRNA as well as on protein expression level is detectable. Next, we analysed the expression and localisation of FGFR4 in different tissues immunohistochemically (FIG. 2C). Here FGFR4 is detectable in various tissues and as with mRNA or protein expression-levels, there is no difference between the FGFR4 alleles. Two examples for FGFR4-stained tissues are given in FIG. 2D. In the lung, FGFR4 is expressed in smooth muscles, blood vessels and bronchial epithelial cells. In the mammary gland, blood vessels and ductal epithelial cells show a clear FGFR4 staining. These results are matching previously published data on human samples (29).

Hence the FGFR4 Arg385 KI mice seem to mimic their human counterparts in mRNA and protein expression levels, localisation and distribution.

FGFR4 Arg385 Promotes Tumor Progression

Previous reports of clinical studies do not implicate the FGFR4 Arg388 allele in tumor initiation, but rather associate it with enhanced disease progression once cancer has been initiated (11,12). Therefore we crossed the FGFR4 Arg385 KI mice to mice transgenic for WAP-TGFα/EGFR to initiate mammary tumors and to investigate for the first time the impact of the SNP FGFR4 Arg385 allele on mouse mammary tumor progression in vivo.

For this purpose we analysed the mass, area and percentage of the occurring tumors (FIG. 3A-D). Here, the tumor mass and area is significantly (Gly/Arg-p=0.03; Arg/Arg-p=0.002) increased (Gly/Arg-p=0.01; Arg/Arg-p=0.0006) in FGFR4 Arg385 (Arg385) carrying mice transgenic for WAP-TGFα when compared to FGFR4 Gly385 (Gly385) controls (FIGS. 3A and B). These results indicate that the FGFR4 Arg385 allele is a potent enhancer of TGFα-induced mammary tumors in mass and area.

Furthermore, the higher significance in the area of tumors suggests that the FGFR4Arg385 is not an enhancer of cancer cell proliferation, but seems to accelerate migration resulting in an increased invaded area of the mammary gland. Moreover, the more significant increase in tumor area may result from a facilitated neoplastic transformation rate in FGFR4Arg385 carrying mice transgenic for WAP-TGFα. These results are in line with the in vitro experiments in transformed MEFs. The analyzed control mammary glands of FGFR4Gly/Gly385, Gly/Arg385 and Arg/Arg385 mice without an oncogenic background do not display any changes in their mass, size or pathology (FIG. 10A-B).

Further we investigated the impact of the FGFR4 Arg385 allele on the initiation of mammary tumors in the WAP-TGFα/EGFR model. To do that we analyzed the amount of transformed mammary gland epithelia which is the percentage of mass and area of the tumor compared to the whole mammary gland (FIGS. 3C and 3D). Here the percentage of the tumor mass is significantly increased (Gly/Arg-p=0.05; Arg/Arg-p=0.005) whereas the percentage of tumor area shows a significant increase only when Gly/Gly and Arg/Arg (p=0.002) mice are compared. Thus, the FGFR4 Arg385 allele not only promotes tumor growth as shown in FIGS. 3A and B but seems to facilitate the initiation of TGFα/EGFR -induced mammary tumors since the percentage of tumorigenic mass and area increases from heterozygous to homozygous Arg385 carrying mice. Furthermore, the potent tumor enhancing impact of the FGFR4 Arg385 allele is in evidence by comparing an Arg/Arg 385 carrying mouse to a Gly/Gly control sacrificed after 6 month of mating and thereby tumor progression (FIG. 3E). In contrast, the inability of the FGFR4 to influence cancer initiation by itself is shown in FIG. 7A/B.

In addition to the WAP-TGFα mouse model we also investigated the tumor promoting impact of the FGFR4Arg385 allele in the MMTV-PymT mouse mammary carcinoma model. Because of the in vitro results in MEFs transformed with v-src we aimed to investigate if the tumor promoting action of the FGFR4Arg385 allele is likewise in vitro not apparent in vivo, to finally determine that the tumor enhancing effect of the FGFR4Arg385 is dependent on the oncogenic background.

Therefore we analyzed the tumors of 3 month old female FGFR4Gly/Gly385, Gly/Arg385 and Arg/Arg385 mice. The analyzed criteria for tumor progression are the mass and area of the analyzed tumors. As seen in FIGS. 11A and B there is neither a significant difference in tumor size nor in tumor mass between the FGFR4 isotypes in mice transgenic for MMTV-PymT. Thus, the tumor promoting effect of the FGFR4Arg385 in vivo allele is dependent on the genetic background, which triggers oncogenesis.

To further investigate the underlying mechanism of the tumor enhancing effect of the FGFR4 Arg385 allele, we studied possible molecular differences of the FGFR4 alleles. In many human cancers overexpression of the FGFR4 is a commonly observed feature of tumors (1, 4, 30, 31). To examine the FGFR4 expression in TGFα/EGFR -derived tumors we first analysed the expression by immunoprecipitation of the FGFR4 from tumor lysates of every genotype. Here, the FGFR4 protein is clearly overexpressed in tumors, when compared to non-cancerous mammary gland, however, there is no detectable difference between the different alleles (FIG. 3F).

Furthermore, we analysed the constitutive phosphorylation status of the FGFR4 to check if the Arg385 allele has any influence on the kinase activity and thereby leading to a tumor promoting effect. As shown in FIG. 3F, FGFR4 Arg/Arg385 seems to be more phosphorylated and thereby more activated then Gly/Gly- or Gly/Arg385, a possible hint of the tumor promoting potential of the FGFR4 Arg385 allele. The activation of downstream molecules like Erk or Akt did not show significant differences between the expressed FGFR4 alleles (data not shown). We also checked the expression levels of FGFR4 of every genotype in murine samples immunohistochemically (FIG. 3G). Remarkably, the expression of the FGFR4 alleles displays no differences in adenocarcinomas (FIG. 3G2) but show a clear increased expression of the Arg385 allele compared to Gly 385 in hyperplasic mammary glands (FIG. 3G1). Maybe the overexpression of FGFR4 Arg385 in oncogenesis is accelerated relative to the Glycine allele and thereby promotes tumor progression with an earlier onset. Additionally, the given examples of TGFα/EGFR-derived tumors (FIG. 3G) indicate no pathohistological differences due to FGFR4 Arg385 allele expression.

In addition to FGFR4 expression in primary tumors, we wanted to investigate the expression of genes associated with aggressive breast cancer parameters such as motility, invasivity and angiogenesis (FIG. 12). These were analyzed at the mRNA level in 6 month old tumors from FGFR4Gly/Gly385 and Arg/Arg385 mice transgenic for WAP-TGFα. Here, the expression in FGFR4Gly/Gly385 expressing WAP-TGFα-induced tumors was set on 100% and the expression in FGFR4Arg385 expressing WAP-TGFα-induced tumors was set relative to these expression levels. First, we analyzed the expression of the FGFR4 and EGFR to exclude that the tumor progressive impact results from the overexpression of the FGFR4Arg385 or the EGFR and to ensure, that these two proteins are equally expressed in the investigated mice. As seen in FIG. 12 both, the FGFR4 and the EGFR display no overexpression in the presence of the FGFR4Arg385 allele. Among tumor suppressors, the only significant alteration in expression was found for p21, which is significantly downregulated in FGFR4Arg385 expressing WAP-TGFα-induced tumors. This tumor suppressor is known to be a determinant for the poorest prognosis if its downregulated together with high EGFR expression. Regarding cell cycle and proliferation markers, the expression of the cell cycle dependent kinases (CDK) 1, 2 and 4 and Cyclin B were measured. As FGFR4 is known to have no strong mitogenic activity, no difference between FGFR4Gly385 or Arg385 expressing tumors was expected. In contrast, there was a significant higher expression of CDK1 in FGFR4Arg385 expressing tumors. As CDK1 is strongly associated with migration, this significant overexpression seems to promote an increase in the migratory action of the tumor cells resulting in a more aggressive phenotype of FGFR4Arg/Arg385 carrying tumors. In the group of invasion markers, the expression of proteins associated with metastasis and angiogenesis were analyzed. Here, the CD44 and flk-1 genes are significantly overexpressed in FGFR4Arg385 tumors. Although, the impact of CD44 on invasion is still controversial, however, its metastasis-promoting impact is widely accepted. The significant overexpression of flk-1 indicates a more aggressive potential of FGFR4Arg385 tumors as flk-1 promotes angiogenesis leading to a more aggressive behaviour of the tumor and its metastatic capacity. In the cluster of MMPs, MMP13 as well as MMP14 are overexpressed in FGFR4Arg385 tumors contributing to a higher metastatic potential.

These data strongly suggest a more aggressive behaviour of WAP-TGFα induced tumors expressing the FGFR4Arg/Arg385 resulting in an accelerated tumor progression.

FGFR4 Arg385 decreases Time Point of Tumor Incidence and Promotes Tumor Progression Over Time

To further analyse the tumor promoting effect of FGFR4 Arg385 we followed the tumor progression of all three genotypes over time in the WAP-TGFα/EGFR model.

First we checked the visible time point of tumor incidence. As shown in FIG. 4A the FGFR4 Arg385 (Gly/Arg385 and Arg/Arg385 were pooled) carrying mice significantly (p=0.001) develop tumors at earlier time points than the FGFR4 Gly385 controls. In addition, we further checked the time point of tumor onset in FGFR Arg385 KI mice compared to Gly-carriers induced by the WAP-TGFα transgen in the FVB background. Similarly, Arg allele-carrying mice display an earlier onset of tumors (p=ns) as shown in FIG. 8A.

Further, FGFR4 Gly/Arg385- or Arg/Arg385 mice establish not only a larger amount of tumors simultaneously, but importantly, increase their number of tumors over time faster than FGFR4 Gly/Gly385 mice (FIG. 4B). The tumor mass and area of FGFR4 Gly/Arg385 and Arg/Arg385-mice also progresses considerably faster than that of FGFR4 Gly/Gly385-mice (FIGS. 4C and D).

Remarkably, the FGFR4 Arg385 carrying mice show no difference at early time points of tumor progression, but mice homozygous for Arg385 show an immense acceleration of tumor growth after 6-8 months. Hence, a heterozygous FGFR4 Arg385 status seems to be sufficient for a decreased tumor incidence, but homozygous Arg385 carriers display an obvious faster progression once tumor formation occurs.

Next, we analysed the share of tumor mass and area in the mammary gland over time (FIGS. 4E and F). Here, both the percentage of tumor mass and size shows a clear increase from FGFR4 Gly/Gly385 to Gly/Arg385 to Arg/Arg385 mice. These data confirm the fact that the Arg385 allele seems to facilitate tumor initiation of WAP-TGFα/EGFR -induced breast tumors.

In summary, the FGFR4 Arg385 allele promotes breast tumor progression over time in number, mass and size of the occurring tumors and seems to facilitate the initiation of oncogenesis and thereby decrease the time point of tumor onset.

FGFR4 Arg385 Promotes Cancer Cell Metastasis

As clinical outcome of cancer is dependent on the invasive stage of the primary tumor it is essential to investigate the impact of the FGFR4 Arg385 allele on aggressiveness and invasiveness of WAP-TGFα/EGFR-derived tumors. Therefore we investigated the lungs of the dissected mice for the occurrence of distant metastases. Strikingly, FGFR4 Arg385 mice show an earlier incidence of lung metastases when compared to Gly385 mice (FIG. 5A) but do not differ pathohistologically (FIG. 5B). Beyond this, the FGFR4 Arg385 allele enhances lung metastases in both, size and number when compared to FGFR4 Gly/Gly385 to heterozygous to homozygous FGFR4 Arg385-carrying mice (FIG. 5C). The highest differences are visible in the number of micro metastases lower than 80 μm. These results suggest that the FGFR4 Arg385 allele seems to facilitate tumor cell invasion. Second, in metastases bigger than 360 μm, an evidence for an earlier incidence and a possible faster growth of metastases in FGFR4 Arg385 carrying mice is observed.

FGFR4 Arg385 Promotes Cellular Transformation

To further investigate the possible mechanism of the tumor progressive impact of the FGFR4 Arg385 allele on tumor progression in vitro we performed focus formation assays on isolated E13.5 mouse embryonic fibroblasts (MEFs).

The cells were transformed with several oncogenes to check whether the results obtained from the FGFR4 Arg385 KI mice are conform in vitro and whether there is a hint for the mechanism of the tumor progressive effect. MEFs expressing FGFR4 Gly/Gly385, Gly/Arg385 or Arg/Arg385 were infected with HER2, EGFR (SEQ ID NO: 77) or c-kit while v-src served as a positive control. As shown in FIG. 6A the number of foci in FGFR4 Arg385 MEFs is markedly increased in all three oncogenes suggesting that the FGFR4 Arg385 allele promotes cell transformation in cooperation with classical oncogenes. Remarkably, cell transformation by the EGFR or c-kit, receptors, which are normally regarded as weak oncogenes, lead to an unusual high number of foci. A possible explanation for this phenomenon may be that there is yet unknown crosstalk between FGFR4 Arg385 and other receptor tyrosine kinases.

Next to c-kit, the transformation of FGFR4Arg/Arg385 MEFs with EGFR displays a unusally high activity in the focus formation assay. Therefore, we aimed to investigate the involvement of the FGFR4Arg385 allele on several physiological processes after transformation of FGFR4Gly/Gly385 and Arg/Arg385 MEFs by stable overexpression of the EGFR (Seq ID NO: 76). Stable overexpression of v-src served as a positive control, stable overexpression of the empty pLXSN vector served as a negative control. To ensure equal expression among the infected MEFs, overexpression of EGFR and v-src were analyzed via immunoblot analysis and quantification. As shown in FIG. 13A, EGFR and v-src are equally expressed in FGFR4Gly/Gly385 and Arg/Arg385 MEFs. Interestingly, FGFR4 is clearly upregulated in EGFR transformed cells compared to v-src transformed MEFs and even more upregulated and hyperactivated in FGFR4Arg/Arg385 relative to Gly/Gly385 MEFs. This result is a possible explanation for the increased transformation rate in the focus formation assay of FGFR4Arg/Arg385 MEFs infected with EGFR viral expression vector. Moreover, the upregulation of the FGFR4 is a further indication for a so far unknown crosstalk of these two receptors. We further investigated if this upregulation of the FGFR4Arg/Arg385 compared to FGFR4Gly/Gly385 in MEFs transformed with EGFR influences certain biological processes. Regarding proliferation MEFs transformed with EGFR display no differences between the FGFR4 isotypes. Thus, the FGFR4Arg385 does not influence the proliferation of transformed MEFs (FIG. 13B). Next, we analyzed the influence of the FGFR4 alleles in MEFs transformed by EGFR on cell migration in Boyden Chamber assays. In contrast to non-transformed MEFs, FGFR4Arg/Arg385 MEFs transformed with EGFR display a significantly increased migratory capacity compared to Gly/Gly385 MEFs (FIG. 14A). Next to migration we analyzed the ability of transformed MEFs to survive without anchorage. This anchorage independent growth enables cancer cells to metastasize that in turn induces a more aggressive phenotype of the primary tumor. Therefore, we performed a soft colony formation assay with MEFs transformed by EGFR (FIG. 14B). MEFs transformed by EGFR overexpression display significantly accelerated anchorage independent growth after 24 and 96 hours by expressing the FGFR4Arg/Arg385. Since fully malignant cancer cells auquire the ability to degrade the extracellular matrix surrounding them to spread and invade the surrounding tissue, we wanted to analyze the impact of the FGFR4Arg/Arg385 on invasivity in a Matrigel assay (FIG. 14C). MEFs transformed by EGFR display significantly accelerated branching in matrigel after 24 and 96 hours in the presence of the FGFR4Arg/Arg385. In contrast, the biological processes including migration, soft agar colony formation and branching in Matrigel were not promoted by the FGFR4Arg385 MEFs transformed with v-src (FIG. 15).

These results demonstrate that the FGFR4Arg385 influences physiological processes in MEFs including migration, invasion and anchorage independence that all contribute to tumor progression. These processes are distinct from those affected by FGFR4Gly385 and, furthermore, the impact of the FGFR4Arg385 is dependent on the genetic background that triggers malignant transformation.

To mimic oncogenesis over time we performed an additional Focus Formation assay by terminating the focus formation at different time points (FIG. 6B). It is clearly shown that Arg/Arg-MEFs not only transform considerably faster, but also generate an increased number of foci. Hence the enhanced and more intense progression over time in vivo could be confirmed in vitro.

To support these observations by molecular analytical methods we determined whether the FGFR4 Arg385 allele is hyperactivated in MEFs and thereby enhances cell transformation. The expression of the different FGFR4 isoforms is equal in MEFs as well as its basic state of phosphorylation (FIG. 9A). Next, we wanted to check if FGFR4 Arg385 facilitates cell survival or influences physiological processes of MEFs. Therefore we analysed the number of population doublings until the cells enter senescence in vitro and additionally stained MEFs, subcultured for 30 days, for 6-galactosidase to visualize senescent cells. However, we found no obvious differences between the different FGFR4 alleles (FIG. 9B). Hence, the FGFR4 Arg385 allele does not seem to promote a prolonged cellular lifespan. Further we wanted to investigate the influence of the FGFR4 Arg385 allele on physiological processes. As a motility enhancing effect of is the FGFR4 Arg388 allele had already been shown by Bange and colleagues with a human mammary carcinoma cell line, we wanted to confirm this observation in MEFs, but as shown in FIG. 9C no difference observed when Gly/Gly—were compared to Arg/Arg-MEFs. Contrarily, Arg-carrying MEFs seemed to strongly support cell survival in response to DNA-damaging agents. After 48 h of treatment with doxorubicin, Arg/Arg-MEFs displayed a significantly (p=0.03) decreased percentage of apoptotic cells compared to Gly/Gly-MEFs. Additionally, we investigated the anti-apoptotic impact of the FGFR4 Arg385 after treatment with other chemotherapeutic drugs. In the presence of 3 μM cisplatin, that, similar to doxorubicin, intercalates with DNA, Arg-carrying MEFs show decreased apoptosis after 48 hrs of treatment. Contrarily, after 48 hrs of treatment with taxol, which interferes with the organisation of the mitotic spindle, no differences between Gly- or Arg-carrying MEFs were observed (FIG. 6C).

Maybe, the FGFR4 Arg385 allele enables the cells to survive DNA-damage by higher tolerance to chemotherapeutic drugs and DNA-damage or -repair systems respond faster and more effectively in MEFs expressing the FGFR4 Arg385 allele.

Investigation of New FGFR4 Interaction Partners

The most prominent influence of FGFR4 and its Arg388 variant is its implication in cancer correlating with a poor clinical outcome. Furthermore, FGFR4 is involved in the maintainance of liver homeostasis. However, the distinct mechanisms by which the FGFR4 supports oncogenesis or liver metabolism have yet to be elucidated. For that purpose, we performed a proteomic analysis of FGFR4 interaction partners by SILAC-based mass spectrometry in vitro and in vivo.

Investigation of New FGFR4 Binding Partners in MDA-MB-231 Cells

As the FGFR4 is expressed at rather low levels compared to e.g. HER-family receptors and the scientific tools like antibodies represent a limitation in the investigation of this receptor, we chose MDA-MB-231 breast tumor-derived cells modified by Bange et al. (2002) as model system. Here, FGFR4 is overexpressed either in its Gly388 or Arg388 variant and excerts its cancer progression accelerating effects (Bange et al. 2002 (35)). FGFR4 overexpression, extensively simplifies the detection of the FGFR4 protein via mass spectrometry and the differences between the FGFR4 alleles can be analyzed in the same model system.

To perform quantitative mass spectrometry analysis of FGFR4 interaction partners we used the SILAC Technology do achieve differerential metabolic labelling of the cells (Ong and Mann, 2006). To verify the obtained interaction partners we performed a so called “label switch”. Quantitative mass spectrometry was performed on MDA-MB-231 cells overexpressing either the Gly388 or Arg388 variant by Arg⁰/Lys⁰ as well as Arg¹⁰/Lys⁸ labels. Parental MDA-MB-231 cells expressing the empty pLXSN vector served as a negative control and were labeled Arg⁴/Lys⁶ (FIG. 16). Labelling of cells and sample preparation was done as previously described (Andersen et al., 2005; Shevchenko et al.).

Table 1 displays all proteins that are potential interaction partners of the FGFR4. Identified proteins were normalized to their detection value in MDA-MB-231 cells expressing the empty pLXSN. Therefrom, all proteins with a 5-fold upregulation compared to the negative control are putative interaction partners of the FGFR4. Table 1 further displays the intensity of interaction indicated by the upregulation compared to the negative control and the differences between the FGFR4 Gly388 and Arg388 variant at which the value 1 means no difference in interaction. The FGFR4 Gly388 and Arg388 themselfes were found to be highly upregulated as a result of the overexpression in MDA-MB-231 cells. These results indicate that the experimental setup as well as the overexpression system worked properly. Further, the protein tyrosine phosphatase, receptor type F (PTPRF, LAR), the neurogenic locus notch homolog protein 2 (NOTCH2), the Ephrin type-A receptor 2 (EPHA2) and most interestingly the Epidermal Growth Factor Receptor (EGFR, SEQ ID NO: 77) were found to be highly upregulated. LAR is a transmembrane phosphatase and is known to regulate the function of various receptor tyrosine kinases. Its activity is known to be negatively regulated by the EGFR (Ruhe et al., 2006). Loss of LAR is associated with increased hepatocyte cell proliferation by c-MET, insulin resistance and increased tumor cell metastasis (Machide et al., 2006; Mander et al., 2005; McArdle et al., 2005). Overexpression of LAR induces apoptosis in mammalian cells (Weng et al., 1998). Above that, LAR is implicated in the regulation of FGF-induced signalling by interacting with FRS2 (Wang et al., 2000). EPHA2 is a transmembrane receptor tyrosine kinase that is upregulated on many human aggressive cancer cells. Unlike other receptors, it displays kinase activity without ligand binding (EphrinA1) that causes tumor progression. In breast cancer cells, including MDA-MB-231, EPHA2 negatively regulates malignant cancer cell behavior upon ligand or antibody binding that induces cell adherence (Carles-Kinch et al., 2002; Noblitt et al., 2004).

EGFR overexpression in MDA-MB-231 cells is associated with several key features of cancer development and progression and represents a valid target in various cancers. In MDA-MB-231 cells, the stimulation of the EGFR via multiple mechanisms results in an increase of their malignant behavior (Wang et al., 2009; Zheng et al., 2009). These data indicate that MDA-MB-231 cells overexpressing the FGFR4 Gly388 or Arg388 variant present a useful model to study potential interaction partners of the FGFR4 in breast cancer cells. Furthermore, FGFR4 seems to interact with a variety of receptor tyrosine kinases. However, all potential interaction partners displayed no difference between the different FGFR4 isotypes.

TABLE 1
Summary of possible new interaction partners of the FGFR4 in MDA-MB-231 cells;
potential interaction partners were verified by the “lable switch”; evaluation criteria of identified
proteins were upregulation ≧5-fold, Razor Peptides (=RPs) > 2, PEP < 0.03; The table further
displays fold of upregulation and fold difference between the FGFR4 isotypes; value 1 implies
equal interaction between the FGFR4 isotypes

| Protein Names | Gene Names | RPs_1 | RPs_2 | PEP |
|---|---|---|---|---|
| Fibroblast growth factor receptor 4 | FGFR4 | 39 | 38 | 0 |
| Protein tyrosine phosphatase, receptor type, F | LAR | 3 | 3 | 4.0149E−27 |
| Epidermal growth factor receptor | EGFR | 2 | 3 | 3.9283E−37 |
| Ephrin type-A receptor 2 | EPHA2 | 5 | 5 | 1.1434E−22 |

| | | | | | | ratio | |
|---|---|---|---|---|---|---|---|
| | | ratio | | ratio | | FGFR4Arg388/ | |
| | | FGFR4Gly388 | | FGFR4Arg388 | | Gly388 | |
| Protein Names | Gene Names | (n = 2) | stdv | (n = 2) | stdv | (n = 2) | stdv |
| Fibroblast growth factor receptor 4 | FGFR4 | 26.42 | 7.97 | 36.19 | 17.71 | 1.26 | 0.04 |
| Protein tyrosine phosphatase, receptor type, F | LAR | 16.59 | 7.87 | 14.32 | 6.31 | 1.06 | 0.24 |
| Epidermal growth factor receptor | EGFR | 6.50 | 1.65 | 7.60 | 1.95 | 1.14 | 0.16 |
| Ephrin type-A receptor 2 | EPHA2 | 7.97 | 0.09 | 8.74 | 1.70 | 1.05 | 0.06 |

Validation of the EGFR/FGFR4 Interaction

Interestingly, the data obtained from the mass spectrometry analysis in MDA-MB-231 cells, displayed the EGFR amongst others as an interaction partner of the FGFR4. The EGFR is a key regulator of various processes in cancers, approved therapeutic target and the main component of tumor progression in the WAP-TGFα mouse mammary carcinoma model used in our experiments. Therefore, the validation of the potential interaction between the EGFR and the FGFR4 preceded the validation of the other analyzed interaction partners.

First we aimed to show, that the FGFR4 gets co-immunoprecipitated with the EGFR in MDA-MB-231 cells overexpressing either the empty pLXSN, pLXSN-Gly388 or -Arg388 (FIG. 17A). These data indicate a first hint for the interaction of these two receptors. In contrast to the mass spectrometry analysis, the Western Blot Analysis displayed an increased content of co-immunoprecipitated FGFR4 Arg388 compared to FGFR4 Gly388. As expected, the negative control displayed no co-immunoprecipitated FGFR4 as FGFR4 is barely expressed in MDA-MB-231 cells. Nevertheless, as proteins are mostly localized in clusters on the membrane, co-immunoprecipitation is no final evidence for an interaction of two receptors. Therefore, we investigated the EGFR-FGFR4 interaction upon EGF stimulation. As shown in FIG. 17B, the EGFR displays increased phosphorylation in the presence of the overexpressed FGFR4. Furthermore, the EGFR in MDA-MB-231 cells overexpressing the FGFR4 Arg388 is even more activated than in the presence of the FGFR4 Gly388. Interestingly, the co-immunoprecipitated FGFR4-Arg388 is more active than the FGFR4-Gly388. Above that, phosphorylation of the FGFR4 increases over time upon EGF stimulation. These data are confirmed by the quantification of the Western Blot Analysis (FIG. 17C) Furthermore, the activation of the downstream signalling protein Akt is increased in MDA-MB-231 cells overexpressing the FGFR4 Arg388 upon EGF stimulation. The activation of Erk did not differ between the different FGFR4 isotypes (data not shown). This result indicates a physiological interaction of the FGFR4 and EGFR upon EGF stimulation. Similarly, the EGFR-FGFR4 interaction is hardly seen in unstimulated cells. In summary, the FGFR4 and the EGFR are direct interaction partners. Here, FGFR4 seems to support EGFR induced signalling by receptor phosphorylation upon EGF stimulation, whereas the FGFR4 Arg388 enhances the signal.

To further confirm the data obtained in MDA-MB-231 cells we investigated the signalling upon EGF and TGFα stimulation in MEFs derived from the FGFR4 Arg385 KI mice transformed with EGFR. MEFs transformed with EGFR displayed an accelerated and prolonged activation of Akt in the presence of the FGFR4 Arg385 allele upon EGF and TGFα stimulation (FIG. 18A). The activation of Erk shows no difference between the different FGFR4 isotypes (data not shown). Similar to the MDA-MB-231 cells overexpressing the FGFR4 Arg388, MEFs transformed with EGFR and expressing the FGFR4 Arg385 display a significant increase in pEGFR levels compared to FGFR4 Gly385 MEFs (EGF5′-p=0.000073, EGF10′-p=0.0025, TGFα5′-p=0.07, TGFα10′-p=0.01) (FIG. 18B). Above that, MEFs transformed with EGFR display an activation of the FGFR4 upon EGF and TGFα stimulation (FIG. 18C). Similar to MDA-MB-231 cells, MEFs expressing the FGFR4 Arg385 allele display an increased activation of the FGFR4. These data confirm the results obtained in MDA-MB-231 cells. The FGFR4 Arg385 clearly supports the activation and following downstream signaling of the EGFR.

The FGFR4 Arg385 Influences the Migratory Behavior and the Sensitivity Towards Gefitinib in MDA-MB-231 Cells

To further investigate the interaction between the EGFR and the FGFR4, we analyzed the influence of the FGFR4 Arg388 on the biological properties of MDA-MB-231 cells. We firstly analyzed the proliferation of MDA-MB-231 cells overexpressing the empty pLXSN, pLXSN-Gly388 and -Arg388. As shown in FIG. 19A the overexpressed FGFR4 had no influence on the proliferation of MDA-MB-231 cells under normal conditions. As shown in FIG. 19B overexpression of the FGFR4 results in a tremendous increase in migration indicating the immense capacity of the FGFR4 to promote the migratory behavior of cells (Gly388-p=0.001, Arg388-p=0.001). Above that, MDA-MB-231 cells overexpressing the FGFR4 Arg388 display accelerated migratory behavior compared to MDA-MB-231 cells overexpressing the FGFR4 Gly388. In contrast to the data of Bange et al., FGFR4 Gly388 did not suppress the migration of MDA-MB-231 cells. This may be due to the scratch assay of Bange et al. (2002) (35) that possibly resulted in a different response compared to a Boyden Chamber Assay that monitors changes in chemotactic migration rather than cell-cell contact.

To further analyze the physiological connection between the EGFR and the FGFR4 we investigated the differences between the different FGFR4 alleles in MDA-MB-231 overexpressing cells upon exposure to Gefitinib. This small molecule tyrosine kinase inhibitor blocks EGFR phosphorylation by competing with ATP and thereby inhibits EGFR-mediated downstream signalling (Herbst et al., 2004). Therefore, physiological processes that require the dimerization of the EGFR and the FGFR4 should lead to different results in the presence of Gefitinib compared to those obtained without an EGFR inhibitor. We first determined the response of MDA-MB-231 cells either overexpressing the empty pLXSN vector or FGFR4 Gly388 or FGFR4 Arg388 towards increasing concentrations of Gefitinib (0.025-20 μM) in a MTT-proliferation assay (FIG. 20A). Interestingly, FGFR4 Arg388 expressing cells display a typical dose response curve whereas FGFR4 Gly388 and empty pLXSN vector expressing cells display no response up to 20 μM of Gefitinib. The analyzed IC₅₀ was estimated to be 18.72 μM for both, MDA-MB-231 cells expressing the empty pLXSN or FGFR4 Gly388. In contrast, the calculated IC₅₀ for MDA-MB-231 cells overexpressing the FGFR4 Arg388 allele was 9.53 μM. These results indicate a higher sensitivity of MDA-MB-231-FGFR4Arg388 cells towards Gefitinib and suggest a higher EGFR-dependence of these cells. Further, we wanted to determine if the decreased proliferation results from a proliferative stop or apoptosis induced by Gefitinib. Therefore, we investigated the impact of FGFR4 Arg388 overexpression on apoptosis in response to Gefitinib treatment in MDA-MB-231 cells. As shown in FIG. 20B FGFR4 Arg388 expressing MDA-MB-231 cells display a significantly increased apoptotic response towards Gefitinib after 96 hours compared to MDA-MB-231 s cells expressing the FGFR4 Gly388 (20 μM-p=0.012; 10 μM-p=0.0022). These data indicate that MDA-MB-231 cells expressing the FGFR4 Arg388 allele display an increased sensitivity towards Gefitinib regarding cellular survival. As MDA-MB-231 cells acquired a significantly accelerated migratory capacity by overexpressing the FGFR4 Arg388 allele we determined the migratory behavior of MDA-MB-231 cells in the presence of Gefitinib (2.5 μM) (FIG. 20C). After 15 hours of migration in Boyden Chamber Assays, MDA-MB-231 cells expressing the FGFR4 Arg388 allele display 22.28% inhibition of migration compared to the DMSO treated control cells. In contrast, MDA-MB-231 cells overexpressing the FGFR4 Gly388 allele displayed only 6.28% of inhibition. This result indicates that the migratory capacity of MDA-MB-231 cells overexpressing the FGFR4 Arg388 is dependent on the molecular action of the EGFR and furthermore displays an increased response towards Gefitinib treatment.

In conclusion, the treatment of MDA-MB-231 cells with Gefitinib suggests a strong physiological connection between FGFR4 and EGFR regarding cellular survival and migration. Above that, the dependence of the molecular interaction between FGFR4 and EGFR is increased in the presence of the FGFR4 Arg388 allele.

Investigation of New Interaction Partners of the Hepatic FGFR4 In Vivo

Stable isotype labelling in cell culture (SILAC) has become a versatile tool for quantitative, mass spectrometry (MS)-based proteomics. In order to investigate global interactions and connections tissue-specifically and with the impact of an whole organism Kruger et al. established an in vivo SILAC by feeding mice with a diet containing either the natural or the ³⁷C₆-substituted version of lysine (FIG. 21).

The FGFR4 is involved in various metabolic processes in the liver including lipid-, glucose- and bile acid metabolism as well as in liver carcinogenesis (Huang et al., 2008; Huang et al., 2007). Also recent publications provide some evidence for the molecular action of the FGFR4 and its Arg388 variant the distinct mechanism including interaction partners is still unknown (Stadler et al., 2006; Wang et al., 2006; Wang et al., 2008).

Quantitative Analysis of Hepatic FGFR4 Binding Partners and their Differences Regarding the FGFR4 Isotypes

In order to investigate novel interaction partners of the hepatic FGFR4, a mass spectrometry analysis was performed to identify all proteins co-immunoprecipitated with the FGFR4. To allow a quantifiable analysis of the interaction partners the labelled SILAC-mouse was used as an internal standard (Kruger et al., 2008). To exclude unspecific binding partners the first experimental step was to establish FGFR4 blocking peptides to selectively block the antibody-FGFR4 interaction to identify all unselective binders. As seen in FIG. 22A a FGFR4 overexpressing construct that was used to generate the homemade α-FGFR4ex antibody (C. Stadler, 2005) was transfected in HEK293 cells. The recombinant FGFR4 protein was purified and digested with either Trypsin or LysC. The obtained blocking peptides were tested in a FGFR4 immunoprecipitation for their blocking efficacy. As shown in FIG. 22A especially the tryptic digest of the FGFR4 blocking peptides clearly diminished the antibody-FGFR4 interaction. Therefore, the synthesized blocking peptides were applicable for the following mass spectrometry analysis of novel FGFR4 interaction partners in the liver.

FIG. 22B displays the experimental setup regarding the investigation of novel FGFR4 interaction partners via in vivo SILAC. The SILAC mouse was used as an internal standard to achieve quantifiable results. The hepatic FGFR4 of the unlabelled mouse was immunoprecipitated in the presence of the blocking peptides to detect unspecific binding partners. In the quantitative LC-MS/MS analysis FGFR4 and its specific interaction partners should be highly upregulated in the labelled fraction. Unspecific interaction partners should display a 1:1 ratio compared to the unlabelled fraction incubated with the blocking peptides. Although the blocking peptides displayed a high efficacy in the Western Blot analysis, mass spectrometry analysis detected ˜300 proteins as specific binding partners of the FGFR4 (data not shown). Such a high number of binding partners can not be a result of physiologically relevant interactions. Therefore, quantitative mass spectrometry analysis of hepatic FGFR4 interaction partners can not be performed with the blocking peptides employed in these experiments. In order to improve the specificity of the blocking reaction, we sequenced the obtained blocking peptide mixture to synthesize specific blocking peptides (FIG. 22C). In contrast to the blocking peptide mix obtained from the tryptic digest, all of the synthesized blocking peptides were inactive in the Western Blot analysis (data not shown). For that reason, the investigation of hepatic FGFR4 interaction partners was done with the liver of FGFR4 KO mice (Yu et al., 2000). FIGS. 22D and E shows the experimental setup to identify interaction partners of the hepatic FGFR4 and their differences between the FGFR4 isotypes.

Table 2 displays all identified FGFR4 isotype interaction partners. Here, significance (PEP<0.03), amount of razor peptides (RPs, >1) and an upregulation of at least 3 fold in FGFR4 KO experiments identified potential FGFR4 interaction partner. FGFR4 is highly upregulated in SILAC mice compared to FGFR4 KO mice. Therefore, the experimental workflow displays proper settings for the investigation of hepatic interaction partners of the FGFR4. Furthermore, the FGFR4 is not differentially expressed between the FGFR4 isotypes, a fact that was already shown by the characterization of the FGFR4 Arg385 KI mice. βKlotho is a known high affinity interaction partner of the FGFR4. This single-transmembrane protein is the essential co-receptor for the activation of downstream signaling events upon FGF19/15 stimulation of the FGFR4 . Therefore, the identification of βKlotho as a strong interaction partner was the “positive control” in the MS-analysis. As seen in Table 2 βKlotho is highly upregulated in SILAC mice compared to FGFR4 KO mice indicating yet again proper experimental settings. Besides that, the in vivo SILAC analysis of our mice yielded so far unknown interaction partners that could contribute to the elucidation of the molecular action of the FGFR4 and its Arg385/388 variant. Hydroxyacid-oxidase 1 (Hao1) is a mainly peroxisomal protein that oxidizes glycolate and glyoxycolate with a subsequent production of H2O2 and is primarily expressed in the liver and pancreas. Downregulation of Hao1 in rats results amongst others in the upregulation of proteins associated with oxidative stress (Recalcati et al., 2003). Propanoyl-CoA C-acetyltransferase (Scp2) plays an important role in the intracellular movement of cholesterol and possibly other lipids. Its deficiency results in multiple phenotypes in humans (Ferdinandusse et al., 2006). In mice loss of Scp2 induces alterations in the biliary lipid secretion and hepatic cholesterol metabolism (Fuchs et al., 2001). Formididoyl-transferase-cyclodeaminase (Ftcd) is suggested to control folic acid liver metabolism (Bashour and Bloom, 1998). Furthermore, Ftcd is recognized as a liver specific antigen that is detected in sera of patients with autoimmune hepatitis (Lapierre et al., 1999). Above that, Ftcd is overexpressed in hepatocellular carcinoma (HCC) and is therefore suggested to contribute to the diagnosis of early stage HCC (Fuchs et al., 2001). Hydroxymethylglutaryl-CoA-synthase (Hmgcs2) is a key regulator of keton body production and is highly expressed in liver and colon. It is known that Hmgcs2 is transcriptionally regulated by c-myc and FKHRL1, a member of the forkhead in rhabdomysarcoma family that represses the transcription of Hmgcs2 in HepG2 cells upon insulin stimulation. Furthermore, Hmgcs2 is implicated in colon cancer via its downregulation (Camarero et al., 2006; Nadal et al., 2002). Among these potential interactors Hao1 and Scp2 display stronger interaction with the FGFR4 Arg385 variant indicated by a higher ratio compared to the FGFR4 Gly385. All afore mentioned potential interaction partners are not yet implicated in tyrosine kinase signalling or known to interact with RTKs. Therefore, fundamental follow-up experiments are necessary to first put these proteins into the context of the molecular action of receptor tyrosine kinases. Next to these potential new interactors the most interesting target is the epidermal growth factor receptor (EGFR). The EGFR was found to significantly interact with the FGFR4 and furthermore has a higher affinity to the FGFR4 Arg385 isotype. Besides others, the EGFR-RAS-MAPKK axis is one of the most important pathways for cell proliferation in liver (Llovet and Bruix, 2008). These data show various new interaction partners of hepatic FGFR4. The direct interaction with the FGFR4 and their involvement in FGFR4-mediated signalling should be the subject of further investigations.

TABLE 2
Listing of identified interaction partners of hepatic FGFR4 and their differences between
the FGFR4 isotypes; List displays razor peptides of identified protein (RPs), protein and gene
names, protein IDs and their significance (PEP < 0.03); furthermore, the list displays the intensity of
the interaction partners and their differences between the FGFR4 alleles

| | | | | | | Gene | | |
|---|---|---|---|---|---|---|---|---|
| RPsKO_1 | RPsKO_2 | RPsGly385_1 | RPsArg385_1 | RPsArg385_2 | Protein Names | Names | Protein IDs | PEP |
| 3 | 4 | 7 | 6 | 5 | Beta-klotho | Betakl | IPI00118044; IPI00473391 | 5.68E−158 |
| 3 | 3 | 5 | 4 | 3 | Hydroxyacid oxidase 1 | Hao1 | IPI00123750 | 1.29E−73 |
| 4 | 4 | 5 | 6 | 4 | Fibroblast growth | Fgfr4 | IPI00742377; IPI00761669; | 1.89E−138 |
| | | | | | factor receptor 4 | | IPI00129219; IPI00473948; | |
| | | | | | | | IPI00473231 | |
| 12 | 13 | 8 | 5 | 6 | Propanoyl-CoA C- | Scp2 | IPI00134131; IPI00648476; | 2.61E−180 |
| | | | | | acyltransferase | | IPI00648007 | |
| 2 | 2 | 3 | 5 | 3 | Epidermal growth | Egfr | IPI00121190; IPI00411099; | 4.76E−29 |
| | | | | | factor receptor | | IPI00357770; IPI00122341; | |
| | | | | | | | IPI00229006; IPI00626433 | |
| 7 | 3 | 4 | 6 | 2 | Formimidoyl- | Ftcd | IPI00129011 | 2.41E−75 |
| | | | | | transferase- | | | |
| | | | | | cyclodeaminase | | | |
| 9 | 6 | 4 | 3 | 7 | Hydroxy- | Hmgcs2 | IPI00420718 | 3.27E−274 |
| | | | | | methylglutaryl- | | | |
| | | | | | CoA synthase | | | |

| Gene | | ratio FGFR4 | stdv FGFR4 | ratio FGFR4 | ratio FGFR4 | stdv FGFR4 | ratio FGFR4 |
|---|---|---|---|---|---|---|---|
| Names | Protein Names | KO (n = 2) | KO (n = 2) | Gly385 (n = 1) | Arg385 (n = 2) | Arg385 (n = 2) | Arg385/Gly385 |
| Betakl | Beta-klotho | 28.2 | 2.88 | 3.2 | 2.4 | 0.96 | 0.7 |
| Hao1 | Hydroxyacid oxidase 1 | 19.7 | 4.02 | 0.1 | 1.6 | 1.48 | 27.8 |
| Fgfr4 | Fibroblast growth factor | 12.9 | 9.33 | 1.5 | 1.3 | 0.22 | 0.8 |
| | receptor 4 | | | | | | |
| Scp2 | Propanoyl-CoA C- | 7.8 | 1.20 | 0.2 | 3.6 | 2.89 | 18.6 |
| | acyltransferase | | | | | | |
| Egfr | Epidermal growth factor | 5.8 | 1.23 | 0.3 | 1.3 | 0.31 | 3.8 |
| | receptor | | | | | | |
| Ftcd | Formimidoyltransferase- | 3.5 | 0.22 | 0.4 | 1.0 | 0.52 | 2.7 |
| | cyclodeaminase | | | | | | |
| Hmgcs2 | Hydroxymethylglutaryl-CoA | 3.3 | 0.29 | 1.8 | 1.8 | 0.43 | 1.0 |
| | synthase | | | | | | |

Discussion

In this study we investigated for the first time the impact of the receptor tyrosine kinase FGFR4 alleles at codon 385 on the initiation and progression of breast cancer in vivo. The FGFR4 Arg385 KI per se mimics its human counterpart in expression, localisation and distribution of the FGFR4 and displays no yet known obvious phenotype. To investigate the role of the FGFR4 Arg385 knock-in mouse in the progression of mammary carcinoma we crossed the FGFR4 Arg385 mice to mice transgenic for WAP-TGFα/EGFR. We show that the FGFR4 Arg385 allele directly promotes occurring TGFα-induced mammary tumors in mass and size. In addition, these tumors also increase over time depending on the different FGFR4 genotypes. Furthermore, it decreases the visible time point of tumor incidence and therefore seems to facilitate tumor initiation which is demonstrated by a higher percentage of tumorigenic mass and size in the progression over time.

Remarkably, the FGFR4 Arg385 allele not only promotes aggressiveness but also supports invasiveness of lung metastases. The time point of metastases is substantially decreased and the lungs in FGFR4 Arg385 carrying mice are more invaded than in control animals.

These data strongly associate the FGFR4 Arg385/388 allele with poor prognosis and thereby highlight the receptor as a possible marker of breast cancer progression. Our in vivo results are in line with several clinical reports that were published since the discovery of the FGFR4 Arg388 allele by Bange and colleagues, which associate the FGFR4 Arg388 allele with tumor progression in various cancers like head and neck, prostate or breast (12-14).

In addition, our data in mice could be confirmed in vitro. Mouse embryonic fibroblasts carrying the Arg385 allele showed a higher transformation rate than control fibroblasts when infected with different oncogenes in a Focus Formation Assay. Furthermore, we could show a clear increase in number and growth rate of transformed foci in Arg385 MEFs over time.

Next to c-kit, the focus formation through the overexpression of the EGFR resulted in a very high number of foci. Therefore, we wanted to investigate if the FGFR4Arg385 allele contributes to EGFR driven transformation. To this end, we stably transformed the MEF FGFR4 genotype variants by EGFR overexpression. Interestingly, FGFR4 was upregulated in EGFR transformed MEFs and the FGFR4Arg385 was found to be hyperactivated in MEFs transformed with EGFR compared to FGFR4Gly385. These results indicate a possible crosstalk between these two receptors as it has been shown for HER2 and FGFR4. In EGFR-transformed MEFs, the FGFR4Arg385 isotype was significantly associated with accelerated cell motility, soft agar colony formation and branching in matrigel. These data indicate that FGFR4Arg385 promotes cell transformation through processes connected to migration and invasion. Furthermore, as a migratory effect is not detectable in non-transformed MEFs, these data clearly indicate that the FGFR4Arg385 is not an oncogene per se, but rather supports oncogenes by the enhancement of relevant physiological processes. Moreover, no impact of the FGFR4Arg385 could be detected when MEFs were transformed with v-src. These results suggest that the impact of FGFR4Arg385 is clearly dependent on the oncogenic background that triggers the neoplastic transformation and indicates a supportive rather than autonomous action of the FGFR4Arg385 isotype.

But for all that, the molecular mechanism of the tumor progressive impact was still unknown. Our study provides some evidence that may be an explanation for the tumor progressive function of the FGFR4 Arg385 allele. First, the FGFR4 Arg385 allele was more activated in analysed tumors when compared to the other alleles probably leading to a more intensive signalling and further to a higher cell proliferation and tumor progression. Second, the unusual number of foci in c-kit- and EGFR-induced Focus Formation Assays. The FGFR4 Arg385 allele enables additional unknown crosstalk to other receptors and their downstream signalling is molecules which can drive tumor progression. Third, the Gly385 allele was described to have a suppressive function (33). In this context it may be that the Arg385 allele fails to achieve this suppression and thereby releases or potentiates signalling of other receptor tyrosine kinases. Mass spectrometric analysis was used to define either additional binding partners and adaptor proteins or additional downstream-molecules, which get activated after specific stimulation of the different FGFR4 alleles.

Furthermore, we analyzed the molecular consequences of FGFR4Arg385 isotype expression in tumors to investigate the underlying mechanism of accelerated tumor progression. Although FGFR4Arg385 is not generally overexpressed in primary tumors relative to FGFR4Gly385 its activity is upregulated. As the amino acid substitution in the FGFR4 results in the conversion to a hydrophilic amino acid, the structure of the FGFR4Arg385 possibly impairs the binding of negative regulators to the kinase domain or alternatively allows activators to bind with higher affinity. For example Wang and colleagues (36) demonstrated increased stability of the FGFR4Arg388 receptor in prostate cancer cell lines . This delayed internalization of FGFR4Arg385 could be a result of an altered structure resulting in a relatively higher phosphorylation status. Furthermore, two studies identified changes in the cellular gene expression profile in the presence of FGFR4Arg388.

Here, FGFR4Arg388 promotes the upregulation of the metastasis-associated gene Ehm2 in prostate cancer and the pro-migratory gene LPA receptor EDG-2 in MDA-MB-231 cells that is suppressed by the FGFR4Gly388 and, interestingly, is a well known transactivator of the EGFR. Here, a micro array analysis of WAP-TGFα derived tumors could help to investigate differences in the FGFR4 isotype-induced gene expression profile in cancer cells. In this study, we analyzed the expression of several genes involved in tumor progression. Here, FGFR4Arg385 carrying WAP-TGFα-derived tumors display a more “aggressive” gene expression pattern. The significant downregulation of the tumor suppressor p21 is known to predict the poorest prognosis together with high EGFR expression and the upregulation of cell cycle dependent kinase (CDK) 1 involves the FGFR4 in an enhanced migratory capacity of cancer cells. The unchanged expression of the other cell cycle proteins confirms the lack of involvement of FGFR4Arg385 in cell proliferation. Moreover, genes associated with cell invasivity were upregulated in FGFR4Arg385 expressing WAP-TGFα-derived tumors. For instance, CD44 promotes metastasis formation, likewise the VEGF receptor flk-1 that regulates tumor angiogenesis. Accordingly, MMP13 as well as MMP14 are significantly overexpressed in FGFRArg/Arg385 expressing tumors contributing to a higher metastatic potential.

Besides changes in gene expression, FGFR4 isotypes could differ in their affinity towards other functionally relevant proteins. To address this possibility we performed a SILAC based mass spectrometry analysis of immunoprecipitates of the FGFR4Gly388 and Arg388 in the MDA-MB-231 breast cancer cell line model. Here, we identified the EGFR as a strong interaction partner of the FGFR4. Subsequent experiments interestingly showed a significantly higher affinity of the EGFR to FGFR4Arg388 variant resulting in enhanced downstream signalling. This interaction may likely be the key mechanism of the tumor progression promoting effect of the FGFR4Arg388 which is supported by our results in the KI WAP-TGFα mouse model in which a hyperactive EGFR drives mammary carcinogenesis. Besides that, the transformation assay in MEFs expressing the FGFR4 Arg385 displayed an unusual high number of foci by transformation with c-kit. These data indicate that next to the potential interaction partner EGFR, further receptor tyrosine kinases and oncogenes possibly crosstalk to the FGFR4 with a stronger affinity to the FGFR4 Arg388/385. These findings should be the subject of further investigations to finally determine the involved interaction partners of FGFR4 signalling and the differences regarding the FGFR4 isotypes.

Consistent with the gene expression differences and our preliminary EGFR interaction hypothesis, mouse cancer cells expressing the FGFR4Arg385 allele display an enhanced potential in invading the lung to form distant metastases in vivao. We observed that metastasis formation sets in earlier and with a significantly increased number of pulmonary metastases when compared to mice transgenic for WAP-TGFα expressing FGFR4Gly385. These data strongly associate the FGFR4Arg388 allele with poor prognosis and thereby highlight this receptor as a marker of breast cancer progression. Our in vivo results are in line with several clinical reports, that were published since the discovery of the FGFR4Arg388 allele by our laboratory, which associate the FGFR4Arg388 allele with tumor progression in various cancers like those of the head and neck, prostate, breast, melanoma and others.

In contrast, FGFR4Arg385 was not able to promote mammary cancer progression in mice transgenic for MMTV-PyMT neither in tumor mass or area. However, the negative results in the MMTV-PyMT-model presents an indirect evidence of a cancer cell specific action of the FGFR4 as it should promote mammary tumor progression induced by MMTV-PyMT if the cancer promoting effect would be indirect. This is well in line with the results obtained with MEFs stably transformed with v-src. In this case, FGFR4Arg385 could not enhance any of the analyzed biological properties. These findings underline the dependency of the impact of the FGFR4Arg385 isotype on the specific oncogenic background of neoplastic transformation. While the WAP-TGFα induced tumors include a hyperactive EGFR, the PyMT activates src leading to tumor formation. As analyzed by mass spectrometry, EGFR is a direct interactor of the FGFR4 with a stronger affinity to the Arg388/385 variant. This interaction seems to be the explanation for the different results in the WAP-TGFα—compared to the MMTV-PymT model.

Exceptionally, we could show that Arg-carrying MEFs display an increased survival in response to DNA-damaging agents like doxorubicin. Maybe the FGFR4 Arg388/385 enables the cell to survive DNA-damaging and occurring genomic instability, which is a typical event in cellular transformation. If FGFR4 Arg385 expressing cells could easily deal genomic instability, they could consequently easier transform. Further, the FGFR4 Arg385/388 eventually supports DNA-repair mechanisms and thereby permits a faster and more effective DNA-repair. Hence a lower percentage of cells would enter apoptosis in response to DNA-damage.

However, our data suggest the FGFR4 Arg385 allele is a potent enhancer of breast tumor development and progression in vivo. Hence, further studies on our generated knock-in mouse should investigate a possible impact of the FGFR4 Arg385 allele also in other cancers, for example liver cancer. Here, several recent publications implicate the FGFR4 in liver functions and homeostasis (34). Further the development of a therapeutic antibody blocking the FGFR4 could possibly be used additionally with classical cancer therapies like chemotherapeutic drugs. Furthermore, the FGFR4 could not only be additionally targeted, but the genomic disposition of this receptor would also be conceivable to be included in the decision of cancer therapy with the according patient. This notion is also strongly supported by Thussbass and colleagues, who could show that a different time of relapse of treated mammary tumors after different drug-treatments is associated with the different FGFR4 alleles (16).

Recapitulatory, the implication of the FGFR4 and especially the tumor progressive impact of the Arg385 allele can not be negated. Our report suggests a role of the FGFR4 Arg385 allele as a marker for poor clinical outcome in breast cancer progression and metastasis. Furthermore, these observations highlight the impact of germline alteration and especially single nucleotide substitutions in receptor tyrosine kinase genes for the clinical progression of cancer and thereby validate FGFR4 as possible target for the development of prototypical drugs for individualized cancer therapies.

### REFERENCES

1. Jeffers, M., LaRochelle, W. J., and Lichenstein, H. S. 2002. Fibroblast growth factors in cancer: therapeutic possibilities. Expert Opin Ther Targets 6:469-482.
2. Burke, D., Wilkes, D., Blundell, T. L., and Malcolm, S. 1998. Fibroblast growth factor receptors: lessons from the genes. Trends Biochem Sci 23:59-62.
3. Eswarakumar, V. P., Lax, L, and Schlessinger, J. 2005. Cellular signaling by fibroblast growth factor receptors. Cytokine Growth Factor Rev 16:139-149.
4. Gowardhan, B., Douglas, D. A., Mathers, M. E., McKie, A. B., McCracken, S. R., Robson, C. N., and Leung, H. Y. 2005. Evaluation of the fibroblast growth factor system as a potential target for therapy in human prostate cancer. Br J Cancer 92:320-327.
5. Morrison, R. S., Yamaguchi, F., Bruner, J. M., Tang, M., McKeehan, W., and Berger, M. S. 1994. Fibroblast growth factor receptor gene expression and immunoreactivity are elevated in human glioblastoma multiforme. Cancer Res 54:2794-2799.
6. Cappellen, D., De Oliveira, C., Ricol, D., de Medina, S., Bourdin, J., Sastre-Garau, X., Chopin, D., Thiery, J. P., and Radvanyi, F. 1999. Frequent activating mutations of FGFR3 in human bladder and cervix carcinomas. Nat Genet 23:18-20.
7. Jang, J. H., Shin, K. H., and Park, J. G. 2001. Mutations in fibroblast growth factor receptor 2 and fibroblast growth factor receptor 3 genes associated with human gastric and colorectal cancers. Cancer Res 61:3541-3543.
8. Ameyaw, M. M., Tayeb, M., Thornton, N., Folayan, G., Tariq, M., Mobarek, A., Evans, D. A., Ofori-Adjei, D., and McLead, H. L. 2002. Ethnic variation in the HER-2 codon 655 genetic polymorphism previously associated with breast cancer. J Hum Genet 47:172-175.
9. Przybylowska, K., Smolarczyk, K., Blasiak, J., Kulig, A., Romanowicz-Makowska, H., Dziki, A., Ulanska, J., and Pander, B. 2001. A CIT polymorphism in the urokinase-type plasminogen activator gene in colorectal cancer. J Exp Clin Cancer Res 20:569-572.
10. Morimoto, Y., Ozaki, T., Ouchida, M., Umehara, N., Ohata, N., Yoshida, A., Shimizu, K., and Inoue, H. 2003. Single nucleotide polymorphism in fibroblast growth factor receptor 4 at codon 388 is associated with prognosis in high-grade soft tissue sarcoma. Cancer 98:2245-2250.
11. Bange, J., Prechtl, D., Cheburkin, Y., Specht, K., Harbeck, N., Schmitt, M., Knyazeva, T., Muller, S., Gartner, S., Sures, I., et al. 2002. Cancer progression and tumor cell motility are associated with the FGFR4 Arg(388) allele. Cancer Res 62:840-847.
12. Streit, S., Bange, J., Fichtner, A., Ihrler, S., Issing, W., and Ullrich, A. 2004. Involvement of the FGFR4 Arg388 allele in head and neck squamous cell carcinoma. Int J Cancer 111:213-217.
13. Streit, S., Mestel, D. S., Schmidt, M., Ullrich, A., and Berking, C. 2006. FGFR4 Arg388 allele correlates with tumor thickness and FGFR4 protein expression with survival of melanoma patients. Br J Cancer 94:1879-1886.
14. Wang, J., Stockton, D. W., and Ittmann, M. 2004. The fibroblast growth factor receptor-4 Arg388 allele is associated with prostate cancer initiation and progression. Clin Cancer Res 10:6169-6178.
15. Spinola, M., Leoni, V., Pignatiello, C., Conti, B., Ravagnani, F., Pastorino, U., and Dragani, T. A. 2005. Functional FGFR4 Gly388Arg polymorphism predicts prognosis in lung adenocarcinoma patients. J Clin Oncol 23:7307-7311.
16. Thussbas, C., Nahrig, J., Streit, S., Bange, J., Kriner, M., Kates, R., Ulm, K., Kiechle, M., Hoefler, H., Ullrich, A., et al. 2006. FGFR4 Arg388 allele is associated with resistance to adjuvant therapy in primary breast cancer. J Clin Oncol 24:3747-3755.
17. Jezequel, P., Campion, L., Joalland, M. P., Millour, M., Dravet, F., Classe, J. M., Delecroix, V., Deporte, R., Fumoleau, P., and Ricolleau, G. 2004. G388R mutation of the FGFR4 gene is not relevant to breast cancer prognosis. Br J Cancer 90:189-193.
18. Spinola, M., Leoni, V. P., Tanuma, J., Pettinicchio, A., Frattini, M., Signoroni, S., Agresti, R., Giovanazzi, R., Pilotti, S., Bertario, L., et al. 2005. FGFR4 Gly388Arg polymorphism and prognosis of breast and colorectal cancer. Oncol Rep 14:415-419.
19. Pittius, C. W., Hennighausen, L., Lee, E., Westphal, H., Nicols, E., Vitale, J., and Gordon, K. 1988. A milk protein gene promoter directs the expression of human tissue plasminogen activator cDNA to the mammary gland in transgenic mice. Proc Natl Acad Sci USA 85:5874-5878.
20. Sandgren, E. P., Schroeder, J. A., Qui, T. H., Palmiter, R. D., Brinster, R. L., and Lee, D. C. 1995. Inhibition of mammary gland involution is associated with transforming growth factor alpha but not c-myc-induced tumorigenesis in transgenic mice. Cancer Res 55:3915-3927.
21. Messing, J. 1983. In vitro DNA synthesis as a tool to analyze and alter genes. Basic Life Sci 25:9-15.
22. Kunkel, T. A. 1985. The mutational specificity of DNA polymerases-alpha and -gamma during in vitro DNA synthesis. J Biol Chem 260:12866-12874.
23. Hooper, M., Hardy, K., Handyside, A., Hunter, S., and Monk, M. 1987. HPRT-deficient (Lesch-Nyhan) mouse embryos derived from germline colonization by cultured cells. Nature 326:292-295.
24. Southern, E. M. 1974. An improved method for transferring nucleotides from electrophoresis strips to thin layers of ion-exchange cellulose. Anal Biochem 62:317-318.
25. Todaro, G. J., and Green, H. 1963. Quantitative studies of the growth of mouse embryo cells in culture and their development into established lines. J Cell Biol 17:299-313.
26. Pear, W. S., Nolan, G. P., Scott, M. L., and Baltimore, D. 1993. Production of high-titer helper-free retroviruses by transient transfection. Proc Natl Acad Sci USA 90:8392-8396.
27. Nicoletti, L, Migliorati, G., Pagliacci, M. C., Grignani, F., and Riccardi, C. 1991. A rapid and simple method for measuring thymocyte apoptosis by propidium iodide staining and flow cytometry. J Immunol Methods 139:271-279.
28. Wakabayashi, Y., Mao, J. H., Brown, K., Girardi, M., and Balmain, A. 2007. Promotion of Hras-induced squamous carcinomas by a polymorphic variant of the Patched gene in FVB mice. Nature 445:761-765.
29. Partanen, J., Makela, T. P., Eerola, E., Korhonen, J., Hirvonen, H., Claesson-Welsh, L., and Alitalo, K. 1991. FGFR-4, a novel acidic fibroblast growth factor receptor with a distinct expression pattern. Embo J 10:1347-1354.
30. Ezzat, S., Zheng, L., Zhu, X. F., Wu, G. E., and Asa, S. L. 2002. Targeted expression of a human pituitary tumor-derived isoform of FGF receptor-4 recapitulates pituitary tumorigenesis. J Clin Invest 109:69-78.
31. Jaakkola, S., Salmikangas, P., Nylund, S., Partanen, J., Armstrong, E., Pyrhonen, S., Lehtovirta, P., and Nevanlinna, H. 1993. Amplification of fgfr4 gene in human breast and gynecological cancers. Int J Cancer 54:378-382.
32. Guy, C. T., Cardiff, R. D., and Muller, W. J. 1992. Induction of mammary tumors by expression of polyomavirus middle T oncogene: a transgenic mouse model for metastatic disease. Mol Cell Biol 12:954-961.
33. Stadler, C. R., Knyazev, P., Bange, J., and Ullrich, A. 2006. FGFR4 GLY388 isotype suppresses motility of MDA-MB-231 breast cancer cells by EDG-2 gene repression. Cell Signal 18:783-794.
34. Huang X, Yang C, Luo Y, Jin C, Wang F, McKeehan W L. FGFR4 prevents hyperlipidemia and insulin resistance but underlies high-fat diet induced fatty liver. Diabetes, October 2007.
35. Bange, J., Prechtl, D., Cheburkin, Y., et al. Cancer progression and tumor cell motility are associated with the FGFR4 Arg(388) allele. Cancer Res, 2002; 62(3): 840-847.
36. Wang, J., Cai, Y., Penland, R., Chauhan, S., Miesfeld, R. L., and Ittmann, M. (2006). Increased expression of the metastasis-associated gene Ehm2 in prostate cancer. Prostate 66, 1641-1652.
37. Higgins and Sharp (1988), Gene 73; 237-44.
38. Carpet F (1988), Nucleic Acid Research 16: 10881-90.
39. Tatusova et al. (1999), FMES Microbiol. Lett. 174: 247-50.
40. Guy et al. (1992), Mol. Cell. Biol. 12: 954-61.
41. Yu et al. (2000), J. Biol. Chem. 275: 15482-9.
42. Andersen, J. S., Lam, Y. W., Leung, A. K., Ong, S. E., Lyon, C. E., Lamond, A. I., and Mann, M. (2005). Nucleolar proteome dynamics. Nature 433, 77-83.
43. Bashour, A. M., and Bloom, G. S. (1998). 58K, a microtubule-binding Golgi protein, is a formiminotransferase cyclodeaminase. J Biol Chem 273, 19612-19617.
44. Camarero, N., Mascaro, C., Mayordomo, C., Vilardell, F., Haro, D., and Marrero, P. F. (2006). Ketogenic HMGCS2 Is a c-Myc target gene expressed in differentiated cells of human colonic epithelium and down-regulated in colon cancer. Mol Cancer Res 4, 645-653.
45. Carles-Kinch, K., Kilpatrick, K. E., Stewart, J. C., and Kinch, M. S. (2002). Antibody targeting of the EphA2 tyrosine kinase inhibits malignant cell behavior. Cancer Res 62, 2840-2847.
46. Ferdinandusse, S., Kostopoulos, P., Denis, S., Rusch, H., Overmars, H., Dillmann, U., Reith, W., Haas, D., Wanders, R. J., Duran, M., and Marziniak, M. (2006). Mutations in the gene encoding peroxisomal sterol carrier protein X (SCPx) cause leukencephalopathy with dystonia and motor neuropathy. Am J Hum Genet 78, 1046-1052.
47. Fuchs, M., Hafer, A., Munch, C., Kannenberg, F., Teichmann, S., Scheibner, J., Stange, E. F., and Seedorf, U. (2001). Disruption of the sterol carrier protein 2 gene in mice impairs biliary lipid and hepatic cholesterol metabolism. J Biol Chem 276, 48058-48065.
48. Herbst, R. S., Fukuoka, M., and Baselga, J. (2004). Gefitinib—a novel targeted approach to treating cancer. Nat Rev Cancer 4, 956-965.
49. Huang, X., Yang, C., Jin, C., Luo, Y., Wang, F., and McKeehan, W. L. (2008). Resident hepatocyte fibroblast growth factor receptor 4 limits hepatocarcinogenesis. Mol Carcinog.
50. Huang, X., Yang, C., Luo, Y., Jin, C., Wang, F., and McKeehan, W. L. (2007). FGFR4 prevents hyperlipidemia and insulin resistance but underlies high-fat diet induced fatty liver. Diabetes 56, 2501-2510.
51. Kruger, M., Moser, M., Ussar, S., Thievessen, I., Luber, C. A., Forner, F., Schmidt, S., Zanivan, S., Fassler, R., and Mann, M. (2008). SILAC mouse for quantitative proteomics uncovers kindlin-3 as an essential factor for red blood cell function. Cell 134, 353-364.
52. Kurosu, H., and Kuro, O. M. (2009). The Klotho gene family as a regulator of endocrine fibroblast growth factors. Mol Cell Endocrinol 299, 72-78.
53. Lapierre, P., Hajoui, O., Homberg, J. C., and Alvarez, F. (1999). Formiminotransferase cyclodeaminase is an organ-specific autoantigen recognized by sera of patients with autoimmune hepatitis. Gastroenterology 116, 643-649.
54. Llovet, J. M., and Bruix, J. (2008). Molecular targeted therapies in hepatocellular carcinoma. Hepatology 48, 1312-1327.
55. Machide, M., Hashigasako, A., Matsumoto, K., and Nakamura, T. (2006). Contact inhibition of hepatocyte growth regulated by functional association of the c-Met/hepatocyte growth factor receptor and LAR protein-tyrosine phosphatase. J Biol Chem 281, 8765-8772.
56. Mander, A., Hodgkinson, C. P., and Sale, G. J. (2005). Knock-down of LAR protein tyrosine phosphatase induces insulin resistance. FEBS Lett 579, 3024-3028.
57. McArdle, L., Rafferty, M. M., Satyamoorthy, K., Maelandsmo, G. M., Dervan, P. A., Herlyn, M., and Easty, D. J. (2005). Microarray analysis of phosphatase gene expression in human melanoma. Br J Dermatol 152, 925-930.
58. Nadal, A., Marrero, P. F., and Haro, D. (2002). Down-regulation of the mitochondrial 3-hydroxy-3-methylglutaryl-CoA synthase gene by insulin: the role of the forkhead transcription factor FKHRL1. Biochem J 366, 289-297.
59. Noblitt, L. W., Bangari, D. S., Shukla, S., Knapp, D. W., Mohammed, S., Kinch, M. S., and Mittal, S. K. (2004). Decreased tumorigenic potential of EphA2-overexpressing breast cancer cells following treatment with adenoviral vectors that express EphrinA1. Cancer Gene Ther 11, 757-766.
60. Ong, S. E., and Mann, M. (2006). A practical recipe for stable isotope labeling by amino acids in cell culture (SILAC). Nat Protoc 1, 2650-2660.
61. Recalcati, S., Tacchini, L., Alberghini, A., Conte, D., and Cairo, G. (2003). Oxidative stress-mediated down-regulation of rat hydroxyacid oxidase 1, a liver-specific peroxisomal enzyme. Hepatology 38, 1159-1166.
62. Ruhe, J. E., Streit, S., Hart, S., and Ullrich, A. (2006). EGFR signaling leads to downregulation of PTP-LAR via TACE-mediated proteolytic processing. Cell Signal 18, 1515-1527.
63. Shevchenko, A., Wilm, M., Vorm, O., and Mann, M. (1996). Mass spectrometric sequencing of proteins silver-stained polyacrylamide gels. Anal Chem 68, 850-858.
64. Stadler, C. R., Knyazev, P., Bange, J., and Ullrich, A. (2006). FGFR4 GLY388 isotype suppresses motility of MDA-MB-231 breast cancer cells by EDG-2 gene repression. Cell Signal 18, 783-794.
65. Wang, J., Cai, Y., Penland, R., Chauhan, S., Miesfeld, R. L., and lttmann, M. (2006). Increased expression of the metastasis-associated gene Ehm2 in prostate cancer. Prostate 66, 1641-1652.
66. Wang, J., Yu, W., Cai, Y., Ren, C., and Ittmann, M. M. (2008). Altered fibroblast growth factor receptor 4 stability promotes prostate cancer progression. Neoplasia 10, 847-856.
67. Wang, K., Wang, K., Li, W., Huang, T., Li, R., Wang, D., Shen, B., and Chen, X. (2009). Characterizing Breast Cancer Xenograft Epidermal Growth Factor Receptor Expression by Using Near-Infrared Optical Imaging. Acta Radiol, 1-9.
68. Wang, X., Weng, L. P., and Yu, Q. (2000). Specific inhibition of FGF-induced MAPK activation by the receptor-like protein tyrosine phosphatase LAR. Oncogene 19, 2346-2353.
69. Weng, L. P., Yuan, J., and Yu, Q. (1998). Overexpression of the transmembrane tyrosine phosphatase LAR activates the caspase pathway and induces apoptosis. Curr Biol 8, 247-256.
70. Wu, X., Ge, H., Gupte, J., Weiszmann, J., Shimamoto, G., Stevens, J., Hawkins, N., Lemon, B., Shen, W., Xu, J., et al. (2007). Co-receptor requirements for fibroblast growth factor-19 signaling. J Biol Chem 282, 29069-29072.
71. Yu, C., Wang, F., Kan, M., Jin, C., Jones, R. B., Weinstein, M., Deng, C. X., and McKeehan, W. L. (2000). Elevated cholesterol metabolism and bile acid synthesis in mice lacking membrane tyrosine kinase receptor FGFR4. J Biol Chem 275, 15482-15489.
72. Zheng, X., Jiang, F., Katakowski, M., Zhang, Z. G., Lu, Q. E., and Chopp, M. (2009). ADAM17 promotes breast cancer cell malignant phenotype through EGFR-PI3K-AKT activation. Cancer Biol Ther 8.

1. A rodent animal comprising an endogenous gene encoding a modified FGFR4 protein, wherein the modification is an amino acid substitution in the wild-type FGFR4 of said rodent at the amino acid position corresponding to amino acid position 385 of SEQ ID NO: 1.

2. The animal of claim 1 wherein the rodent is a mouse, hamster or rat, preferably a mouse.

3. The animal of claim 1 wherein the rodent is a mouse and wherein said amino acid position is amino acid position 385 of SEQ ID NO: 1.

4. The animal of claim 1 wherein the rodent is a rat and wherein said amino acid position is amino acid position 386 of SEQ ID NO: 4.

5. The animal according to claim 1 wherein in said rodent the amino acid position corresponding to amino acid position 385 of SEQ ID NO: 1 is glycine.

6. The animal according to claim 1 wherein in said rodent the amino acid substitution is with an amino acid different from glycine.

7. The animal according to claim 1, wherein said amino acid substitution is with an amino acid with a charged side chain, preferably a lysine, arginine or histidine, and more preferably an arginine.

8. The animal according to claim 1, wherein the modified FGFR4 has the amino acid sequence of SEQ ID NO: 5 or SEQ ID NO: 6.

9. The animal according to claim 1, wherein at least some of its cells or all cells of said non-human animal comprise said endogenous modified FGFR4 encoding gene.

10. The animal according to claim 1, wherein the at least some cells or all cells are heterozygous or homozygous with respect to said modified FGFR4.

11. The animal according to claim 1, additionally displaying uncontrolled cell growth, preferably cancer and/or metastasis formation.

12. The animal according to claim 1, further being irradiated, treated with cancer-inducing agent and/or comprising a transgene, wherein said transgene comprises an oncogene.

13. The animal according to claim 12 wherein
a the oncogene is TGF-α (SEQ ID NO: 74), TGF-β, EGFR (SEQ ID NO: 76), v-src, c-kit, HER2, erb-B2, p53, myc, or/and ras; and/or
b the cancer-inducing agent is dimethylhydrazine (DMH), azoxymethane (AOM), N-methyl-N-nitro-N-nitrosoguanidine (MNNG), N-methyl-N-nitrosourea (MNU), ethyl-nitroso-urea (ENU) or 12-0-tetradecanoylphorbol-13-acetate (TPA).

14. The animal according to claim 12, wherein said transgene is expressed in mammary cells and/or hepatocytes.

15. The animal of claim 1 wherein the modification of said FGFR4 results in a phenotype associated with an alteration in tumor progression and/or formation.

16. The animal of claim 14 wherein the alteration in tumor progression is characterized by an increased rate of tumor growth and/or metastasis formation compared to a wild-type animal.

17. Primary cells or a cell line derived from the animal of claim 1, wherein said primary cells are preferably mouse embryonic feeder cells (MEFs).

18. The primary cells or cell line d claim 17, wherein said cells or cell lines are homozygous or heterozygous with respect to the modified FGFR4.

19. The primary cells or cell line of claim 17, wherein said cells or cell lines line comprise a nucleic acid encoding for EGFR (SEQ ID NO: 76) or EGFR protein (SEQ ID NO: 77).

20. Use of the animal, primary cells, or cell lines according to claim 1 as a model for:
(a) studying the molecular mechanisms of, or physiological processes associated with uncontrolled cell growth, such as cancer and/or metastasis formation, preferably in breast cancer, lung cancer, colorectal cancer, hepatocellular cancer, prostate cancer, melanoma, and/or pancreatic cancer;
(b) identification and/or testing of an agent useful in the prevention, amelioration or treatment of uncontrolled cell growth, such as cancer and/or metastasis formation, preferably in breast cancer, lung cancer, colorectal cancer, hepatocellular cancer, prostate cancer, melanoma, and/or pancreatic cancer;
(c) identification of a protein and/or nucleic diagnostic marker for uncontrolled cell growth, such as cancer and/or metastasis formation, preferably in breast cancer, lung cancer, colorectal cancer, hepatocellular cancer, prostate cancer, melanoma, and/or pancreatic cancer; and/or
(d) studying the molecular mechanisms of, or physiological processes or medical conditions associated with undesirable activity, expression, or production of said modified FGFR4.

21. A modified FGFR4 polypeptide according to SEQ ID NO: 5 or SEQ ID NO: 6.

22. A nucleic acid molecule encoding the polypeptide of claim 21.

23. An expression vector comprising the nucleic acid of claim 22.

24. A host cell comprising the polypeptide of claim 21.

25. A rodent animal comprising an endogenous gene encoding a modified FGFR4 protein, wherein the modification is at least one amino acid substitution compared to the wild-type FGFR4 protein, preferably an FGFR4 protein according to SEQ ID NO: 1, which modification, if present in at least some or all or essentially all cells of said animal in a heterozygous or homozygous manner, results in a phenotype associated with an increased rate of tumor growth and/or metastasis formation compared to a wild-type animal.

26. A rodent animal according to claim 25 which additionally expresses in the genome of at least some of its cells a transgene encoding a TGF-α protein.

27. The animal of claim 26 wherein said transgene is expressed in mammary cells, preferably by expression under the control of the WAP-promotor.

28. The animal according to claim 25, wherein said tumor is a mammary tumor.

29. Method of identifying an agent inhibiting the interaction between a modified FGFR4 protein and EGFR protein comprising:
(a) culturing cell(s) overexpressing a modified FGFR4 protein;
(b) adding the agent to be tested to the culture medium; and
(c) determining a decrease in the proliferation rate, an increase in apoptosis and/or a decrease in cell migration of the cell(s) cultured in the presence of the agent compared to (a) cell(s) overexpressing a wild-type FGFR4 protein cultured in the presence of the same agent.

30. The method according to claim 29 wherein the modified FGFR4 protein is a protein of SEQ ID NO: 5 and the wild-type FGFR4 protein is a protein of SEQ ID NO: 1.

31. The method according to claim 29 wherein the cell(s) is/are (a) MDA-MB-231 cell(s).

32. The method according to claim 29 wherein the proliferation rate is determined by an MTT proliferation assay, the apoptosis is determined by FACS analysis, and/or the migration is determined with a Boyden Chamber Assay.

33. An inhibitor of FGFR4 for the treatment of an EGFR-associated disorder.

34. An inhibitor of FGFR4 according to claim 33 wherein the EGFR-associated disorder is an EGF and/or TGF-alpha mediated disorder.

35. An inhibitor of FGFR4 according to claim 33 wherein the EGFR-associated disorder is cancer, preferably breast cancer or heptocellular cancer.

36. An inhibitor of FGFR4 according to claim 33 wherein the inhibitor is selected from the group consisting of an antibody directed against FGFR4, an aptamer directed against FGFR4, an antisense oligonucleotide directed against FGFR4, and a RNAi molecule directed against FGFR4.

37. An inhibitor of FGFR4 according to claim 33 the FGFR4 protein is a human FGFR4 protein, particularly of SEQ ID NO:2 or SEQ ID NO:3.

38. An inhibitor of FGFR4 according to claim 33 wherein the FGFR4 protein is a modified human FGFR4 protein wherein the modification is an amino acid substitution/of the amino acid glycine at the amino acid position 388 of SEQ ID NO:2 or SEQ ID NO:3, preferably a substitution with arginine.

39. A method of diagnosing severe cancer progression by
(a) determining the expression of EGFR gene or protein; and/or
(b) determining the interaction between FGFR4 protein and EGFR protein; and/or
(c) determining the stimulation of EGFR protein by TGF-alpha and/or EGF; and/or
(d) determining whether FGFR4 is the wild-type protein or gene, particularly of SEQ ID NO:2 or SEQ ID NO:3 or a modified human FGFR4 protein wherein the modification is an amino acid substitution of the amino acid glycine at the amino acid position 388 of SEQ ID NO:2 or SEQ ID NO:3, preferably a substitution with arginine wherein an upregulation of the expression of EGFR gene or protein; an upregulation of the stimulation of EGFR protein by TGF-alpha and/or EGF; and/or the presence of said modified human FGFR4 protein is indicative for severe cancer progression.

40. A host cell comprising the nucleic acid of claim 22.

41. A host cell comprising the expression vector of claim 23.
